(19) United States
(12) Patent Application Publication
(10) Pub. No.: US 2024/0002462 A1
(43) Pub. Date: Jan. 4, 2024

(54) TREATMENT OF NEUROPATHY WITH IGF-1-ENCODING DNA CONSTRUCTS AND HGF-ENCODING DNA CONSTRUCTS

(71) Applicant: Helixmith Co., Ltd, Seoul (KR)

(72) Inventors: Junghun LEE, Seoul (KR); Nayeon LEE, Seoul (KR); Kyeong Ryang KO, Seoul (KR)

(21) Appl. No.: 18/054,606

(22) Filed: Nov. 11, 2022

Related U.S. Application Data
(63) Continuation of application No. 16/513,560, filed on Jul. 16, 2019, now abandoned.
(60) Provisional application No. 62/699,667, filed on Jul. 17, 2018.

(51) Int. Cl. C07K 14/65; C12N 15/63; C07K 14/475

(52) U.S. Cl. CPC C07K 14/65; C12N 15/63; C07K 14/4753; A61K 38/00

(57) ABSTRACT

The present invention relates to a method of treating neuropathy by administering DNA constructs, encoding a human IGF-1 isoform and a human HGF isoform. Further provided herein are various DNA constructs and pharmaceutical compositions including the DNA constructs that can be used for the combination therapy. The present invention provides a safe and effective way of treating neuropathic patients.

### 1. CROSS-REFERENCE TO RELATED APPLICATIONS

This application is a continuation of U.S. application Ser. No. 16/513,560, filed Jul. 16, 2019, which claims priority to U.S. Provisional Application No. 62/699,667, filed Jul. 17, 2018, each of which is hereby incorporated by reference in its entirety.

### 2. SEQUENCE LISTING

The instant application contains a Sequence Listing which has been submitted via Patent Center and is hereby incorporated by reference in its entirety. Said XML copy, created on Aug. 28, 2023, is named 54125US_CRF_sequencelisting.xml and is 128,653 bytes in size.

### 3. BACKGROUND

Neuropathy is a chronic pathologic condition resulting from nerve damage. Neuropathy is a common consequence of diabetes, with neuropathy in a diabetic patient specifically referred to as diabetic neuropathy. Neuropathy can also be caused by nerve damage caused by infections (e.g., herpes, with the associated neuropathy arising after infection known as post-herpetic neuralgia; HIV/AIDS; Lyme disease: leprosy; syphilis; and shingles); autoimmune diseases (e.g., rheumatoid arthritis, systemic lupus, and Guillain-Barre syndrome); genetic or inherited disorders (e.g., Friedreich's ataxia and Charcot-Marie-Tooth disease); amyloidosis; uremia; exposure to toxins, poisons or drugs; trauma; or injury. In some cases, the cause is not known, in which case the neuropathy is referred to as idiopathic neuropathy.

Regardless of the cause, neuropathy is associated with characteristic symptoms that depend, in part, on the anatomic site of nerve damage (e.g., peripheral neuropathy, cranial neuropathy, autonomic neuropathy, focal neuropathy), such as pain (neuropathic pain), other sensory defects (e.g., anesthesias, including partial or complete loss of feeling; and paresthesias, including numbness, tingling, etc.), motor defects (e.g., weakness, loss of reflexes, loss of muscle mass, cramping, loss of dexterity, etc.), and autonomic dysfunction (e.g., nausea, vomiting, impotence, dizziness, constipation, diarrhea, etc.).

Neuropathy is routinely treated with measures that manage associated symptoms, and when the etiology is known, by treating the underlying cause of neuropathy. For example, pain medications, or medical treatments for diabetes, autoimmune diseases, infections, or vitamin deficiencies have been used. However, these methods do not treat the nerve damage itself.

There is, therefore, a need for an effective treatment method that can prevent and repair nerve damage associated with neuropathy.

Various growth factors have been suggested as possible agents for treating neuropathy, and Kessler and colleagues recently reported a successful double-blind, placebo-controlled, phase 2 human clinical trial of hepatocyte growth factor (HGF) nonviral gene therapy in diabetic peripheral neuropathy. Kessler et al., Annals Clin. Transl. Neurology 2(5):465-478 (2015). See also U.S. Pat. No. 9,963,493, incorporated herein by reference in its entirety.

Given the wide range of etiologies that cause neuropathy and the wide range of neuropathy clinical presentations, despite the clinical success in treating diabetic peripheral neuropathy with HGF-expressing DNA constructs, there remains a need for additional treatments, including treatments using HGF with other therapeutic agents.

### 4. SUMMARY

The present invention is based on a novel finding that administration of an IGF-1-encoding DNA construct capable of expressing a human IGF-1 isoform and a HGF-encoding DNA construct capable of expressing a human HGF isoform in combination is effective in treating a symptom associated with neuropathy. The treatment effects of the two DNA constructs in combination were demonstrated to be greater than the treatment effects of an HGF-encoding DNA construct by itself (e.g., VM202 or pCK-HGF₇₂₈). The present invention further provides various DNA constructs encoding an IGF-1 isoform or an HGF isoform that can be used for the combination therapy. Further provided herein are methods of administering the DNA constructs, demonstrated to be effective in treating symptoms associated with neuropathy in vivo.

Thus, the present invention provides a novel combination therapy using IGF-1 and HGF isoforms for treating neuropathy.

Specifically, in one aspect, the present invention provides a method of treating neuropathy, comprising the steps of: (1) administering to a subject having neuropathy a therapeutically effective amount of a first IGF-1-encoding DNA construct capable of expressing a human IGF-1 isoform; and (2) administering to the subject a therapeutically effective amount of first HGF-encoding DNA construct capable of expressing a human HGF isoform.

In some embodiments, the first IGF-1-encoding DNA construct is capable of expressing Class I IGF-1Ea protein comprising a polypeptide of SEQ ID NO: 14 or Class I IGF-1Ec protein comprising a polypeptide of SEQ ID NO: 16. In some embodiments, the first IGF-1-encoding DNA construct is not capable of expressing both Class II IGF-1 Ea protein comprising a polypeptide of SEQ ID NO: 18 and Class I IGF-1Eb protein comprising a polypeptide of SEQ ID NO: 20.

In some embodiments, the first IGF-1-encoding DNA construct comprises a polynucleotide of SEQ ID NO: 15. In some embodiments, the method further comprises the step of administering to the subject a second IGF-1-encoding DNA construct, wherein the second IGF-1-encoding DNA construct comprises a polynucleotide of SEQ ID NO: 17.

In some embodiments, the first IGF-1-encoding DNA construct comprises a polynucleotide of SEQ ID NO: 17. In some embodiments, the method further comprises the step of administering to the subject a second IGF-1-encoding DNA construct, wherein the second IGF-1-encoding DNA construct comprises a polynucleotide of SEQ ID NO: 15.

In some embodiments, the step of administering the first IGF-1-encoding DNA construct and the step of administering the second IGF-1-encoding DNA construct are performed concurrently. In some embodiments, the step of administering the first IGF-1-encoding DNA construct and the step of administering the second IGF-1-encoding DNA construct are performed sequentially.

In some embodiments, the first IGF-1-encoding DNA construct encodes more than one human IGF-1 isoforms. In some embodiments, the more than one human IGF-1 isoforms comprise a polypeptide of SEQ ID NO: 14 and a polypeptide of SEQ ID NO: 16.

In some embodiments, the first IGF-1-encoding DNA construct comprises: a first IGF polynucleotide of SEQ ID NO: 1 (exons 1, 3, 4) or a degenerate thereof, a second IGF polynucleotide of SEQ ID NO: 2 (intron 4) or a fragment thereof, a third IGF polynucleotide of SEQ ID NO: 3 (exons 5 and 6-1) or a degenerate thereof, a fourth IGF polynucleotide of SEQ ID NO: 4 (intron 5) or a fragment thereof, and a fifth IGF polynucleotide of SEQ ID NO: 5 (exon 6-2) or a degenerate thereof, wherein the first polynucleotide, the second polynucleotide, the third polynucleotide, the fourth polynucleotide and the fifth polynucleotide are linked in sequential 5′ to 3′ order.

In some embodiments, the second IGF polynucleotide is a polynucleotide of SEQ ID NO: 6. In some embodiments, the second IGF polynucleotide is a polynucleotide of SEQ ID NO: 7. In some embodiments, the fourth IGF polynucleotide is a polynucleotide of SEQ ID NO: 8.

In some embodiments, the first IGF-1-encoding DNA construct comprises a plasmid vector. In some embodiments, the plasmid vector is pCK. In some embodiments, the plasmid vector is pTx.

In some embodiments, the first IGF-1-encoding DNA construct comprises a polynucleotide of SEQ ID NO: 10. In some embodiments, the first IGF-1-encoding DNA construct comprises a polynucleotide of SEQ ID NO: 9.

In some embodiments, the first IGF-1-encoding DNA construct and the first HGF-encoding DNA construct are administered in an amount sufficient to reduce pain in the subject. In some embodiments, the subject has diabetic neuropathy.

In some embodiments, the first IGF-1-encoding DNA construct and the first HGF-encoding DNA construct are administered by a plurality of intramuscular injections.

In some embodiments, the human HGF isoform is flHGF of SEQ ID NO: 11 or dHGF of SEQ ID NO: 12.

In some embodiments, the first HGF-encoding DNA construct encodes more than one human HGF isoforms. In some embodiments, the first HGF-encoding DNA construct encodes two human HGF isoforms, wherein the two human HGF isoforms are flHGF of SEQ ID NO: 11 and dHGF of SEQ ID NO: 12.

In some embodiments, the first HGF-encoding DNA construct comprises a plasmid vector, optionally wherein the plasmid vector is a pCK vector or a pTx vector.

In some embodiments, the first HGF-encoding DNA construct comprises: a first HGF polynucleotide of exons 1-4 of SEQ ID NO: 22 or a degenerate thereof, a second HGF polynucleotide of intron 4 of SEQ ID NO: 25 or a functional fragment thereof, and a third HGF polynucleotide of exons 5-18 of SEQ ID NO: 23 or a degenerate thereof, wherein the second HGF polynucleotide is located between the first HGF polynucleotide and the third HGF polynucleotide, and the first HGF-encoding DNA construct encodes two human HGF isoforms.

In some embodiments, the first HGF-encoding DNA construct comprises a polynucleotide of SEQ ID NO: 13.

In some embodiments, the first IGF-1-encoding DNA construct and the first HGF-encoding DNA construct are co-administered. In some embodiments, the first IGF-1-encoding DNA construct and the first HGF-encoding DNA construct are co-administered by an intramuscular injection.

In some embodiments, the step of administering the first IGF-1-encoding DNA construct and the step of administering the first HGF-encoding DNA construct are performed separately.

In some embodiments, the step of administering the first IGF-1-encoding DNA construct and the step of administering the first HGF-encoding DNA construct are performed at least three weeks apart.

In some embodiments, the method further comprises the step of administering to the subject a second HGF-encoding DNA construct capable of expressing a human HGF isoform selected from flHGF of SEQ ID NO: 11 and dHGF of SEQ ID NO: 12.

In some embodiments, the method comprises the steps of: administering to a subject having neuropathy an HGF-encoding DNA construct comprising a polynucleotide of SEQ ID NO: 13; and administering to the subject an IGF-1-encoding DNA construct comprising a polynucleotide of SEQ ID NO: 10 or a polynucleotide of SEQ ID NO: 9, wherein the step of administering the HGF-encoding DNA construct and the step of administering the IGF-1-encoding DNA construct are performed at least three weeks apart.

In some embodiments, the method comprises the steps of: administering to a subject having neuropathy an HGF-encoding DNA construct comprising a polynucleotide of SEQ ID NO: 33; and administering to the subject an IGF-1-encoding DNA construct comprising a polynucleotide of SEQ ID NO: 10 or a polynucleotide of SEQ ID NO: 9, wherein the step of administering the HGF-encoding DNA construct and the step of administering the IGF-1-encoding DNA construct are performed at least three weeks apart.

In some embodiments, the steps of: administering to a subject having neuropathy an HGF-encoding DNA construct comprising a polynucleotide of SEQ ID NO: 13; and administering to the subject a first IGF-1-encoding DNA construct comprising a polynucleotide of SEQ ID NO: 15 and a second IGF-1-encoding DNA construct comprising a polynucleotide of SEQ ID NO: 17, wherein the step of administering the HGF-encoding DNA construct and the step of administering the first IGF-1-encoding DNA construct and the second IGF-1-encoding DNA construct are performed at least three weeks apart.

In another aspect, the present invention provides a pharmaceutical composition comprising: an IGF-1-encoding DNA construct capable of expressing at least one human IGF-1 isoform; an HGF-encoding DNA construct capable of expressing at least one human HGF isoform, and a pharmaceutically acceptable excipient.

In some embodiments, the IGF-1-encoding DNA construct encodes Class I IGF-1Ea protein comprising a polypeptide of SEQ ID NO: 14 or Class I IGF-1Ec protein comprising a polypeptide of SEQ ID NO: 16.

In some embodiments, the IGF-1-encoding DNA construct encodes more than one human IGF-1 isoforms. In some embodiments, the IGF-1-encoding DNA construct encodes two human IGF-1 isoforms, wherein the two human IGF-1 isoforms are Class I IGF-1Ea protein comprising a polypeptide of SEQ ID NO: 14 and Class I IGF-1Ec protein comprising a polypeptide of SEQ ID NO: 16.

In some embodiments, the IGF-1-encoding DNA construct, comprising: a first IGF polynucleotide of SEQ ID NO: 1 (exons 1, 3, 4) or a degenerate thereof, a second IGF polynucleotide of SEQ ID NO: 2 (intron 4) or a fragment thereof, a third IGF polynucleotide of SEQ ID NO: 3 (exons 5 and 6-1) or a degenerate thereof, a fourth IGF polynucleotide of SEQ ID NO: 4 (intron 5) or a fragment thereof, and a fifth IGF polynucleotide of SEQ ID NO: 5 (exon 6-2) or a degenerate thereof, wherein the first polynucleotide, the second polynucleotide, the third polynucleotide, the fourth polynucleotide and the fifth polynucleotide are linked in sequential 5′ to 3′ order.

In some embodiments, the second IGF polynucleotide is a polynucleotide of SEQ ID NO: 6. In some embodiments, the second IGF polynucleotide is a polynucleotide of SEQ ID NO: 7. In some embodiments, the fourth IGF polynucleotide is a polynucleotide of SEQ ID NO: 8.

In some embodiments, the IGF-1-encoding DNA construct further comprises a plasmid vector. In some embodiments, the plasmid vector is pCK. In some embodiments, the IGF-1-encoding DNA construct is selected from the group consisting of pCK-IGF-1X6 and pCK-IGF-1X10. In some embodiments, the plasmid vector is pTx. In some embodiments, the IGF-1-encoding DNA construct is selected from the group consisting of pTx-IGF-1X6 and pTx-IGF-1X10.

In some embodiments, the IGF-1-encoding DNA construct comprises a polynucleotide of SEQ ID NO: 9. In some embodiments, the IGF-1-encoding DNA construct comprises a polynucleotide of SEQ ID NO: 10.

In some embodiments, the at least one human HGF isoform is flHGF of SEQ ID NO: 11 or dHGF of SEQ ID NO: 12. In some embodiments, the HGF-encoding DNA construct is capable of expressing both flHGF of SEQ ID NO: 11 and dHGF of SEQ ID NO: 12.

In some embodiments, the HGF-encoding DNA construct comprises: a first HGF polynucleotide of SEQ ID NO: 22 (exons 1-4) or a degenerate thereof, a second HGF polynucleotide of SEQ ID NO: 25 (intron 4) or a functional fragment thereof, and a third HGF polynucleotide of SEQ ID NO: 23 (exons 5-18) or a degenerate thereof, wherein the second HGF polynucleotide is located between the first HGF polynucleotide and the third HGF polynucleotide, and the first HGF-encoding DNA construct encodes two human HGF isoforms.

In some embodiments, the HGF-encoding DNA construct comprises a polynucleotide of any of SEQ ID Nos: 26-32 and 13. In some embodiments, the HGF-encoding DNA construct comprises a polynucleotide of SEQ ID NO: 13.

In some embodiments, the pharmaceutical composition comprises a polynucleotide of SEQ ID NO: 13; and a polynucleotide of SEQ ID NO: 9. In some embodiments, the pharmaceutical composition comprises a polynucleotide of SEQ ID NO: 13; and a polynucleotide of SEQ ID NO: 10. In some embodiments, the pharmaceutical composition comprises a polynucleotide of SEQ ID NO: 13; a polynucleotide of SEQ ID NO: 15 or a polynucleotide of SEQ ID NO: 17. In some embodiments, the pharmaceutical composition comprises a polynucleotide of SEQ ID NO: 13; a polynucleotide of SEQ ID NO: 15 and a polynucleotide of SEQ ID NO: 17.

In some embodiments, the pharmaceutical composition comprises a polynucleotide of SEQ ID NO: 33 and a polynucleotide of SEQ ID NO: 9, SEQ ID NO: 10, SEQ ID NO: 15, or SEQ ID NO: 17.

In yet another aspect, the present invention provides a kit for treating neuropathy, comprising: a first pharmaceutical composition comprising an IGF-1-encoding DNA construct capable of expressing at least one human IGF-1 isoform, and a first pharmaceutically acceptable excipient; and a second pharmaceutical composition comprising an HGF-encoding DNA construct capable of expressing at least one human HGF isoform, and a second pharmaceutically acceptable excipient.

In some embodiments, the IGF-1-encoding DNA construct encodes Class I IGF-1Ea protein comprising a polypeptide of SEQ ID NO: 14 or Class I IGF-1Ec protein comprising a polypeptide of SEQ ID NO: 16. In some embodiments, the IGF-1-encoding DNA construct encodes more than one human IGF-1 isoforms. In some embodiments, the IGF-1-encoding DNA construct encodes two human IGF-1 isoforms, wherein the two human IGF-1 isoforms are Class I IGF-1Ea protein comprising a polypeptide of SEQ ID NO: 14 and Class I IGF-1Ec protein comprising a polypeptide of SEQ ID NO: 16.

In some embodiments, the IGF-1-encoding DNA construct, comprising: a first IGF polynucleotide of SEQ ID NO: 1 (exons 1, 3, 4) or a degenerate thereof, a second IGF polynucleotide of SEQ ID NO: 2 (intron 4) or a fragment thereof, a third IGF polynucleotide of SEQ ID NO: 3 (exons 5 and 6-1) or a degenerate thereof, a fourth IGF polynucleotide of SEQ ID NO: 4 (intron 5) or a fragment thereof, and a fifth IGF polynucleotide of SEQ ID NO: 5 (exon 6-2) or a degenerate thereof, wherein the first polynucleotide, the second polynucleotide, the third polynucleotide, the fourth polynucleotide and the fifth polynucleotide are linked in sequential 5′ to 3′ order.

In some embodiments, the second IGF polynucleotide is a polynucleotide of SEQ ID NO: 6. In some embodiments, the second IGF polynucleotide is a polynucleotide of SEQ ID NO: 7. In some embodiments, the fourth IGF polynucleotide is a polynucleotide of SEQ ID NO: 8.

In some embodiments, the IGF-1-encoding DNA construct further comprises a plasmid vector. In some embodiments, the plasmid vector is pCK. In some embodiments, the IGF-1-encoding DNA construct comprises pCK-IGF-1X6 or pCK-IGF-1X10. In some embodiments, the plasmid vector is pTx. In some embodiments, the IGF-1-encoding DNA construct comprises pTx-IGF-1X6 or pTx-IGF-1X10.

In some embodiments, the IGF-1-encoding DNA construct comprises a polynucleotide of SEQ ID NO: 9. In some embodiments, the IGF-1-encoding DNA construct comprises a polynucleotide of SEQ ID NO: 10.

In some embodiments, the at least one human HGF isoform is flHGF of SEQ ID NO: 11 or dHGF of SEQ ID NO: 12. In some embodiments, the HGF-encoding DNA construct is capable of expressing both flHGF of SEQ ID NO: 11 and dHGF of SEQ ID NO: 12.

In some embodiments, the HGF-encoding DNA construct comprises: a first HGF polynucleotide of SEQ ID NO: 22 (exons 1-4) or a degenerate thereof, a second HGF polynucleotide of SEQ ID NO: 25 (intron 4) or a functional fragment thereof, and a third HGF polynucleotide of SEQ ID NO: 23 (exons 5-18) or a degenerate thereof, wherein the second HGF polynucleotide is located between the first HGF polynucleotide and the third HGF polynucleotide, and the first HGF-encoding DNA construct encodes two human HGF isoforms.

In some embodiments, the HGF-encoding DNA construct comprises a polynucleotide of any of SEQ ID Nos: 26-32 and 13. In some embodiments, the HGF-encoding DNA construct comprises a polynucleotide of SEQ ID NO: 13.

In some embodiments, the first pharmaceutical composition comprises a polynucleotide of SEQ ID NO: 9; and the second pharmaceutical composition comprises a polynucleotide of SEQ ID NO: 13.

In some embodiments, the first pharmaceutical composition comprises a polynucleotide of SEQ ID NO: 10; and the second pharmaceutical composition comprises a polynucleotide of SEQ ID NO: 13.

In some embodiments, the first pharmaceutical composition comprises a polynucleotide of SEQ ID NO:15 and a polynucleotide of SEQ ID NO: 17; and the second pharmaceutical composition comprises a polynucleotide of SEQ ID NO: 13.

In some embodiments, the first pharmaceutical composition comprises a polynucleotide of SEQ ID NO: 9, SEQ ID NO: 10, SEQ ID NO: 15, or SEQ ID NO: 17, and the second pharmaceutical composition comprises a polynucleotide of SEQ ID NO: 33.

In another aspect, the present disclosure provides a first IGF-1-encoding DNA construct capable of expressing a human IGF-1 isoform for use in the medical method of treating neuropathy, the medical method comprising the steps of: administering to a subject having neuropathy a therapeutically effective amount of the first IGF-1-encoding DNA construct, and administering to the subject a therapeutically effective amount of a first HGF-encoding DNA construct capable of expressing a human HGF isoform.

In some embodiments, the first IGF-1-encoding DNA construct is capable of expressing Class I IGF-1Ea protein comprising a polypeptide of SEQ ID NO: 14 or Class I IGF-1Ec protein comprising a polypeptide of SEQ ID NO: 16. In some embodiments, the first IGF-1-encoding DNA construct is not capable of expressing both Class II IGF-1 Ea protein comprising a polypeptide of SEQ ID NO: 18 and Class I IGF-1Eb protein comprising a polypeptide of SEQ ID NO: 20.

In some embodiments, the first IGF-1-encoding DNA construct comprises a polynucleotide of SEQ ID NO: 15. In some embodiments, the medical method further comprises the step of: administering to the subject a second IGF-1-encoding DNA construct, wherein the second IGF-1-encoding DNA construct comprises a polynucleotide of SEQ ID NO: 17.

In some embodiments, the first IGF-1-encoding DNA construct comprises a polynucleotide of SEQ ID NO: 17. In some embodiments, the medical method further comprises the step of: administering to the subject a second IGF-1-encoding DNA construct, wherein the second IGF-1-encoding DNA construct comprises a polynucleotide of SEQ ID NO: 15.

In some embodiments, the step of administering the first IGF-1-encoding DNA construct and the step of administering the second IGF-1-encoding DNA construct are performed concurrently. In some embodiments, the step of administering the first IGF-1-encoding DNA construct and the step of administering the second IGF-1-encoding DNA construct are performed sequentially.

In some embodiments, the first IGF-1-encoding DNA construct encodes more than one human IGF-1 isoforms. In some embodiments, the more than one human IGF-1 isoforms comprise a polypeptide of SEQ ID NO: 14 and a polypeptide of SEQ ID NO: 16.

In some embodiments, the first IGF-1-encoding DNA construct comprises: a first IGF polynucleotide of SEQ ID NO: 1 (exons 1, 3, 4) or a degenerate thereof, a second IGF polynucleotide of SEQ ID NO: 2 (intron 4) or a fragment thereof, a third IGF polynucleotide of SEQ ID NO: 3 (exons 5 and 6-1) or a degenerate thereof, a fourth IGF polynucleotide of SEQ ID NO: 4 (intron 5) or a fragment thereof, and a fifth IGF polynucleotide of SEQ ID NO: 5 (exon 6-2) or a degenerate thereof, wherein the first polynucleotide, the second polynucleotide, the third polynucleotide, the fourth polynucleotide and the fifth polynucleotide are linked in sequential 5′ to 3′ order.

In some embodiments, the second IGF polynucleotide is a polynucleotide of SEQ ID NO: 6. In some embodiments, the second IGF polynucleotide is a polynucleotide of SEQ ID NO: 7. In some embodiments, the fourth IGF polynucleotide is a polynucleotide of SEQ ID NO: 8.

In some embodiments, the first IGF-1-encoding DNA construct comprises a plasmid vector. In some embodiments, the plasmid vector is pCK. In some embodiments, the plasmid vector is pTx.

In some embodiments, the first IGF-1-encoding DNA construct comprises a polynucleotide of SEQ ID NO: 10. In some embodiments, the first IGF-1-encoding DNA construct comprises a polynucleotide of SEQ ID NO: 9.

In some embodiments, the first IGF-1-encoding DNA construct and the first HGF-encoding DNA construct are administered in an amount sufficient to reduce pain in the subject. In some embodiments, the subject has diabetic neuropathy. In some embodiments, the first IGF-1-encoding DNA construct and the first HGF-encoding DNA construct are administered by a plurality of intramuscular injections.

In some embodiments, the human HGF isoform is flHGF of SEQ ID NO: 11 or dHGF of SEQ ID NO: 12. In some embodiments, the first HGF-encoding DNA construct encodes more than one human HGF isoforms. In some embodiments, the first HGF-encoding DNA construct encodes two human HGF isoforms, wherein the two human HGF isoforms are flHGF of SEQ ID NO: 11 and dHGF of SEQ ID NO: 12.

In some embodiments, the first HGF-encoding DNA construct comprises a plasmid vector, optionally wherein the plasmid vector is a pCK vector or a pTx vector. In some embodiments, the first HGF-encoding DNA construct comprises: a first HGF polynucleotide of SEQ ID NO: 22 (exons 1-4) or a degenerate thereof, a second HGF polynucleotide of SEQ ID NO: 25 (intron 4) or a functional fragment thereof, and a third HGF polynucleotide of SEQ ID NO: 23 (exons 5-18) or a degenerate thereof, wherein the second HGF polynucleotide is located between the first HGF polynucleotide and the third HGF polynucleotide, and the first HGF-encoding DNA construct encodes two human HGF isoforms.

In some embodiments, the first HGF-encoding DNA construct comprises a polynucleotide of SEQ ID NO: 13.

In some embodiments, the first IGF-1-encoding DNA construct and the first HGF-encoding DNA construct are co-administered. In some embodiments, the first IGF-1-encoding DNA construct and the first HGF-encoding DNA construct are co-administered by an intramuscular injection. In some embodiments, the step of administering the first IGF-1-encoding DNA construct and the step of administering the first HGF-encoding DNA construct are performed separately. In some embodiments, the step of administering the first IGF-1-encoding DNA construct and the step of administering the first HGF-encoding DNA construct are performed at least three weeks apart.

In some embodiments, the medical method further comprises the step of administering to the subject a second HGF-encoding DNA construct capable of expressing a human HGF isoform selected from flHGF of SEQ ID NO: 11 and dHGF of SEQ ID NO: 12.

In another aspect, the present disclosure further provides a first HGF-encoding DNA construct capable of expressing a human HGF isoform for use in the medical method of treating neuropathy, the methodical method comprising the steps of: administering to a subject having neuropathy a therapeutically effective amount of the first HGF-encoding DNA construct, and administering to the subject a therapeutically effective amount of the IGF-1-encoding DNA construct capable of expressing a human IGF-1 isoform.

### 5. BRIEF DESCRIPTION OF THE DRAWINGS

FIG. 1 is a schematic representation of the human IGF-1 gene including alternative transcription initiation sites and alternative splicing sites. IGF-1 isoforms that are naturally produced from the IGF-1 gene include Class I Ec (Isoform #1); Class II Ea (Isoform #2); Class I Eb (Isoform #3); and Class I Ea (Isoform #4).

FIG. 2A outlines the experimental protocol for testing therapeutic efficacy of concurrent administration of an HGF-encoding DNA construct (VM202) and a DNA construct encoding a single IGF-1 isoform in the chronic constriction injury (CCI) model.

FIG. 2B is a graph showing the frequency (%) of paw withdrawal measured in CCI mice or in sham mice in the experiment outlined in FIG. 2A. The CCI mice were injected with a DNA construct—(i) pCK vector (“pCK”), (ii) VM202 (“VM202”), or (iii) VM202 plus (+) an IGF-1-encoding DNA construct—VM202 and pCK-IGF-1 #1 (“1”), VM202 and pCK-IGF-1 #2 (“2”), VM202 and pCK-IGF-1 #3 (“3”), or VM202 and pCK-IGF-1 #4 (“4”).

FIG. 3A outlines the experimental protocol for testing therapeutic efficacy of concurrent administration of an HGF-encoding DNA construct (VM202) and one or two DNA constructs encoding a single IGF-1 isoform, in the chronic constriction injury (CCI) model.

FIG. 3B is a graph showing the frequency (%) of paw withdrawal measured in CCI mice or sham mice in the experiment outlined in FIG. 3A. The CCI mice were injected with a DNA construct—(i) pCK vector (“pCK”), (ii) VM202 (“VM202”), or (iii) VM202 and an IGF-1-encoding DNA construct—VM202 and pCK-IGF-1 #1 (“1”), VM202 and pCK-IGF-1 #4 (“4”) or VM202, pCK-IGF-1 #1 and pCK-IGF-1 #4 (“1+4”).

FIG. 4A outlines the experimental protocol for testing therapeutic efficacy of serial administration of an HGF-encoding DNA construct (VM202) and two IGF-1-encoding DNA constructs, pCK-IGF-1 #1 and pCK-IGF-1 #4, in the chronic constriction injury (CCI) model.

FIG. 4B is a graph showing the frequency (%) of paw withdrawal measured in CCI mice in the experiment outlined in FIG. 4A. The CCI mice were injected twice with one or more DNA constructs—(i) pCK vector in the 1^{st} injection and pCK vector in the 2^{nd} injection (“pCK”), (ii) pCK-IGF-1 #1 and pCK-IGF-1 #4 in the 1^{st} injection and pCK-IGF-1 #1 and pCK-IGF-1 #4 in the 2^{nd} injection (“IGF-1->IGF-1”), (iii) VM202 in the 1^{st} injection and pCK vector in the 2^{nd}injection (“VM202->pCK”), (iv) pCK-IGF-1 #1 and pCK-IGF-1 #4 in the 1^{st} injection and pCK vector in the 2^{nd} injection (“IGF-1->pCK”), (v) pCK-IGF-1 #1 and pCK-IGF-1 #4 in the 1^{st} injection and VM202 in the 2^{nd} injection (“IGF-1->VM202”), (vi) VM202 in the 1^{st} injection and VM202 in the 2^{nd} injection (“VM202->VM202”), or (vii) VM202 in the 1^{st} injection and pCK-IGF-1 #1 and pCK-IGF-1 #4 in the 2^{nd} injection (“VM202->IGF-1 isoforms”).

FIG. 5A outlines the experimental protocol used in Example 3 to assess in vivo expression of IGF-1 isoforms from various DNA constructs.

FIG. 5B shows results of an ELISA measuring the amount of total human IGF-1 isoforms expressed after injection of a DNA construct encoding no IGF (vector only, “pCK”); pCK-IGF-1 #1 (“1”); pCK-IGF-1 #4 (“4”); pCK-IGF-1 #1 and pCK-IGF-1 #4 (“1+4”); and a dual expression constructs pCK-IGF-1X6 (“X6”) and pCK-IGF-1X10 (“X10”).

FIG. 6A shows the location of forward (“F”) and reverse (“R”) primers used in RT-PCR for discriminating the expression of IGF-1 isoforms #1 (Class I Ec isoform) and #4 (Class I Ea isoform).

FIG. 6B shows agarose gel electrophoresis of RT-PCR products, showing expression of isoforms #1 and #4 from dual expression constructs pCK-IGF-1X6 and pCK-IGF-1X10. Both pCK-IGF-1X6 and pCK-IGF-1X10 induced high-level expression of both isoforms.

FIG. 7A outlines the protocol used in Example 3 to assess protein expression from the IGF-1-encoding DNA constructs in 293T cells in vitro.

FIG. 7B shows western blotting results demonstrating expression of IGF-1 isoforms #1 and/or #4 after in vitro transfection of (i) pCK-IGF-1 #1 (“1”), (ii) pCK-IGF-1 #4 (“4”), (iii) two single expression constructs, pCK-IGF-1 #1 and pCK-IGF-1 #4 (“1+4”), (iv) a dual expression construct pCK-IGF-1X6 (“X6”), or (v) a dual expression construct pCK-IGF-1X10 (“X10”).

FIG. 8A outlines the experimental protocol used in Example 4 to test efficacy of concurrent administration of the HGF-encoding construct, VM202, and various IGF-1-encoding DNA constructs in reducing mechanical allodynia in the CCI animal model.

FIG. 8B is a graph showing the frequency of paw withdrawal measured in the sham mice or CCI mice in the experiment outlined in FIG. 8A. The CCI mice were injected with one or more DNA constructs—(i) pCK vector (“pCK”), (ii) VM202 (“VM202”), (iii) VM202, pCK-IGF-1 #1, and pCK-IGF-1 #4 (“IGF-1 #1+#4”), (iv) VM202 and pCK-IGF-1X6 (“IGF-1X6”) and (v) VM202 and pCK-IGF-1X10 (“IGF-1X10”).

FIG. 9A outlines the experimental protocol used in Example 5 to test efficacy of concurrent administration of a construct expressing HGF₇₂₈ and various IGF-1-encoding DNA constructs in reducing mechanical allodynia in the CCI animal model.

FIG. 9B is a graph showing the frequency of paw withdrawal measured in the sham mice or CCI mice in the experiment outlined in FIG. 9A.

FIG. 9C is a graph showing the threshold of paw withdrawal measured in the Sham mice or CCI mice in the experiment outlined in FIG. 9A. The CCI mice were injected with one or more DNA constructs—vector alone (“CCI-pCK”), or (i) pCK-HGF₇₂₈ (“CCI-HGF₇₂₈”), (ii) pCK-HGF₇₂₈ and pCK-IGF-1 #1 (“CCI-HGF₇₂₈+IGF-1 #1”), (iii) pCK-HGF₇₂₈ and pCK-IGF-1 #4 (“CCI-HGF₇₂₈+IGF-1 #4”), or (iv) pCK-HGF₇₂₈ and pCK-IGF-1X10 (“CCI-HGF₇₂₈+IGF-1X10”).

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

The terms “isoform of IGF-1,” “human IGF-1 isoform” or “IGF-1 isoform” as used herein are used interchangeably herein to refer to a polypeptide having an amino acid sequence that is at least 80% identical to the amino acid sequence of one of naturally occurring pre-pro-IGF-1 polypeptides of humans, or their allelic variant, splice variant, or deletion variant. The naturally occurring pre-pro-IGF-1 polypeptides include Class I, Ec (SEQ ID NO: 16); Class II, Ea (SEQ ID NO: 18); Class I, Eb (SEQ ID NO: 20); and Class I, Ea isoforms (SEQ ID NO: 14).

The terms “Isoform #1,” “Class I, Ec isoform,” “Class I, IGF-1 Ec isoform” or “Class I, IGF-1 Ec” are used interchangeably herein to refer to a polypeptide of SEQ ID NO: 16.

The terms “Isoform #2,” “Class II, Ea isoform,” “Class II, IGF-1 Ea isoform” or “Class II, IGF-1 Ea” are used interchangeably herein to refer to a polypeptide of SEQ ID NO: 18.

The terms “Isoform #3,” “Class I, Eb isoform,” “Class I, IGF-1 Eb isoform” or “Class I, IGF-1 Eb” are used interchangeably herein to refer to a polypeptide of SEQ ID NO: 20.

The terms “Isoform #4,” “Class I, Ea isoform,” “Class I, IGF-1 Ea isoform” or “Class I, IGF-1 Ea” are used interchangeably herein to refer to a polypeptide of SEQ ID NO: 14.

The term “treatment” as used herein refers to all the acts of (a) suppression a symptom of neuropathy; (b) alleviation of a symptom of neuropathy; and (c) removal of a symptom of neuropathy. In some embodiments, the composition of the present invention can treat neuropathy through the growth of neuronal cells or the suppression of neuronal cell death.

The term “VM202” as used herein refers to a plasmid DNA also called as pCK-HGF-X7, comprising pCK vector (SEQ ID NO: 24) and HGF-X7 (SEQ ID NO: 13) cloned into the pCK vector. VM202 was deposited under the terms of the Budapest Treaty at the Korean Culture Center of Microorganisms (KCCM) under accession number KCCM-10361 on Mar. 12, 2002.

The term “isoforms of HGF” as used herein refers to a polypeptide having an amino acid sequence that is at least 80% identical to the amino acid sequence of a naturally occurring HGF polypeptide in an animal, including humans. The term includes polypeptides having an amino acid sequence that is at least 80% identical to any full length wild type HGF polypeptide, and includes polypeptides having an amino acid sequence that is at least 80% identical to a naturally occurring HGF allelic variant, splice variant, or deletion variant. Isoforms of HGF preferred for use in the present invention include two or more isoforms selected from the group consisting of full-length HGF (flHGF) (synonymously, fHGF), deleted variant HGF (dHGF), NK1, NK2, and NK4. According to a more preferred embodiment of the present invention, the isoforms of HGF used in the methods described herein include flHGF (SEQ ID NO: 11) and dHGF (SEQ ID NO: 12).

The terms “human flHGF”, “flHGF” and “fHGF” are used interchangeably herein to refer to a protein consisting of amino acids 1-728 of the human HGF protein. The sequence of flHGF is provided in SEQ ID NO: 11.

The terms “human dHGF” and “dHGF” are used interchangeably herein to refer to a deleted variant of the HGF protein produced by alternative splicing of the human HGF gene. Specifically, “human dHGF” or “dHGF” refers to a human HGF protein with deletion of five amino acids (F, L, P, S, and S) in the first kringle domain of the alpha chain from the full length HGF sequence. Human dHGF is 723 amino acids in length. The amino acid sequence of human dHGF is provided in SEQ ID NO: 12.

The term “therapeutically effective dose” or “effective amount” as used herein refers to a dose or an amount that produces the desired effect for which it is administered. In the context of the present methods, a therapeutically effective amount is an amount effective to treat a symptom of neuropathy. The amount can be an amount effective to treat a symptom of neuropathy by itself or in combination with other therapeutic agent.

The term “sufficient amount” as used herein refers to an amount sufficient to produce a desired effect. The amount can be an amount sufficient to produce desired effect by itself or in combination with other therapeutic agent.

The term “degenerate sequence” as used herein refers to a nucleic acid sequence that can be translated to provide an amino acid sequence identical to that translated from the reference nucleic acid sequence.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50

### 6.3. Methods of Treating Neuropathy

In a first aspect, methods are provided for treating neuropathy. The methods comprise administering to a subject having neuropathy a therapeutically effective amount of a first IGF-1-encoding DNA construct capable of expressing a human IGF-1 isoform; and a therapeutically effective amount of first HGF-encoding DNA construct capable of expressing a human HGF isoform.

### 6.3.1. IGF-1-Encoding DNA Constructs

In the methods provided herein, DNA constructs capable of expressing at least one isoform of human IGF-1 are used.

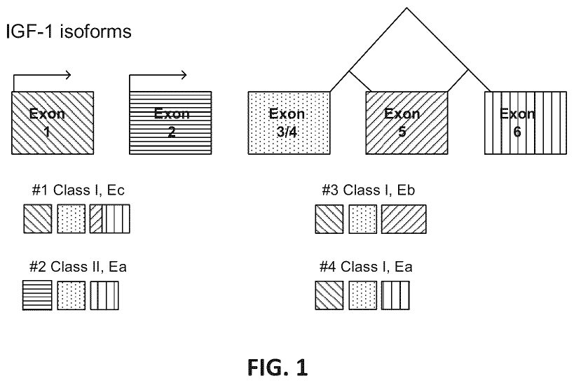

As illustrated in FIG. 1, the human IGF-1 gene contains six exons (exons 1, 2, 3, 4, 5, and 6 (6-1 and 6-2)) spanning nearly 90 kb of genomic DNA. Exons 1 and 2 are mutually exclusive leader exons, each having multiple promoter sites that are variably used. Further, the IGF-1 gene can be differentially spliced to create multiple transcript variants. Each transcript variant encodes a different pre-pro-IGF-1 protein (“IGF-1 isoform”) possessing variable signaling peptide leader sequences. Yet all the transcript isoforms give rise to the same mature 70-amino acid IGF-1 peptide that uses the same receptor after processing.

The pre-pro-IGF-1 peptides differ in their leader, or signal, sequences and in their carboxy (C)-terminus. Incorporation of exon 1 or exon 2 is mutually exclusive and one of them serves as a leader sequence of the pre-pro-IGF-1 peptide; the different leader exons create different 5′-UTRs. The pre-pro-IGF-1 polypeptides undergo posttranscriptional proteolytic cleavage to remove the leader and the E-peptide carboxy-terminus giving rise to the mature 70-amino acid IGF-1.

Transcripts containing exon 1 are referred to as Class 1 transcripts (e.g., Class I, Ec; Class I, Eb; and Class I, Ea in FIG. 1) whereas those containing exon 2 are referred to as Class 2 transcripts (e.g., Class II, Ea in FIG. 1). Nearly all pre-pro peptides include 27 amino acids in the signaling peptide derived from exon 3 with the remaining signal sequences derived from the inclusion of exon 1 or 2. A minority of transcripts utilize a different transcription initiation site within exon 3 generating a shorter signaling peptide of 22 amino acids. Exons 3 and 4 are invariant and encode the B, C, A, and D domains of the mature IGF-1 peptide; exon 4 encodes two thirds of the mature IGF-1 peptide. The human Eb peptide is composed of only exons 4 and 5 whereas Ec contains exons 4, 5, and 6 (FIG. 1).

Alternative splicing and mutually exclusive initiation of transcription are illustrated in FIG. 1 that result in generation of different pre-pro-IGF-1 polypeptides (i.e., IGF-1 isoforms). Specifically, Class I, Ec IGF-1 isoform (SEQ ID NO: 16), comprising at least a fragment of exons 1, 3/4, 5 and 6, is generated from a transcript comprising a sequence of SEQ ID NO: 17. Class II, Ea IGF-1 isoform (SEQ ID NO:18), comprising at least a fragment of exons 2, 3/4 and 6, is generated from a transcript comprising a sequence of SEQ ID NO:19. Class I, Eb IGF-1 isoform (SEQ ID NO:20), comprising at least a fragment of exons 1, 3/4 and 5, is generated from a transcript comprising a sequence of SEQ ID NO:21. Class I, Ea IGF-1 isoform (SEQ ID NO:14), comprising at least a fragment of exons 1, 3/4 and 6 are generated from a transcript comprising a sequence of SEQ ID NO: 15.

Although the mature IGF-1 protein derived from the various transcripts does not differ, the various transcript isoforms have been suggested to have different regulatory roles. The variant forms possess different stabilities, binding partners, and activity indicating a pivotal regulatory role for the isoforms. The biological significance of the isoforms remains unclear, although it has been hypothesized that Class I isoforms with exon 1 are autocrine/paracrine forms while Class II isoforms with exon 2 are secreted endocrine forms. This is based on the finding that Class II transcripts include a typical signal peptide motif associated with efficient secretion, whereas Class I transcripts have a longer signal peptide that can possibly interfere with secretion.

Most tissues are believed to use Class I transcripts, although liver uses both forms and hepatic Class II transcripts are preferentially enhanced during development. There are many changes in the IGF-1 transcript abundance during development. It was found that Class 1, Ea is the most abundant form during the active growth phase and Class 1, Eb is also expressed uniformly, albeit at lower levels, across the growth plate during early growth phases.

DNA constructs capable of expressing at least one isoform of human IGF-1 are provided herein. Such single expression construct includes, but is not limited to, pCK-IGF-1 #1 which is a pCK vector containing a coding sequence for IGF-1 isoform #1; pCK-IGF-1 #2 which is a pCK vector containing a coding sequence for IGF-1 isoform #2; pCK-IGF-1 #3 which is a pCK vector containing a coding sequence for IGF-1 isoform #3, and pCK-IGF-1 #4 which is a pCK vector containing a coding sequence for IGF-1 isoform #4. In some embodiments, more than one DNA constructs, each encoding a different IGF-1 isoform, are used. For example, a first construct encoding Class I, Ec isoform (Isoform #1) and a second construct encoding Class I, Ea isoform (Isoform #4) are used together. For example, pCK-IGF-1 #1 and pCK-IGF-1 #4 can be used together.

Such single expression construct further includes, but is not limited to, pTx-IGF-1 #1 which is a pTx vector containing a coding sequence for IGF-1 isoform #1; pTx-IGF-1 #2 which is a pTx vector containing a coding sequence for IGF-1 isoform #2; pTx-IGF-1 #3 which is a pTx vector containing a coding sequence for IGF-1 isoform #3, and pTx-IGF-1 #4 which is a pTx vector containing a coding sequence for IGF-1 isoform #4. In some embodiments, more than one DNA constructs, each encoding a different IGF-1 isoform, are used. For example, a first construct encoding Class I, Ec isoform (Isoform #1) and a second construct encoding Class I, Ea isoform (Isoform #4) are used together. For example, pTx-IGF-1 #1 and pTx-IGF-1 #4 can be used together.

In some embodiments, a DNA construct that expresses two or more isoforms (i.e., “a dual expression construct”) is used. For example, a single DNA construct encoding both Class I, Ec isoform and Class I, Ea isoform can be used.

In some embodiments, the DNA construct contains a coding sequence of one of the IGF-1 isoforms. For example, the DNA construct can comprise a sequence encoding Class I, Ea (Isoform #4) (SEQ ID NO: 15); Class I, Eb (Isoform #3) (SEQ ID NO:21); Class I, Ec (Isoform #1) (SEQ ID NO:17); or Class II, Ea (Isoform #2) (SEQ ID NO:19).

In some embodiments, the DNA construct is a dual expression construct, a DNA construct that can express more than one IGF-1 isoforms, by comprising an expression regulatory sequence for each isoform coding sequence (CDS). In some embodiments, the construct comprises an internal ribosomal entry site (IRES) between two coding sequences, for example, in the order of (1) expression regulatory sequence-(2) coding sequence of first isoform-(3) IRES-(4) coding sequence of second isoform-(5) transcription termination sequence. IRES allows translation to start at the IRES sequence, thereby allowing expression of two protein products from a single transcript. In yet further embodiments, a plurality of constructs, each encoding a single isoform of IGF-1, are used together to induce expression of more than one isoforms of IGF-1 in the subject to whom administered.

In preferred embodiments, the DNA construct is capable of expressing two or more IGF-1 isoforms simultaneously—e.g., (i) Class I, Ec isoform (Isoform #1) and Class II, Ea isoform (Isoform #2); (ii) Class I, Ec isoform (Isoform #1) and Class I, Eb isoform (Isoform #3); (iii) Class I, Ec isoform (Isoform #1) and Class I, Ea isoform (Isoform #4); (iv) Class II, Ea isoform (Isoform #2) and Class I, Eb isoform (Isoform #3); (v) Class II, Ea isoform (Isoform #2) and Class I, Ea isoform (Isoform #4); (vi) Class I, Eb isoform (Isoform #3) and Class I, Ea isoform (Isoform #4)—by comprising an alternative splicing site.

For example, the DNA construct can comprise (i) a first sequence comprising exons 1, 3 and 4 of a human IGF-1 gene (SEQ ID NO:1) or a degenerate sequence of the first sequence; (ii) a second sequence comprising intron 4 of the human IGF-1 gene (SEQ ID NO:2) or a fragment of the second sequence; (iii) a third sequence comprising exons 5 and 6-1 of the human IGF-1 gene (SEQ ID NO:3) or a degenerate sequence of the third sequence; (iv) a fourth sequence comprising intron 5 of the human IGF-1 gene (SEQ ID NO:4) or a fragment of the second sequence; and (v) a fifth sequence comprising exon 6-2 of the human IGF-1 gene (SEQ ID NO:5) or a degenerate sequence of the fifth sequence. Introns 4 and 5 can be alternatively spliced, resulting in production of two isoforms of IGF-1 (e.g., Class I, Ec and Class I, Ea).

In some embodiments, the DNA construct is tested in vitro and/or in vivo related to its capability to express one or more IGF-1 isoforms. In preferred embodiments, DNA constructs capable of expressing both Class I, Ec and Class I, Ea IGF-1 isoforms are selected.

In some embodiments, the construct comprises a full sequence of intron 4 (SEQ ID NO:2) or its fragment. In preferred embodiments, the construct comprises a fragment of intron 4 having a sequence of SEQ ID NO: 6 or SEQ ID NO: 7.

In some embodiments, the construct comprises a full sequence of intron 5 (SEQ ID NO: 4), or its fragment. In preferred embodiments, the construct comprises a fragment of intron 5 having a sequence of SEQ ID NO: 8.

Various DNA constructs comprising sequences corresponding (i) exons 1-6 of the human IGF-1 gene and (ii) introns 4 and 5 of the human IGF-1 gene or various fragments of introns 4 and 5 are named “IGF-1X” followed by a unique number. The IGF-1X constructs tested by Applicant include, but are not limited to, IGF-1X1, IGF-1X2, IGF-1X3, IGF-1X4, IGF-1X5, IGF-1X6, IGF-1X7, IGF-1X8, IGF-1X9 and IGF-1X10. The IGF-1X constructs cloned in a pCK vector are referred to as pCK-IGF-1X1, pCK-IGF-1X2, pCK-IGF-1X3, pCK-IGF-1X4, pCK-IGF-1X5, pCK-IGF-1X6, pCK-IGF-1X7, pCK-IGF-1X8, pCK-IGF-1X9 and pCK-IGF-1X10, respectively. Among the tested constructs, pCK-IGF-1X6 and pCK-IGF-1X10 were identified to express both Class I, Ec and Class I, Ea IGF-1 isoforms. The IGF-1X constructs cloned in a pTx vector are referred to as pTx-IGF-1X1, pTx-IGF-1X2, pTx-IGF-1X3, pTx-IGF-1X4, pTx-IGF-1X5, pTx-IGF-1X6, pTx-IGF-1X7, pTx-IGF-1X8, pTx-IGF-1X9 and pTx-IGF-1X10, respectively. pTx-IGF-1X6 and pTx-IGF-1X10 express both Class I, Ec and Class I, Ea IGF-1 isoforms.

In preferred embodiments, IGF-1X6 (SEQ ID NO:9) or IGF-1X10 (SEQ ID NO:10) is used. IGF-1X6 (SEQ ID NO:9) and IGF-1X10 (SEQ ID NO:10) cloned into a pCK vector are named pCK-IGF-1X6 and pCK-IGF-1X10, respectively. E. coli cells transformed with pCK-IGF-1X6 (“DH5α_pCK-IGF1X6”) were deposited under the terms of the Budapest Treaty at the Korea Collection for Type Cultures (KCTC, Korea Research Institute of Bioscience and Biotechnology (KRIBB) 181, Ipsin-gil, Jeongeup-si, Jeollabuk-do, 56212, Republic of Korea) with accession number KCTC 13539BP on May 30, 2018. E. coli cells transformed with pCK-IGF-1X10 (“DH5α_pCK-IGF1 X10”) were deposited under the terms of the Budapest Treaty at the Korea Collection for Type Cultures (KCTC, Korea Research Institute of Bioscience and Biotechnology (KRIBB) 181, Ipsin-gil, Jeongeup-si, Jeollabuk-do, 56212, Republic of Korea) with accession number KCTC 13540BP on May 30, 2018.

In other preferred embodiments, IGF-1X6 (SEQ ID NO:9) and IGF-1X10 (SEQ ID NO:10) cloned into a pTx vector (SEQ ID NO: 38) are used. The IGF constructs are named pTx-IGF-1X6 and pTx-IGF-1X10 (SEQ ID NO: 39), respectively.

IGF-1 isoforms or DNA constructs encoding IGF-1 isoforms described herein can include modifications from the wild type human IGF-1 isoforms. The modified sequences can include sequences with at least 80% identity, more preferably at least 90% identity and most preferably at least 95% identity when the modified sequences are aligned with the wild type human IGF-1 isoform sequences in the maximal manner. Methods of alignment of sequences for comparison are well-known in the art. Specifically, alignment algorithm disclosed in the NCBI Basic Local Alignment Search Tool (BLAST) of the National Center for Biological Information (NBCl, Bethesda, Md.) website and used in connection with the sequence analysis programs blastp, blasm, blastx, tblastn and tblastx can be used to determine the percent identity.

### 6.3.2. HGF-Encoding DNA Constructs

In the methods provided herein, DNA constructs capable of expressing at least one isoform of human HGF are used.

Hepatocyte growth factor (HGF) is a heparin-binding glycoprotein also known as scatter factor or hepatopoietin-A. HGF has multiple biological effects such as mitogenesis, motogenesis, and morphogenesis of various cell types. HGF is encoded by a gene containing 18 exons and 17 introns, located at chromosome 7q21.1.

The HGF gene encodes two isoforms of HGF by an alternative splicing between exon 4 and exon 5—the two isoforms include: (1) a full-length polypeptide HGF precursor (“flHGF”) containing 728 amino acids (SEQ ID NO:11) with the following domains: N-terminal hairpin loop-kringle 1-kringle 2-kringle 3-kringle 4-inactivated serine protease and (2) deleted variant HGF (“dHGF”) containing 723 amino acids (SEQ ID NO: 12) with deletion of five amino acids in the first kringle domain of the alpha chain (i.e., F, L, P, S and S). flHGF and dHGF share several biological functions, but differ in terms of immunological characteristics and several biological properties. It has been demonstrated that these two isoforms of HGF are effective in treating diabetic neuropathy, as disclosed in U.S. Pub. No. 20140296142 incorporated by reference by its entirety herein.

Some embodiments of the present invention provide a method of administering a construct encoding one or more isoforms of HGF. In some embodiments, a construct encoding both flHGF and dHGF is used. In some embodiments, a construct encoding either flHGF or dHGF is used. Specifically, a construct comprising a polynucleotide of SEQ ID NO: 33 can be used. The constructs can comprise a vector with one or more regulatory sequences (e.g., a promoter or an enhancer) operatively linked to a coding sequence encoding flHGF, dHGF, or both. The regulatory sequence can regulate expression of the HGF isoform.

In some embodiments, a construct can encode two or more isoforms of HGF by comprising an expression regulatory sequence for coding sequence (CDS) of each isoform. Alternatively, the construct can comprise an internal ribosomal entry site (IRES) between two coding sequences, for example, in the order of (1) expression regulatory sequence-(2) coding sequence of first isoform-(3) IRES-(4) coding sequence of second isoform-(5) transcription termination sequence. IRES allows translation to start at the IRES sequence, thereby allowing expression of two genes of interest from a single construct. Alternatively, more than one constructs, each encoding a single isoform of HGF, can be used together to induce expression of more than one isoforms of HGF in the target.

In preferred embodiments, a construct is used that simultaneously expresses two or more different isoforms of HGF—i.e., flHGF and dHGF—by comprising an alternative splicing site. It was previously demonstrated in U.S. Pat. No. 7,812,146, incorporated by reference in its entirety herein, that a construct encoding two isoforms of HGF (flHGF and dHGF) has much higher (almost 250 fold higher) expression efficiency than a construct encoding one isoform of HGF (either flHGF or dHGF).

The construct can include cDNA corresponding exon 1-18 of human HGF and intron 4 of a human HGF gene or its fragment, which is inserted between exon 4 and exon 5 of the cDNA. From the construct, two isoforms of HGF (flHGF and dHGF) can be generated by alternative splicing between exon 4 and exon 5. In some embodiments, the construct comprises a full sequence of intron 4 (SEQ ID NO:25). In some embodiments, the construct comprises a fragment of intron 4.

The construct comprising cDNA corresponding exon 1-18 of human HGF and intron 4 of a human HGF gene or its fragment can encode two isoforms of HGF by alternative splicing in intron 4 or its fragment. Specifically, the construct can comprise a nucleotide sequence selected from the group consisting of SEQ ID NO: 13 and SEQ ID NO: 26 to SEQ ID NO: 32. The nucleotide sequence of SEQ ID NO: 26 is 7113 bp and corresponds to a construct comprising the full sequence of intron 4. The nucleotide sequence of SEQ ID NOS: 13 and 27-32 correspond to constructs comprising various fragments of intron 4.

Various DNA constructs comprising cDNA corresponding exon 1-18 of human HGF and intron 4 of a human HGF gene or its fragment are named “HGF-X” followed by a unique number. The HGF-X that can be used for various embodiments of the present invention includes, but not limited to, HGF-X1 (SEQ ID NO: 26), HGF-X2 (SEQ ID NO: 27), HGF-X3 (SEQ ID NO: 28), HGF-X4 (SEQ ID NO: 29), HGF-X5 (SEQ ID NO: 30), HGF-X6 (SEQ ID NO: 31), HGF-X7 (SEQ ID NO: 13; HGF coding sequence in VM202), and HGF-X8 (SEQ ID NO: 32).

pCK-HGF-X7 (i.e., VM202) was demonstrated to have the highest expression efficiency as disclosed in U.S. Pat. No. 7,812,146. Accordingly, a DNA construct comprising HGF-X7 can be used in preferred embodiments of the present invention.

The constructs used in this invention may include nucleotide sequences substantially identical to sequences of the wild type human HGF isoforms. The substantial identity includes sequences with at least 80% identity, more preferably at least 90% identity and most preferably at least 95% identity as measured using one of the sequence comparison algorithms where the amino acid sequence or nucleotide sequence of the wild type human HGF isoform is aligned with a sequence in the maximal manner. Methods of alignment of sequences for comparison are well-known in the art. Various programs and alignment algorithms are described in: Smith and Waterman, Adv. Appl. Math. 2: 482 (1981); Needleman and Wunsch, J. Mol. Bio. 48: 443 (1970); Pearson and Lipman, Methods in Mol. Biol. 24: 307-31 (1988); Higgins and Sharp, Gene 73: 15 237-44 (1988); Higgins and Sharp, CABIOS 5: 151-3 (1989) Corpet et al., Nuc. Acids Res. 16: 10881-90 (1988); Huang et al., Comp. Appl. BioSci. 8: 155-65 (1992); and Pearson et al., Meth. Mol. Biol. 24: 307-31 (1994). The NCBI Basic Local Alignment Search Tool (BLAST) [Altschul 20 et al., J. Mol. Biol. 215: 403-10 (1990) J is available from several sources, including the National Center for Biological Information (NBCl, Bethesda, Md.) and on the Internet, for use in connection with the sequence analysis programs blastp, blasm, blastx, tblastn and tblastx.

### 6.3.3. Vector

DNA constructs expressing an IGF-1 isoform or an HGF isoform used in the methods described herein typically comprise a vector with one or more regulatory sequences (e.g., a promoter or an enhancer) operatively linked to the expressed sequences. The regulatory sequence regulates expression of the isoforms of IGF-1 or the isoforms of HGF.

It is preferred that the polynucleotide encoding one or more IGF-1 isoforms or HGF isoforms is operatively linked to a promoter in an expression construct. The term “operatively linked” refers to a functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

In typical embodiments, the promoter linked to the polynucleotide is operable in, preferably, animals, more preferably, mammalian cells, to control transcription of the polynucleotide, including the promoters derived from the genome of mammalian cells or from mammalian viruses, for example, CMV (cytomegalovirus) promoter, the adenovirus late promoter, the vaccinia virus 7.5K promoter, SV40 promoter, HSV tk promoter, RSV promoter, EF1 alpha promoter, metallothionein promoter, beta-actin promoter, human IL-2 gene promoter, human IFN gene promoter, human IL-4 gene promoter, human lymphotoxin gene promoter and human GM-CSF gene promoter, but not limited to. More preferably, the promoter useful in this invention is a promoter derived from the IE (immediately early) gene of human CMV (hCMV) or EF1 alpha promoter, most preferably hCMV IE gene-derived promoter/enhancer and 5′-UTR (untranslated region) comprising the overall sequence of exon 1 and exon 2 sequence spanning a sequence immediately before the ATG start codon.

The expression cassette used in this invention can comprise a polyadenylation sequence, for example, including bovine growth hormone terminator (Gimmi, E. R., et al., Nucleic Acids Res. 17:6983-6998 (1989)), SV40-derived polyadenylation sequence (Schek, N, et al., Mol. Cell Biol. 12:5386-5393 (1992)), HIV-1 polyA (Klasens, B. I. F., et al., Nucleic Acids Res. 26:1870-1876 (1998)), β-globin polyA (Gil, A., et al, Cell 49:399-406 (1987)), HSV TK polyA (Cole, C. N. and T. P. Stacy, Mol. Cell. 5 Biol. 5: 2104-2113 (1985)) or polyoma virus polyA (Batt, D. Band G. G. Carmichael, Mol. Cell. Biol. 15:4783-4790 (1995)), but not limited thereto.

### 6.3.3.1. Non-Viral Vector

In some embodiments, the IGF-1-encoding DNA construct capable of expressing a human IGF-1 isoform and/or the HGF-encoding DNA construct capable of expressing a human HGF isoform is a non-viral vector capable of expressing one or more IGF-1 isoforms or one or more HGF isoforms.

In typical embodiments, the non-viral vector is a plasmid. In currently preferred embodiments, the plasmid is pCK, pCP, pVAX1, pTx or pCY. In particularly preferred embodiments, the plasmid is pCK, details of which can be found in WO 2000/040737 and Lee et al., Biochem. Biophys. Res. Comm. 272:230-235 (2000), both of which are incorporated herein by reference in their entireties. E. coli transformed with pCK (Top10-pCK) was deposited at the Korean Culture Center of Microorganisms (KCCM) under the terms of the Budapest Treaty on Mar. 21, 2003 (Accession NO: KCCM-10476). E. coli transformed with pCK-VEGF165 (i.e., pCK vector with VEGF coding sequence—Top10-pCK/VEGF165′) was deposited at the Korean Culture Center of Microorganisms (KCCM) under the terms of the Budapest Treaty on Dec. 27, 1999 (Accession NO: KCCM-10179).

The pCK vector is constructed such that the expression of a gene, e.g., an IGF-1 gene or an HGF gene, is regulated under enhancer/promoter of the human cytomegalovirus (HCMV), as disclosed in detail in Lee et al., Biochem. Biophys. Res. Commun. 272: 230 (2000); WO 2000/040737, both of which are incorporated by reference in their entirety. pCK vector has been used for clinical trials on human body, and its safety and efficacy were confirmed (Henry et al., Gene Ther. 18:788 (2011)).

In preferred embodiments, the pCK plasmid contains a coding sequence for Class I, Ec IGF-1 isoform and/or Class I, Ea IGF-1 isoform. In particularly preferred embodiments, the pCK plasmid contains IGF-1X6 (i.e., pCK-IGF-1X6) or IGF-1X10 (i.e., pCK-IGF-1X10).

In preferred embodiments, the pCK plasmid contains a coding sequence for flHGF and/or dHGF isoforms. In particular preferred embodiments, the pCK plasmid contains HGF-X7 (i.e., pCK-HGF-X7 or VM202).

In other preferred embodiments, the plasmid is pTx (SEQ ID NO: 38), a plasmid vector derived from pCK. pTx was generated by two sequential rounds of mutagenesis of pCK. The first deletion mutagenesis was conducted to remove the unnecessary sequence between Kanamycin resistance gene and ColE1 of pCK. Specifically, deletion mutagenesis PCR was performed using a first primer pair (SEQ ID NOs: 34 and 35). The deletion of 228 base pairs between Kanamycin resistance and ColE1 was confirmed by sequencing the plasmid. The second deletion mutagenesis PCR was then performed using a second primer pair (SEQ ID NOs: 36 and 37), to optimize the size of HCMV intron sequence. HCMV intron sequence (421 base pairs) between IE1 exon 1 and exon 2 was deleted and the deletion was confirmed by sequencing.

In particular embodiments, the pTx plasmid contains IGF-1X6 (i.e., pTx-IGF-1X6) or IGF-1X10 (i.e., pTx-IGF-1X10). For example, pTx-1X10 (SEQ ID NO: 39) was generated by ligating IGF-1X10 in pTx digested with ClaI enzyme at 5′ and Sal1 enzyme at 3′.

### 6.3.3.2. Viral Vector

In other embodiments, various viral vectors known in the art can be used to deliver and express one or more IGF-1 isoforms and/or one or more HGF isoforms of the present invention. For example, vectors developed using retroviruses, lentiviruses, adenoviruses, or adeno-associated viruses can be used for some embodiments of the present invention.

(a) Retrovirus

Retroviruses capable of carrying relatively large exogenous genes have been used as viral gene delivery vectors in the senses that they integrate their genome into a host genome and have broad host spectrum.

In order to construct a retroviral vector, the polynucleotide of the invention (e.g., a coding sequence of one or more IGF-1 isoforms) is inserted into the viral genome in the place of certain viral sequences to produce a replication-defective virus. To produce virions, a packaging cell line containing the gag, pol and env genes but without the LTR (long terminal repeat) and W components is constructed (Mann et al., Cell, 33:153-159 (1983)). When a recombinant plasmid containing the polynucleotide of the invention, LTR and W is introduced into this cell line, the W sequence allows the RNA transcript of the recombinant plasmid to be packaged into viral particles, which are then secreted into the culture media (Nicolas and Rubinstein “Retroviral vectors,” In: Vectors: A survey of molecular cloning vectors and their uses, Rodriguez and Denhardt (eds.), Stoneham: Butterworth, 494-513(1988)) The media containing the recombinant retroviruses is then collected, optionally concentrated and used for gene delivery.

A successful gene transfer using the second generation retroviral vector has been reported. Kasahara et al. (Science, 266:1373-1376 (1994)) prepared variants of moloney murine leukemia virus in which the EPO (erythropoietin) sequence is inserted in the place of the envelope region, consequently, producing chimeric proteins having novel binding properties. Likely, the present gene delivery system can be constructed in accordance with the construction strategies for the second-generation retroviral vector.

(b) Lentiviruses

Lentiviruses can be also used in some embodiments of the present invention. Lentiviruses are a subclass of Retroviruses. However, Lentivirus can integrate into the genome of non-dividing cells, while Retroviruses can infect only dividing cells.

Lentiviral vectors are usually produced from packaging cell line, commonly HEK293, transformed with several plasmids. The plasmids include (1) packaging plasmids encoding the virion proteins such as capsid and the reverse transcriptase, (2) a plasmid comprising an exogenous gene (e.g., a coding sequence of one or more IGF-1 isoforms or one or more HGF isoforms) to be delivered to the target.

When the virus enters the cell, the viral genome in the form of RNA is reverse-transcribed to produce DNA, which is then inserted into the genome by the viral integrase enzyme. Thus, the exogenous delivered with the Lentiviral vector can remain in the genome and is passed on to the progeny of the cell when it divides.

(c) Adenovirus

Adenovirus has been usually employed as a gene delivery system because of its mid-sized genome, ease of manipulation, high titer, wide target-cell range, and high infectivity. Both ends of the viral genome contains 100-200 bp ITRs (inverted terminal repeats), which are cis-elements necessary for viral DNA replication and packaging. The E1 region (E1A and E1B) encodes proteins responsible for the regulation of transcription of the viral genome and a few cellular genes. The expression of the E2 region (E2A and E2B) results in the synthesis of the proteins for viral DNA replication.

Of adenoviral vectors developed so far, the replication incompetent adenovirus having the deleted E1 region is usually used. The deleted E3 region in adenoviral vectors may provide an insertion site for transgenes (Thimmappaya, B. et al., Cell, 31:543-551(1982); and Riordan, J. R. et al., Science, 245:1066-1073 (1989)). Therefore, it is preferred that the decorin-encoding nucleotide sequence is inserted into either the deleted E1 region (E1A region and/or E1B 5 region, preferably, E1B region) or the deleted E3 region. The polynucleotide of the invention may be inserted into the deleted E4 region. The term “deletion” with reference to viral genome sequences encompasses whole deletion and partial deletion as well. In nature, adenovirus can package approximately 105% of the wildtype genome, providing capacity for about 2 extra kb of DNA (Ghosh-Choudhury et al., EMBO J. 6:1733-1739 (1987)). In this regard, the foreign sequences described above inserted into adenovirus may be further inserted into adenoviral wildtype genome.

The adenovirus may be of any of the known serotypes or subgroups A-F. Adenovirus type 5 of subgroup C is the most preferred starting material for constructing the adenoviral gene delivery system of this invention. A great deal of biochemical and genetic information about adenovirus type 5 is known. The foreign genes delivered by the adenoviral gene delivery system are episomal, and genotoxicity to host cells. Therefore, gene therapy using the adenoviral gene delivery system may be considerably safe.

(d) Adeno-Associated Virus (AAV)

Adeno-associated viruses are capable of infecting non-dividing cells and various types of cells, making them useful in constructing the gene delivery system of this invention. The detailed descriptions for use and preparation of AAV vector are found in U.S. Pat. Nos. 10,308,958; 10,301,650; 10,301,648; 10,266,846; 10,265,417; 10,208,107; 10,167,454; 10,155,931; 10,149,873; 10,144,770; 10,138,295; 10,137,176; 10,113,182; 10,041,090; 9,890,365; 9,790,472; 9,770,011; 9,738,688; 9,737,618; 9,719,106; 9,677,089; 9,617,561; 9,597,363; 9,593,346; 9,587,250; 9,567,607; 9,493,788; 9,382,551; 9,359,618; 9,217,159; 9,206,238; 9,163,260; 9,133,483; 8,962,332, the disclosures of which are incorporated herein by reference in their entireties, and U.S. Pat. Nos. 5,139,941 and 4,797,368, the disclosures of which are incorporated herein by reference in their entireties.

Research results for AAV as gene delivery systems are disclosed in LaFace et al., Viology, 162: 483486 (1988), Zhou et al., Exp. Hematol. (NY), 21:928-933(1993), Walsh et al., J. Clin. Invest., 94:1440-1448(1994) and Flotte et al., Gene Therapy, 2:29-37(1995). Typically, a recombinant AAV virus is made by cotransfecting a plasmid containing the gene of interest (i.e., nucleotide sequence of interest to be delivered, e.g., a coding sequence of an IGF-1 isoform) flanked by the two AAV terminal repeats (McLaughlin et al., 1988; Samulski et al., 1989) and an expression plasmid containing the wild type AAV coding sequences without the terminal repeats (McCarty et al., J. Viral., 65:2936-2945 (1991)).

(e) Other Viral Vectors

Other viral vectors may be employed as a gene delivery system in the present invention. Vectors derived from viruses such as vaccinia virus (Puhlmann M. et al., Human Gene Therapy 10:649-657(1999); Ridgeway, “Mammalian expression vectors,” In: Vectors: A survey of molecular cloning vectors and their uses. Rodriguez and Denhardt, eds. Stoneham: Butterworth, 467-492 (1988); Baichwal and Sugden, “Vectors for gene transfer derived from animal DNA viruses: Transient and stable expression of transferred genes,” In: Kucherlapati R, ed. Gene transfer. New York: Plenum Press, 117-148 (1986) and Coupar et al., Gene, 68:1-10(1988)), lentivirus (Wang G. et al., J. Clin. Invest. 104 (11): RS 5-62 (1999)) and herpes simplex virus (Chamber R., et al., Proc. Natl. 10 15 Acad. Sci USA 92:1411-1415(1995)) may be used in the present delivery systems for transferring the polynucleotide of the invention into cells.

### 6.3.4. Administration Methods

Various methods can be used to administer the IGF-1-encoding DNA construct and the HGF-encoding DNA construct.

### 6.3.4.1.1. Injection

In some embodiments, the DNA construct is administered by injection of a liquid pharmaceutical composition. In some embodiments, the IGF-1-encoding DNA construct and the HGF-encoding DNA construct are administered together by a single injection. In some embodiments, the IGF-1-encoding DNA construct and the HGF-encoding DNA construct are administered together by multiple injections. In some embodiments, the IGF-1-encoding DNA construct and the HGF-encoding DNA construct are administered individually by multiple injections.

In currently preferred embodiments, the DNA construct is administered by intramuscular injection. Typically, the DNA construct is administered by intramuscular injection close to the site of nerve damage, site of pain or patient-perceived site of pain, or site of other symptom associated with the neuropathic disease. In some embodiments, the DNA constructs are administered to the muscles of hands, feet, legs, or arms of the subject.

In some embodiments, the construct is injected subcutaneously or intradermally. In some embodiments, the DNA construct is administered by intravascular delivery. In certain embodiments, the construct is injected by retrograde intravenous injection.

### 6.3.4.1.2. Electroporation

Transformation efficiency of a plasmid DNA into cells in vivo can in some instances be improved by performing injection followed by electroporation. Thus, in some embodiments, the DNA construct is administered by injection followed by electroporation. In particular embodiments, electroporation is administered using the TriGrid™ Delivery System (Ichor Medical Systems, Inc., San Diego, USA).

### 6.3.4.1.3. Sonoporation

In some embodiments, sonoporation is used to enhance transformation efficiency of the DNA construct of the present invention. Sonoporation utilizes ultrasound wave to temporarily permeabilize the cell membrane to allow cellular uptake of DNA. DNA constructs can be incorporated within microbubbles and administered into systemic circulation, followed by external application of ultrasound. The ultrasound induces cavitation of the microbubble within the target tissue to result in release and transfection of the constructs.

### 6.3.4.1.4. Magnetofection

In some embodiments, magnetofection is used to enhance transformation efficiency of a DNA construct of the present invention. The construct is administered after being coupled to a magnetic nanoparticle. Application of high gradient external magnets cause the complex to be captured and held at the target. The DNA construct can be released by enzymatic cleavage of cross linking molecule, charge interaction or degradation of the matrix.

### 6.3.4.1.5. Liposome

In some embodiments, DNA constructs of the present invention can be delivered by liposomes. Liposomes are formed spontaneously when phospholipids are suspended in an excess of aqueous medium. Liposome-mediated DNA delivery has been successful as described in Dos Santos Rodrigues et al., Int. J. Pharm. 566:717-730 (2019); Rasoulianboroujeni et al., Mater Sci Eng C Mater Biol Appl. 75:191-197 (2017); Xiong et al., Pharmazie 66(3):158-164 (2011); Nicolau and Sene, Biochim. Biophys. Acta, 721:185-190 (1982) and Nicolau et al., Methods Enzymol., 149:157-176 (1987). Example of commercially accessible reagents for transfecting animal cells using liposomes includes Lipofectamine (Gibco BRL). Liposomes entrapping DNA constructs of the invention interact with cells by mechanisms such as endocytosis, adsorption and fusion and then transfer the sequences into cells.

### 6.3.4.1.6. Transfection

When a viral vector is used to deliver an IGF-1-encoding DNA construct or an HGF-encoding DNA construct, the construct may be delivered into cells by various viral infection methods known in the art. Infection of host cells using viral vectors is known in the art.

The pharmaceutical composition of this invention can be administered parenterally. For non-oral administration, intravenous injection, intraperitoneal injection, intramuscular injection, subcutaneous injection, or local injection can be employed. For example, the pharmaceutical composition can be injected by retrograde intravenous injection.

Preferably, the pharmaceutical composition of the present invention can be administered into the muscle. In some embodiments, the administration is targeted to the muscle affected by neuropathy (e.g., neuropathic pain or other symptoms).

### 6.3.5. Dose

The IGF-1-encoding DNA construct and the HGF-encoding DNA construct are administered in therapeutically effective amounts. In the methods described herein, the therapeutically effective amount, or dose, of a DNA construct is a dose effective to treat neuropathy in the subject by itself, in combination with a different DNA construct, or in combination with other therapeutic agent.

In some embodiments of the methods described herein, each of the DNA constructs (IGF-1-encoding DNA construct and HGF-encoding DNA construct) is administered at a total dose of 1 μg to 200 mg, 1 mg to 200 mg, 1 mg to 100 mg, 1 mg to 50 mg, 1 mg to 20 mg, 2 mg to 10 mg, 16 mg, 8 mg, 4 mg or 2 mg.

In some embodiments, the total dose of an IGF-1-encoding DNA construct and the total dose of an HGF-encoding DNA construct that are administered to a subject are same. In some embodiments, the total dose of an IGF-1-encoding DNA construct and the total dose of an HGF-encoding DNA construct are different. In some embodiments, the total dose of an IGF-1-encoding DNA construct is adjusted depending on the total dose of an HGF-encoding DNA construct. In some embodiments, the total dose of an HGF-encoding DNA construct is adjusted depending on the total dose of an IGF-1-encoding DNA construct.

In typical embodiments, the total dose of each DNA construct is divided into a plurality of individual injection doses. In some embodiments, the total dose is divided into a plurality of equal injection doses. In some embodiments, the total dose is divided into unequal injection doses.

In various divided dose embodiments, the total dose of each DNA construct is administered to 4, 8, 16, 24, 32 or 64 different injection sites.

In some embodiments, the dose of each DNA construct per injection is between 0.1 and 20 mg, between 1 and 10 mg, between 2 and 8 mg, or between 3 and 8 mg. In certain embodiments, the dose of each DNA construct per injection is 0.1 mg, 0.15 mg, 0.2 mg, 0.25 mg, 0.3 mg, 0.35 mg, 0.4 mg, 0.45 mg, 0.5 mg, 1 mg, 2 mg, 4 mg, 8 mg, 16 mg, or 32 mg.

In some embodiments, IGF-1-encoding DNA construct and HGF-encoding DNA construct are administered together. In the cases, the dose of two DNA constructs in combination is between 0.1 and 20 mg, between 1 and 10 mg, between 2 and 8 mg, or between 3 and 8 mg per injection. In certain embodiments, the dose of two DNA constructs in combination is 0.1 mg, 0.15 mg, 0.2 mg, 0.25 mg, 0.3 mg, 0.35 mg, 0.4 mg, 0.45 mg, 0.5 mg, 1 mg, 2 mg, 4 mg, 8 mg, 16 mg, or 32 mg per injection.

The total dose of each DNA construct, or both DNA constructs in combination can be administered during one visit or over two or more visits.

In typical divided dose embodiments, all of the plurality of injection doses are administered within 1 hour of one another. In some embodiments, all of the plurality of injection doses are administered within 1.5, 2, 2.5 or 3 hours of one another.

In various embodiments of the methods, a total dose of each DNA construct or a total dose of two DNA constructs in combination, whether administered as a single unitary dose or divided into plurality of injection doses, is administered only once to the subject.

In some embodiments, administration of a total dose of each DNA construct or two DNA constructs in combination into a plurality of injection sites over one, two, three or four visits can comprise a single cycle. For example, administration of 64 mg, 32 mg, 16 mg, 8 mg, 4 mg or 2 mg of each DNA construct into a plurality of injection sites over two visits can comprise a single cycle. The two visits can be 3, 5, 7, 14, 21 or 28 days apart.

In some embodiments, administration of an IGF-1-encoding DNA construct and administration of an HGF-encoding DNA construct into a plurality of injection sites over one, two, three or four visits can comprise a single cycle. For example, administration of 64 mg, 32 mg, 16 mg, 8 mg, 4 mg or 2 mg of IGF-1-encoding DNA construct into a plurality of injection sites and administration of 64 mg, 32 mg, 16 mg, 8 mg, 4 mg or 2 mg of HGF-encoding DNA construct into a plurality of injection sites over two visits can comprise a single cycle. The two visits can be 3, 5, 7, 14, 21 or 28 days apart.

In some embodiments, the cycle can be repeated. The cycle can be repeated twice, three times, four times, five times, six times, or more.

In some embodiments, the cycle can be repeated 1, 2, 3, 4, 5, 6, 7, 8, 9, 10, 11, 12, or more months after the previous cycle.

In some embodiments, the total dose administered in the subsequent cycle is same as the total dose administered in the prior cycle. In some embodiments, the total dose administered in the subsequent cycle is different from the total dose administered in the prior cycle.

In preferred embodiments, the DNA construct (IGF-1-encoding DNA construct or HGF-encoding DNA construct) is administered at a dose of 8 mg per affected limb, equally divided into a plurality of intramuscular injections and plurality of visits, wherein each of the plurality of injections in any single visit is performed at a separate injection site. In certain embodiments, the DNA construct (IGF-1-encoding DNA construct or HGF-encoding DNA construct) is administered at a dose of 8 mg per affected limb, equally divided into a first dose of 4 mg per limb on day 0 and a second dose of 4 mg per limb on day 14, wherein each of the first and second dose is equally divided into a plurality of injection doses.

In some embodiments, IGF-1-encoding DNA construct and HGF-encoding DNA construct is administered concurrently or separately at a total dose of 16 mg per affected limb, equally divided into a plurality of intramuscular injection and plurality of visits, wherein each of the plurality of injections in any single visit is performed at a separate injection site. In some embodiments, the administration of IGF-1-encoding DNA construct at a dose of 8 mg per affected limb and the administration of HGF-encoding DNA construct at a dose of 8 mg per affected limb constitutes one cycle. The cycle can be repeated once, twice, three times or more.

The actual amount administered, and rate and time-course of administration, will depend on the nature and severity of neuropathy being treated. In typical embodiments, one or more DNA constructs are administered in an amount effective to reduce symptoms of neuropathy, for example, neuropathic pain. In some embodiments, the amount is effective to reduce the symptom of neuropathy within 1 week of administration. In some embodiments, the amount is effective to reduce the symptom of neuropathy within 2 weeks, 3 weeks, or 4 weeks of administration.

In some embodiments, two different types of IGF-1-encoding DNA constructs or two different types of HGF-encoding DNA constructs are administered together. In some embodiments, a dual expression construct is delivered to induce expression of two isoforms of IGF-1 or HGF.

According to the conventional techniques known to those skilled in the art, the pharmaceutical composition may be formulated with pharmaceutically acceptable carrier and/or vehicle as described above, finally providing several forms a unit dose form and a multi-dose form. Non-limiting examples of the formulations include, but not limited to, a solution, a suspension or an emulsion in oil or aqueous medium, an extract, an elixir, a powder, a granule, a tablet and a capsule, and may further comprise a dispersion agent or a stabilizer.

In vivo and/or in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

The DNA constructs can be administered by themselves or in combination with other treatments, either simultaneously or sequentially.

### 6.3.6. Patients with Neuropathy

In the methods described herein, the patients selected for treatment have neuropathy. The patients can have peripheral neuropathy, cranial neuropathy, autonomic neuropathy or focal neuropathy. The neuropathy can be caused by diseases, injuries, infections or vitamin deficiency states. For example, the neuropathy can be caused by diabetes, vitamin deficiencies, autoimmune diseases, genetic or inherited disorders, amyloidosis, uremia, toxins or poisons, trauma or injury, tumors, or can be idiopathic. In some embodiments, the patients have diabetic peripheral neuropathy.

The patients can have one or more symptoms associated with neuropathy, such as pain (neuropathic pain), other sensory defects (e.g., loss of feeling, numbness, tingling, etc.), motor defects (e.g., weakness, loss of reflexes, loss of muscle mass, cramping, loss of dexterity, etc.), and autonomic dysfunction (e.g., nausea, vomiting, impotence, dizziness, constipation, diarrhea, etc.).

The patients can be treated by one or more treatment methods known in the art in addition to the treatment method provided herein.

Treatment methods of the present invention can be used to treat a human patient or an animal with neuropathy.

### 6.3.7. Order of Administration

The methods described herein comprise the steps of administering a therapeutically effective amount of a first IGF-1-encoding DNA construct capable of expressing a human IGF-1 isoform and administering a therapeutically effective amount of a first HGF-encoding DNA construct capable of expressing a human HGF isoform. The therapeutically effective amount is an amount effective in treating the disease in combination or individually.

The step of administering a first IGF-1-encoding DNA construct and the step of administering a first HGF-encoding DNA construct can be performed concurrently or sequentially. In some embodiments, administration of a first IGF-1-encoding DNA construct and administration of a first HGF-encoding DNA construct is performed separately, at least a few minutes apart, a few hours apart, one day apart, two days apart, three days apart, one week apart, two weeks apart, three weeks apart, one month apart, two month apart, three months apart, or six months apart. In some embodiments, the step of administering a first HGF-encoding DNA construct is performed before the step of administering a first IGF-1-encoding DNA construct. In some embodiments, the step of administering a first IGF-1-encoding DNA construct is performed before the step of administering a first HGF-encoding DNA construct.

In some embodiments, the step of administering a first IGF-1-encoding DNA construct, the step of administering a first HGF-encoding DNA construct, or both are repeated. In some embodiments, the step is repeated twice, three times, or more.

The first IGF-1-encoding DNA construct can be any of the IGF-1-encoding DNA constructs provided herein or a modification thereof. It can express one or more IGF-1 isoforms. It can be a DNA construct encoding one IGF-1 isoform, Class I, Ec (SEQ ID NO: 16); Class II, Ea (SEQ ID NO: 18); Class I, Eb (SEQ ID NO: 20); or Class I, Ea isoforms (SEQ ID NO: 14). In can be a dual expression DNA construct encoding two IGF-1 isoforms. In some embodiments, the DNA construct can encode Class I, Ec (SEQ ID NO: 16) and Class I, Ea isoforms (SEQ ID NO: 14).

The first HGF-encoding DNA construct can be any of the HGF-encoding DNA constructs provided herein or a modification thereof. It can express one or more HGF isoforms. It can be a DNA construct encoding one HGF isoform, flHGF (SEQ ID NO: 11) or dHGF (SEQ ID NO: 12). It can be a dual expression DNA construct encoding two HGF isoforms. In preferred embodiments, the DNA construct comprises a polynucleotide of SEQ ID NO: 13. It can be VM202.

The method can further comprise the step of administering a second IGF-1-encoding DNA construct. The second IGF-1-encoding DNA construct can be same as or different from the first IGF-1-encoding DNA construct. The second IGF-1-encoding DNA construct can be any of the IGF-1-encoding DNA construct provided herein or a modification thereof. The step of administering a first IGF-1-encoding DNA construct and the step of administering a second IGF-1-encoding DNA construct can be performed concurrently or sequentially. In some embodiments, a first IGF-1-encoding DNA construct capable of expressing Class I, Ec (SEQ ID NO: 16) and a second IGF-1-encoding DNA construct capable of expressing Class I, Ea isoforms (SEQ ID NO: 14) are administered concurrently. In some embodiments, administration of a first IGF-1-encoding DNA construct and administration of a second IGF-1-encoding DNA construct are performed separately, at least a few minutes apart, a few hours apart, one day apart, two days apart, three days apart, one week apart, two weeks apart, three weeks apart, one month apart, two month apart, three months apart, or six months apart.

The method can further comprise the step of administering a second HGF-encoding DNA construct. The second HGF-encoding DNA construct can be same as or different from the first HGF-encoding DNA construct. The second HGF-encoding DNA construct can be any of the HGF-encoding DNA construct provided herein or a modification thereof. The step of administering a first HGF-encoding DNA construct and the step of administering a second HGF-encoding DNA construct can be performed concurrently or sequentially. For example, a first HGF-encoding DNA construct capable of expressing flHGF (SEQ ID NO: 11) and a second HGF-encoding DNA construct capable of expressing dHGF (SEQ ID NO: 12) can be administered concurrently. In some embodiments, administration of a first HGF-encoding DNA construct and administration of a second HGF-encoding DNA construct is performed separately, at least a few minutes apart, a few hours apart, one day apart, two days apart, three days apart, one week apart, two weeks apart, three weeks apart, one month apart, two month apart, three months apart, or six months apart.

In some embodiments, the method comprises administration of VM202 together with pCK-IGF-1X6 or pCK-IGF-1X10. In some embodiments, the method comprises administration of other HGF-encoding DNA construct (e.g., a construct comprising a polynucleotide of SEQ ID NO: 33) together with pCK-IGF-1X6 or pCK-IGF-1X10.

In some embodiments, the method comprises administration of VM202 together with pTx-IGF-1X6 or pTx-IGF-1X10. In some embodiments, the method comprises administration of other HGF-encoding DNA construct (e.g., a construct comprising a polynucleotide of SEQ ID NO: 33) together with pTx-IGF-1X6 or pTx-IGF-1X10.

In some embodiments, the method comprises administration of VM202 followed by administration of pCK-IGF-1X6 or pCK-IGF-1X10. In some embodiments, the method comprises administration of other HGF-encoding DNA construct (e.g., pCK-HGF₇₂₈ which is a construct comprising a polynucleotide of SEQ ID NO: 33) followed by administration of pCK-IGF-1X6 or pCK-IGF-1X10. In some embodiments, the method comprises administration of pCK-IGF-1X6 or pCK-IGF-1X10 followed by administration of VM202 or other HGF-encoding DNA construct (e.g., pCK-HGF₇₂₈).

In some embodiments, the method comprises administration of VM202 followed by administration of pTx-IGF-1X6 or pTx-IGF-1X10. In some embodiments, the method comprises administration of other HGF-encoding DNA construct (e.g., pCK-HGF₇₂₈ which is a construct comprising a polynucleotide of SEQ ID NO: 33) followed by administration of pTx-IGF-1X6 or pTx-IGF-1X10. In some embodiments, the method comprises administration of pTx-IGF-1X6 or pTx-IGF-1X10 followed by administration of VM202 or other HGF-encoding DNA construct (e.g., pCK-HGF₇₂₈).

### 6.4. Pharmaceutical Composition Comprising IGF-1-Encoding DNA Construct and HGF-1 Encoding DNA Construct

In another aspect, a pharmaceutical composition comprising an IGF-1-encoding DNA construct and an HGF-encoding DNA construct is provided.

### 6.4.1. Pharmaceutical Compositions and Unit Dosage Forms Adapted for Injection

For intravenous, intramuscular, intradermal, or subcutaneous injection, the DNA constructs can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives, as required.

In some embodiments, the pharmaceutical composition comprises a DNA construct encoding one IGF-1 isoform. For example, the DNA construct can express Class I, Ec isoform (Isoform #1); Class II, Ea isoform (Isoform #2); Class I, Eb isoform (Isoform #3); or Class I, Ea isoform (Isoform #4). The DNA constructs can be pCK-IGF-1 #1, pCK-IGF-1 #2, pCK-IGF-1 #3, or pCK-IGF-1 #4. In some embodiments, the DNA constructs can be pTx-IGF-1 #1, pTx-IGF-1 #2, pTx-IGF-1 #3, or pTx-IGF-1 #4.

In some embodiments, the pharmaceutical composition comprises more than one DNA construct, each encoding one IGF-1 isoform. For example, the pharmaceutical composition can comprise (i) a first DNA construct encoding Class I, Ec isoform (Isoform #1) and a second DNA construct encoding Class II, Ea isoform (Isoform #2); (ii) a first DNA construct encoding Class I, Ec isoform (Isoform #1) and a second DNA construct encoding Class I, Eb isoform (Isoform #3); (iii) a first DNA construct encoding Class I, Ec isoform (Isoform #1) and a second DNA construct encoding Class I, Ea isoform (Isoform #4); (iv) a first DNA construct encoding Class II, Ea isoform (Isoform #2) and a second DNA construct encoding Class I, Eb isoform (Isoform #3); (v) a first DNA construct encoding Class II, Ea isoform (Isoform #2) and a second DNA construct encoding Class I, Ea isoform (Isoform #4); (vi) a first DNA construct encoding Class I, Eb isoform (Isoform #3) and a second DNA construct encoding Class I, Ea isoform (Isoform #4).

In some embodiments, the pharmaceutical composition comprises a dual expression construct, a DNA construct that can express more than one IGF-1 isoforms. For example, the pharmaceutical composition can comprise a dual expression construct that can express (i) Class I, Ec isoform (Isoform #1) and Class II, Ea isoform (Isoform #2); (ii) Class I, Ec isoform (Isoform #1) and Class I, Eb isoform (Isoform #3); (iii) Class I, Ec isoform (Isoform #1) and Class I, Ea isoform (Isoform #4); (iv) Class II, Ea isoform (Isoform #2) and Class I, Eb isoform (Isoform #3); (v) Class II, Ea isoform (Isoform #2) and Class I, Ea isoform (Isoform #4); (vi) Class I, Eb isoform (Isoform #3) and Class I, Ea isoform (Isoform #4).

In some embodiments, the pharmaceutical composition comprises a dual expression construct, pCK-IGF-1X6 or pCK-IGF-1X10. In some embodiments, the pharmaceutical composition comprises a dual expression construct, pTx-IGF-1X6 or pTx-IGF-1X10. In some embodiments, the pharmaceutical composition comprises two dual expression constructs, for example, including both pCK-IGF-1X6 and pCK-IGF-1X10. In some embodiments, the pharmaceutical composition comprises two dual expression constructs, for example, including both pTx-IGF-1X6 and pTx-IGF-1X10.

In some embodiments, the pharmaceutical composition further comprises a DNA construct encoding one HGF isoform. For example, the DNA construct can express flHGF or dHGF. In some embodiments, the pharmaceutical composition comprises more than one DNA constructs, each encoding one HGF isoform. For example, the pharmaceutical composition can comprise a first DNA construct encoding flHGF and a second DNA construct encoding dHGF.

In some embodiments, the pharmaceutical composition comprises a dual expression construct, a DNA construct that can express more than one HGF isoforms. For example, the pharmaceutical composition can comprise a dual expression construct that can express both flHGF and dHGF.

In preferred embodiments, the pharmaceutical composition comprises a dual expression construct, pCK-HGF-X7 (VM202). In some embodiments, the pharmaceutical composition comprises two HGF-encoding DNA constructs, each encoding flHGF or dHGF. In some embodiments, the pharmaceutical composition comprise one HGF-encoding DNA construct, capable of expressing flHGF (pCK-HGF₇₂₈).

In some embodiments, the pharmaceutical composition further comprises another therapeutic agent. For example, the pharmaceutical composition can further comprise another therapeutic agent effective in treating neuropathy.

In various embodiments, one or more DNA constructs are present in the liquid composition at a concentration of 0.01 mg/ml, 0.05 mg/ml, 0.1 mg/ml, 0.25 mg/ml, 0.45 mg/ml, 0.5 mg/ml, or 1 mg/ml, individually or in combination. In some embodiments, the unit dosage form is a vial containing 2 ml of the pharmaceutical composition with one or more DNA constructs at a concentration of 0.01 mg/ml, 0.1 mg/ml, 0.5 mg/ml, or 1 mg/ml, individually or in combination. In some embodiments, the unit dosage form is a vial containing 1 ml of the pharmaceutical composition with one or more DNA constructs at a concentration of 0.01 mg/ml, 0.1 mg/ml, 0.5 mg/ml, or 1 mg/ml, individually or in combination. In some embodiments, the unit dosage form is a vial containing less than 1 ml of the pharmaceutical composition with one or more DNA constructs at a concentration of 0.01 mg/ml, 0.1 mg/ml, 0.5 mg/ml, or 1 mg/ml, individually or in combination.

In some embodiments, the unit dosage form is a vial, ampule, bottle, or pre-filled syringe. In some embodiments, the unit dosage form contains 0.01 mg, 0.1 mg, 0.2 mg, 0.25 mg, 0.5 mg, 1 mg, 2.5 mg, 5 mg, 8 mg, 10 mg, 12.5 mg, 16 mg, 24 mg, 25 mg, 50 mg, 75 mg, 100 mg, 150 mg, or 200 mg of one or more DNA constructs of the present invention.

In some embodiments, the pharmaceutical composition in the unit dosage form is in liquid form. In various embodiments, the unit dosage form contains between 0.1 ml and 50 ml of the pharmaceutical composition. In some embodiments, the unit dosage form contains 0.25 ml, 0.5 ml, 1 ml, 2.5 ml, 5 ml, 7.5 ml, 10 ml, 25 ml, or 50 ml of pharmaceutical composition.

In particular embodiments, the unit dosage form is a vial containing 0.5 ml, 1 ml, 1.5 ml or 2 ml of the pharmaceutical composition at unit dosage form embodiments suitable for subcutaneous, intradermal, or intramuscular administration include preloaded syringes, auto-injectors, and auto-inject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single-use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an auto-inject pen. The auto-inject pen comprises an auto-inject pen containing a pharmaceutical composition as described herein. In some embodiments, the auto-inject pen delivers a predetermined volume of pharmaceutical composition. In other embodiments, the auto-inject pen is configured to deliver a volume of pharmaceutical composition set by the user.

In various embodiments, the auto-inject pen contains about 0.1 mL to about 5.0 mL of the pharmaceutical composition. In specific embodiments, the auto-inject pen contains about 0.5 mL of the pharmaceutical composition. In particular embodiments, the auto-inject pen contains about 1.0 mL of the pharmaceutical composition. In other embodiments, the auto-inject pen contains about 5.0 mL of the pharmaceutical composition.

### 6.4.2. Lyophilized DNA Formulations

In some embodiments, DNA constructs of the present inventions are formulated as a lyophilized composition. In specific embodiments, DNA constructs are lyophilized as disclosed in U.S. Pat. No. 8,389,492, incorporated by reference in its entirety herein.

In some embodiments, DNA constructs are formulated with certain excipients, e.g., a carbohydrate and a salt, prior to lyophilization. Stability of the DNA construct to be utilized as a diagnostic or therapeutic agent can be increased by formulating the DNA construct prior to lyophilization with an aqueous solution comprising a stabilizing amount of carbohydrate.

The carbohydrate can be a mono-, oligo-, or polysaccharide, such as sucrose, glucose, lactose, trehalose, arabinose, pentose, ribose, xylose, galactose, hexose, idose, mannose, talose, heptose, fructose, gluconic acid, sorbitol, mannitol, methyl a-glucopyranoside, maltose, isoascorbic acid, ascorbic acid, lactone, sorbose, glucaric acid, erythrose, threose, allose, altrose, gulose, erythrulose, ribulose, xylulose, psicose, tagatose, glucuronic acid, galacturonic acid, mannuronic acid, glucosamine, galactosamine, neuraminic acid, arabinans, fructans, fucans, galactans, galacturonans, glucans, mannans, xylans, levan, fucoidan, carrageenan, galactocarolose, pectins, pectic acids, amylose, pullulan, glycogen, amylopectin, cellulose, dextran, cyclodextrin, pustulan, chitin, agarose, keratin, chondroitin, dermatan, hyaluronic acid, alginic acid, xantham gum, or starch.

In one series of embodiments, the carbohydrate is mannitol or sucrose.

The carbohydrate solution prior to lyophilization can correspond to carbohydrate in water alone, or a buffer can be included. Examples of such buffers include PBS, HEPES, TRIS or TRIS/EDTA. Typically the carbohydrate solution is combined with the DNA construct to a final concentration of about 0.05% to about 30% sucrose, typically 0.1% to about 15% sucrose, such as 0.2% to about 5%, 10% or 15% sucrose, preferably between about 0.5% to 10% sucrose, 1% to 5% sucrose, 1% to 3% sucrose, and most preferably about 1.1% sucrose.

DNA formulation of the invention can also include a salt, e.g., NaCl or KCl. In some embodiments, the salt is NaCl. In some embodiments, the salt of the DNA formulation is in an amount selected from the group consisting of between about 0.001% to about 10%, between about 0.1% and 5%, between about 0.1% and 4%, between about 0.5% and 2%, between about 0.8% and 1.5%, between about 0.8% and 1.2% w/v. In certain embodiments, the salt of the DNA formulation is in an amount of about 0.9% w/v.

The final concentration of one or more DNA constructs in liquid compositions reconstituted from lyophilized formulations can be from about 1 ng/mL to about 30 mg/mL. For example, the final concentration can be about 1 ng/mL, about 5 ng/mL, about 10 ng/mL, about 50 ng/mL, about 100 ng/mL, about 200 ng/mL, about 500 ng/mL, about 1 μg/mL, about 5 g/mL, about 10 μg/mL, about 50 μg/mL, about 100 μg/mL, about 200 μg/mL, about 400 g/mL, about 500 μg/mL, about 600 μg/mL, about 800 μg/mL, about 1 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 7 mg/mL, about 8 mg/mL, about 9 mg/mL, about 10 mg/mL, about 20 mg/mL, or about 30 mg mg/mL, individually or in combination. In certain embodiments of the invention, the final concentration of one or more DNA constructs is from about 100 μg/mL to about 2.5 mg/mL individually or in combination. In particular embodiments of the invention, the final concentration of one or more DNA construct is from about 0.5 mg/mL to 1 mg/mL, individually or in combination.

The DNA formulation of the invention is lyophilized under standard conditions known in the art. A method for lyophilization of the DNA formulation of the invention may comprise (a) loading a container (e.g., a vial), with a DNA formulation (e.g., a DNA formulation comprising one or more DNA constructs of the present invention), a salt and a carbohydrate, into a lyophilizer, wherein the lyophilizer has a starting temperature of about 5° C. to about −50° C.; (b) cooling the DNA formulation to subzero temperatures (e.g., −10° C. to −50° C.); and (c) substantially drying the DNA formulation. The conditions for lyophilization, e.g., temperature and duration, of the DNA formulation of the invention can be adjusted by a person of ordinary skill in the art taking into consideration factors that affect lyophilization parameters, e.g., the type of lyophilization machine used, the amount of DNA used, and the size of the container used.

The container holding the lyophilized DNA formulation may then be sealed and stored for an extended period of time at various temperatures (e.g., room temperature to about −180° C., preferably about 2-8° C. to about −80° C., more preferably about −20° C. to about −80° C., and most preferably about −20° C.). In certain aspects, the lyophilized DNA formulations are preferably stable within a range of from about 2-8° C. to about −80° C. for a period of at least 6 months without losing significant activity. Stable storage plasmid DNA formulation can also correspond to storage of plasmid DNA in a stable form for long periods of time before use as such for research or plasmid-based therapy. Storage time may be as long as several months, 1 year, 5 years, 10 years, 15 years, or up to 20 years. Preferably the preparation is stable for a period of at least about 3 years.

### 6.5. Kits for Combination Therapy

In another aspect, the present invention provides a kit for a combination therapy with an IGF-1-encoding DNA construct and an HGF-encoding DNA construct.

The kit can comprise a first pharmaceutical composition comprising an IGF-1-encoding DNA construct and a second pharmaceutical composition comprising an HGF-encoding DNA construct. In some embodiments, the first pharmaceutical composition and the second pharmaceutical composition are the same pharmaceutical composition in a single container. In some embodiments, the first pharmaceutical composition and the second pharmaceutical composition are separate pharmaceutical compositions in two or more separate containers.

The first pharmaceutical composition can comprise any of the IGF-1-encoding DNA construct provided herein. For example, the IGF-1-encoding DNA construct can be a single expressing DNA construct capable of expressing one IGF-1 isoform, or a dual expression DNA construct expressing two IGF-1 isoforms. The second pharmaceutical composition can comprise any of the HGF-encoding DNA construct provided herein. For example, the HGF-encoding DNA construct can be a single expressing DNA construct capable of expressing one HGF isoform, or a dual expression DNA construct expressing two HGF isoforms.

The kit can comprise one or more unit doses of IGF-1-encoding DNA construct, HGF-encoding DNA construct or both.

The kit can further comprise an instruction explaining the method of administering the IGF-1-encoding DNA construct, HGF-encoding DNA construct or both. The method can be any of the administration methods provided herein.

### 6.6. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art.

### 6.6.1. Example 1: Therapeutic Efficacy of Co-Administration of IGF-1-Encoding DNA Constructs and HGF-Encoding DNA Construct in a Murine CCI Neuropathy Model

Therapeutic effects of co-administration of an IGF-1-encoding construct and an HGF-encoding construct were tested in the chronic constriction injury (CCI) model in mice, which is a widely accepted model for studying neuropathy. The CCI mouse model was generated by the chronic constriction injury (CCI) of the sciatic nerve, which is known to initiate several processes that result in chronic nerve injury in the periphery. The CCI mice are also known to have neuropathic pain.

Specifically, 4-week old male ICR mice (with between 24 and 26 g of body weight) were purchased, and used for generating the CCI model and testing the effects. All surgical and experimental procedures were approved by the Institutional Animal Care and Use committee at Seoul National University. In order to perform CCI in mice, they were given about 1-cm long blunt dissections to expose the right sciatic nerve, which usually lies between the gluteus and biceps femoris muscles. Once the sciatic nerve proximal to the trifurcation site was exposed, it was given loose ligatures three times with 0.5 mm spacing using 6-0 silk (Ethicon) sutures. The ligatures were slightly tightened until there was a noticeable twitch of the right hind limb. Sham-operated mice were given the same dissections in the right thigh, but did not receive ligatures in the sciatic nerve.

Total 200 μg of DNA constructs were intramuscularly injected on the day of CCI, and the pain sensitivity towards mechanical stimuli was measured by Von Frey's filament. Types and amounts of DNA constructs injected in each group is summarized below in TABLE 1.

TABLE 1

| | IGF-1-encoding | HGF-encoding | Other DNA |
|---|---|---|---|
| Group ID | DNA construct | DNA construct | construct |
| pCK | None | None | 200 μg |
| | | | pCK |
| VM202 | | 200 μg VM202 | None |
| VM202 + | 100 μg pCK-IGF-1#1 | 100 μg VM202 | None |
| IGF-1#1 | | | |
| VM202 + | 100 μg pCK-IGF-1#2 | 100 μg VM202 | None |
| IGF-1#2 | | | |
| VM202 + | 100 μg pCK-IGF-1#3 | 100 μg VM202 | None |
| IGF-1#3 | | | |
| VM202 + | 100 μg pCK-IGF-1#4 | 100 μg VM202 | None |
| IGF-1#4 | | | |
| VM202 + | 50 μg pCK-IGF-1#1 + | 100 μg VM202 | None |
| IGF-1#1 + 4 | 50 μg pCK-IGF-1#4 | | |

Each group consisted of 6 mice, and more than 2 independent experiments were performed (mean ± SEM; *, p < 0.05; **, p < 0.01; ***, p < 0.001).

In TABLE 1, pCK-IGF-1 #1, pCK-IGF-1 #2, pCK-IGF-1 #3, and pCK-IGF-1 #4 are DNA constructs encoding an individual human IGF-1 isoform cloned in the pCK vector. The DNA constructs were constructed in the pCK vector using standard molecular cloning techniques. Specifically, four polynucleotides (SEQ ID NO: 15, 17, 19 and 21) were obtained by customized DNA synthesis process provided by Bioneer (Korea). These polynucleotides were synthesized with 5′ linker, Cla I and 3′ linker, Sal I. pCK vector and the polynucleotides were restricted with Cla I and Sal I. IGF-1 #1 encoding Class I, Ec (Isoform #1) was generated by inserting a polynucleotide of SEQ ID NO: 17, which is a coding sequence of Class I, Ec isoform and comprises at least a part of exons 1, 3/4, 5 and 6 of IGF-1 gene, into the cloning site in pCK vector. IGF-1 #2 encoding Class II, Ea (Isoform #2) was generated by inserting a polynucleotide of SEQ ID NO: 19, which is a coding sequence of Class II, Ea isoform and comprises at least a part of exons 2, 3/4 and 6 of IGF-1 gene, into the cloning site in pCK vector. IGF-1 #3 encoding Class I, Eb (Isoform #3) was generated by inserting a polynucleotide of SEQ ID NO: 21, which is a coding sequence of Class I, Eb isoform and comprises at least a part of exons 1, 3/4 and 5 of IGF-1 gene, into the cloning site in pCK vector. IGF-1 #4 encoding Class I, Ea (Isoform #4) was generated by inserting a polynucleotide of SEQ ID NO:15 and comprises at least a part of exons 1, 3/4 and 6, into the cloning site in pCK vector. Expression of each IGF-1 isoform from each plasmid was tested and confirmed both in vitro and in vivo.

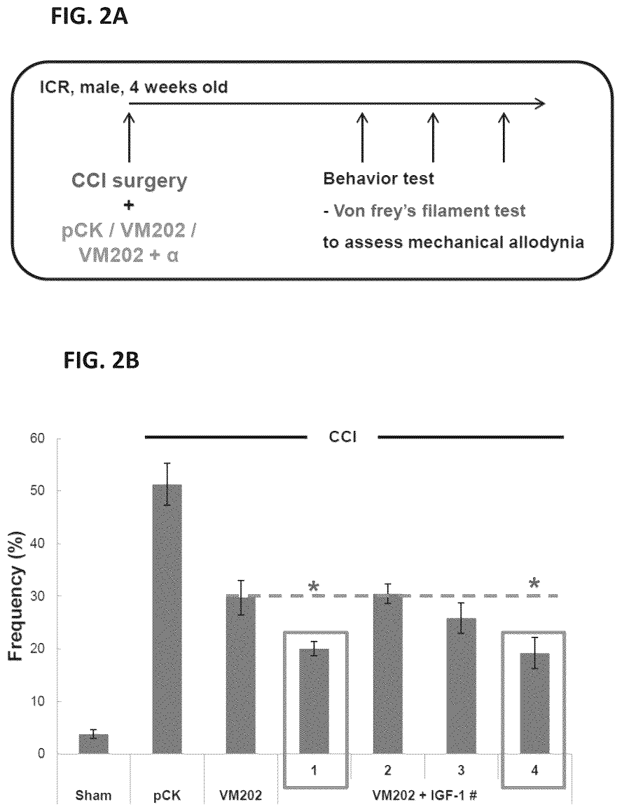

One week after CCI surgery and administration of the DNA constructs, the development of mechanical allodynia was assessed using Von Frey's filament, and pain symptoms were evaluated weekly as provided in FIG. 2A. Von Frey's filament test was performed to measure the mechanical sensitivity of mice. Briefly, animals were placed individually in the cylinder on top of the metal mesh floor for adaptation. The frequency of mechanical sensitivity of mice was assessed by stimulating the hind paw using constant thickness of the filament (0.16 g).

FIG. 2B is a graph summarizing the frequency (%) of paw withdrawal measured in the CCI experiment described in FIG. 2A. The frequency (%) of FIG. 2B is an average of measurements taken at 1 week to 4 weeks following CCI surgery. The result demonstrates that injection of VM202 alone, or VM202 in combination of various IGF-1-encoding DNA constructs, provides significant reduction in paw withdrawal frequency as compared to vector alone (pCK). Furthermore, the injection of VM202 in combination of pCK-IGF-1 #1 or pCK-IGF-1 #4, (i.e., a DNA construct capable of expressing IGF-1 isoform #1 or IGF-1 isoform #4) provided more significant reduction than injection of VM202 alone, or VM202 in combination with IGF-1 #2 or IGF-1 #3. The data suggest that IGF isoform #1 (Class I, Ec) and IGF isoform #4 (Class I, Ea) are particularly effective in treating neuropathy when administered together with VM202.

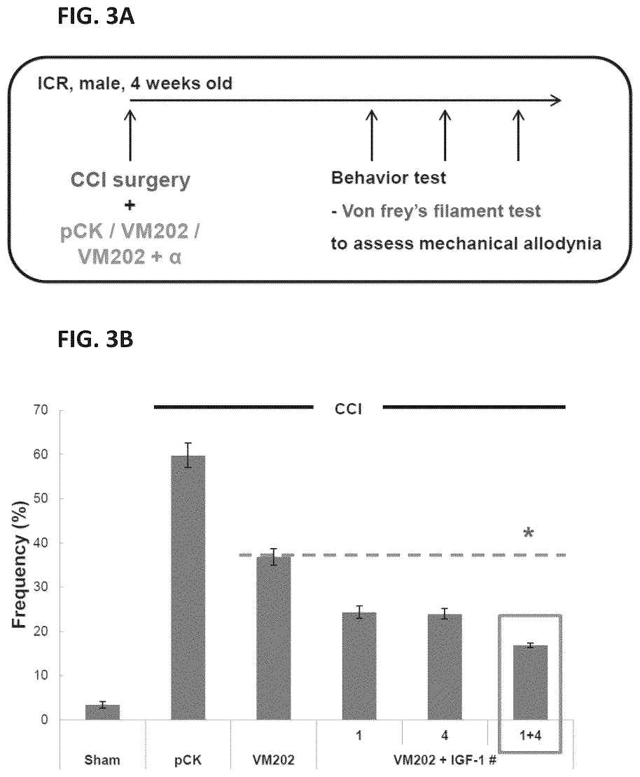

IGF-1 #1 and IGF-1 #4, found most effective in the data provided in FIG. 2B, were further tested to see whether their effects can be enhanced when administered together. Specifically, 50 μg of IGF-1 #1 and 50 μg of IGF-1 #4 were administered to CCI mice together with VM202 and their paw withdrawal frequency was measured as summarized in FIG. 3A. The result (average of 1 week to 4 weeks) provided in FIG. 3B demonstrates that injection of VM202 in combination with both pCK-IGF-1 #1 and pCK-IGF-1 #4 provided even more significant reduction in paw withdrawal frequency compared to VM202 in combination with pCK-IGF-1 #1 alone, or VM202 in combination with pCK-IGF-1 #4 alone. The data suggest that IGF isoform #1 (Class I, Ec) and IGF isoform #4 (Class I, Ea) in combination have even greater therapeutic efficacy when administered together with VM202.

### 6.6.2. Example 2: Therapeutic Efficacy of Serial Administration of IGF-1-Encoding DNA Constructs and HGF-Encoding DNA Construct in a Murine CCI Neuropathy Model

CCI neuropathy mice were generated as provided in Example 1 and divided into seven groups as provided in TABLE 2. Total 200 μg of DNA constructs were intramuscularly injected into the CCI mice on the day of CCI surgery (1^{st} injection), and another injection was performed at week 3 (2^{nd} injection). DNA constructs administered in the 1^{st} injection and the 2^{nd} injection for each group are summarized below in TABLE 2. Each group consisted of 6 mice, and more than 2 independent experiments were performed (mean±SEM; *, p<0.05;**, p<0.01; ***, p<0.001).

TABLE 2

| Group ID | 1^{st} injection (week 0) | 2^{nd} injection (week 3) |
|---|---|---|
| pCK | 100 μg pCK | 100 μg pCK |
| IGF-1 -> IGF-1 | 50 μg pCK-IGF-1#1 + | 50 μg pCK-IGF-1#1 + |
| | 50 μg pCK-IGF-1#4 | 50 μg pCK-IGF-1#4 |
| VM202 -> pCK | 100 μg VM202 | 100 μg pCK |
| IGF-1 -> pCK | 50 μg pCK-IGF-1#1 + | 100 μg pCK |
| | 50 μg pCK-IGF-1#4 | |
| IGF-1 -> VM202 | 50 μg pCK-IGF-1#1 + | 100 μg VM202 |
| | 50 μg pCK-IGF-1#4 | |
| VM202 -> VM202 | 100 μg VM202 | 100 μg VM202 |
| VM202 -> IGF-1 | 100 μg VM202 | 50 μg IGF-1#1 + |
| isoforms | | 50 μg IGF-1#4 |

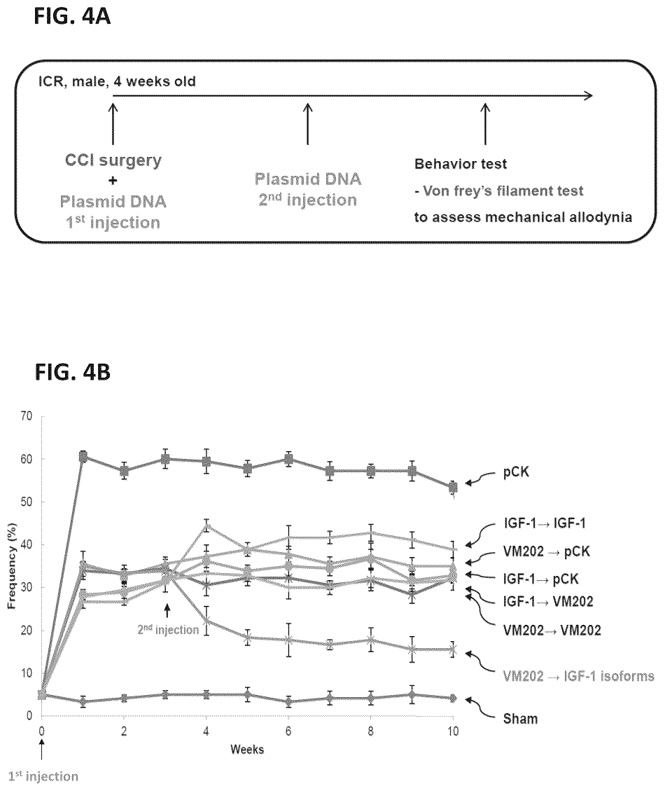

Therapeutic effects of the injections were tested by measuring the mechanical allodynia on a weekly basis by Von Frey's filament test throughout the experiment. The experimental procedure is summarized in FIG. 4A. Briefly, animals were placed individually in a cylinder on top of a metal mesh floor for adaptation. The frequency of mechanical sensitivity of mice was assessed by stimulating the hind paw using constant thickness of the filament (0.16 g). The test was repeated weekly thereafter.

The results are summarized in FIG. 4B, providing the frequency (%) of paw withdrawal measured in each group on a weekly basis. The result confirms that injection of IGF-1-encoding DNA construct (i.e., IGF-1 #1 and IGF-1 #4) or injection of HGF-encoding DNA construct (i.e., VM202) provides significant reduction in paw withdrawal frequency as compared to vector alone (pCK). Furthermore, it was demonstrated that injection of IGF-1-encoding DNA constructs (i.e., IGF-1 #1 and IGF-1 #4) after injection of an HGF-encoding DNA construct (i.e., VM202) further reduces paw withdrawal frequency (VM202->IGF-isoforms). This additional reduction by 2^{nd} injection was not observed in the group where IGF-1 #1 and IGF-1 #4 was injected before the 2^{nd} injection of VM202 (IGF-1 isoforms->VM202) or VM202 was injected before 2^{nd} injection of VM202 (VM202->VM202). Therefore, injection of VM202 followed by injection of IGF-1 #1 and IGF-1 #4 (VM202->IGF-1 isoforms) provided the most significant reduction in paw withdrawal frequency.

### 6.6.3. Example 3: DNA Constructs Capable of Expressing Both IGF-1 Isoform #1 (Class I Ec) and Isoform #4 (Class I Ea)

Since a first IGF-1-encoding DNA construct expressing IGF-1 Class I Ec (IGF-1 #1) and a second IGF-1-encoding DNA construct expressing Class I Ea (IGF-1 #4) together had statistically significantly greater ability to reduce mechanical allodynia in the behavioral experiments described above, we constructed several plasmids designed to simultaneously express both the IGF-1 Class I Ec (Isoform #1) and Class I Ea (Isoform #4) isoforms through alternative splicing of the RNA transcript. In particular, DNA constructs were generated to comprise sequences for exons 1, 3/4, 5 and 6 and introns of IGF-1 gene or their fragments. Several DNA constructs including different variations were generated and tested for their capability to express both IGF-1 Class I Ec isoform (Isoform #1) and Class I Ea isoform (Isoform #4).

Each plasmid was constructed using pCK as the plasmid backbone to contain an insert operably linked to the pCK expression control sequences. The insert was created by concatenating (1) a first polynucleotide encoding human IGF-1 exons 1, 3, and 4 (SEQ ID NO:1); (2) a second polynucleotide, either the IGF-1 intron 4 (SEQ ID NO: 2) or a fragment thereof, (3) a third polynucleotide encoding exons 5 and 6-1 (SEQ ID NO: 3); (4) a fourth polynucleotide, either intron 5 (SEQ ID NO: 4) or a fragment thereof; and (5) a fifth polynucleotide encoding exon 6-2 (of SEQ ID NO: 5), in which the first polynucleotide, the second polynucleotide, the third polynucleotide, the fourth polynucleotide and the fifth polynucleotide were linked in sequential 5′ to 3′ order. The plasmids differed in the size of the fragment of intron 4 and/or intron 5. Specifically, SEQ ID NO: 6 provides the nucleotide sequence of the intron 4 fragment used in vector pCK-IGF-1X6, and SEQ ID NO: 7 provides the nucleotide sequence of the intron 4 fragment used in vector pCK-IGF-1-X10. SEQ ID NO: 8 provides the nucleotide sequence of the intron 5 fragment used in vector pCK-IGF-1X6 and pCK-IGF1X10.

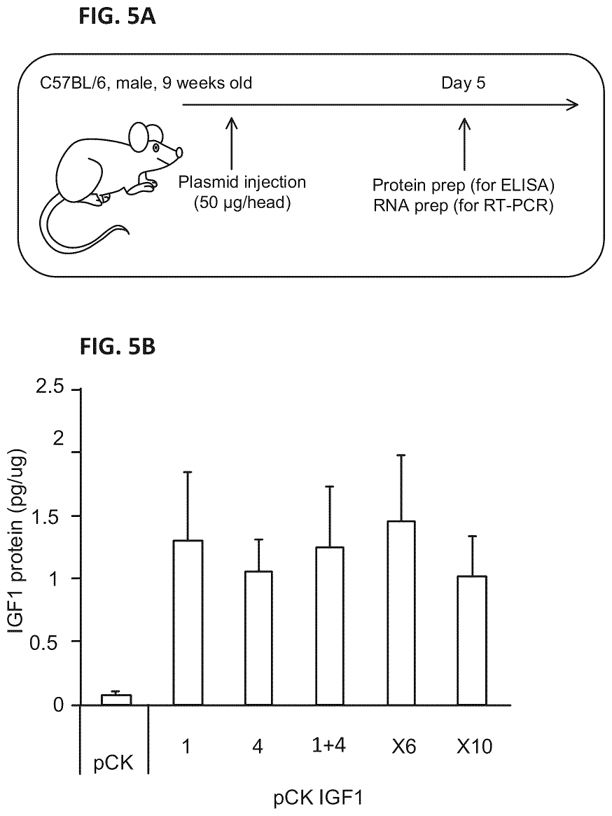

To test expression of Isoform #1 (Class I, Ec) and Isoform #4 (Class I, Ea) from the various constructs in vivo, 9 week old male C57BL/6 male mice were injected with 50 μg plasmid in their T.A. (tibialis anterior) muscle. Their T.A. skeletal muscles were obtained 5 days after the injection. The skeletal muscles were then homogenized in a lysis buffer containing protease inhibitor, phosphatase inhibitor cocktail (Roche Diagnostic Ltd.), and PMSF (Sigma) using polypropylene pestles (Bel-Art Scienceware). The samples were centrifuged at 12,000 rpm for 15 minutes at 4° C., and the supernatants containing total protein were subjected to human IGF-1 ELISA (R&D Systems) as set forth in the manufacturer's protocol. The level of IGF-1 detected was normalized to the total amount of protein extracts from the tissue, as measured by BCA protein assay kit (Thermo, IL, USA). The experimental procedure is summarized in FIG. 5A.

As shown in FIG. 5B, the total expression level of human IGF-1 proteins in mouse T.A. muscle was determined by ELISA. Regardless of whether the mouse received 50 μg of construct expressing a single isoform (“1” (Class I, Ec) or “4” (Class I, Ea)), 25 μg of a first construct expressing isoform #1 (Class I, Ec) plus 25 μg of a second construct expressing isoform #4 (Class I, Ea) (“1+4”), or 50 μg of either construct expressing both isoforms, pCK-IGF-1X6 (“X6”) or pCK-IGF-1X10 (“X10”), the total expression levels of human IGF-1 protein were similar.

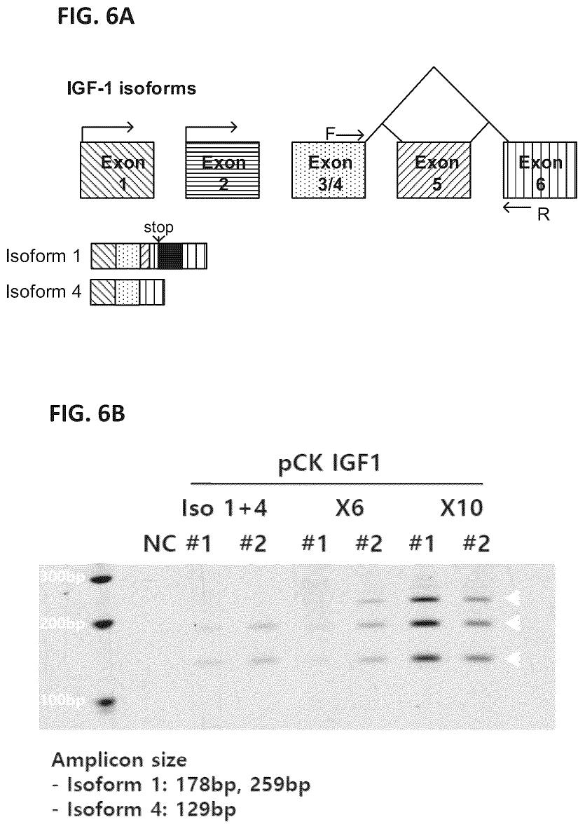

We used RT-PCR to determine whether the constructs expressed mature transcripts for both isoform #1 and isoform #4 simultaneously. RT-PCR reactions were performed with a forward primer (F) that binds to exon 3/4 and a reverse primer (R) that binds to exon 6. As further explained in FIG. 6A, the RT-PCR of a transcript for Isoform #1 (Class I, Ec) would generate two amplicons—178 bp amplicon and 259 bp amplicon, whereas the RT-PCR of a transcript for Isoform #4 (Class I, Ea) would generate a single amplicon of 129 bp.

For RT-PCR, skeletal muscles were collected, mechanically homogenized using polypropylene pestles (Bel-Art Scienceware), and extracted in RNAiso plus (Takara). Quantification of RNA was done by using a nanodrop instrument. Equal amounts of RNA were used to synthesize cDNA using Reverse Transcriptase XL (AMV) (Takara), and PCR was performed using the forward (TGA TCT AAG GAG GCT GGA) (SEQ ID NO: 40) and reverse (CTA CTT GCG TTC TTC AAA TG) (SEQ ID. NO: 41) primers indicated in FIG. 6A.

As illustrated in FIG. 6B, pCK-IGF-1X6 and pCK-IGF-1X10 expressed mature transcripts for both isoform #1 (178 bp and 259 bp bands) and isoform #4 (129 bp band). Expression of mature transcripts for both isoform #1 and isoform #4 were not detected from constructs other than the pCK-IGF-1X6 and pCK-IGF-1X10, which data are not provided herein.

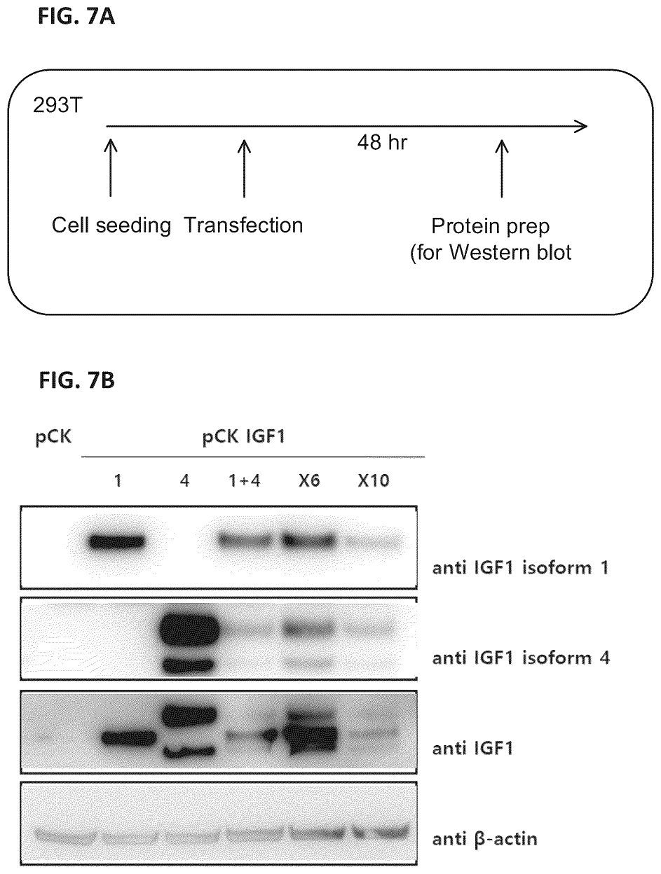

In order to confirm that the two isoform transcripts were both effectively translated into protein, we transfected 293T cells with pCK-IGF-1X6 or pCK-IGF-1X10, as illustrated in FIG. 7A. For immunoblotting, cells were prepared 2 days (48 hours) after the transfection of plasmid DNA, followed by lysis using RIPA buffer with protease and phosphatase inhibitor cocktail (Roche Diagnostic Ltd.). Equal amounts of protein were separated on 10% SDS-polyacrylamide gel, and transferred to a western membrane (PVDF). The membrane was blocked with 1% BSA (Invitrogen-Gibco) in TBST (20 mM Tris-HCl, pH 7.4, 0.9% NaCl, and 0.1% Tween20) for 1 hour and probed with primary antibodies diluted in blocking solution at 4° C. overnight. Primary antibodies used to examine the level of IGF-1 isoform 1 and isoform 4 were provided by Abclon (Korea), and those for IGF-1 and 3-actin were purchased from Abcam (UK) and Sigma-Aldrich (US). After washing with TBST, membranes were incubated with HRP-conjugated goat anti-mouse or rabbit IgG secondary antibody (Sigma) at room temperature for 1 hour. The blots were then washed three times with TBST, and the protein bands were visualized with the enhanced chemiluminescence system (Millipore). 3-actin was used as a loading control.

Western blotting data shown in FIG. 7B confirm that pCK-IGF-1X6 and pCK-IGF-1X10 plasmids express both isoforms of IGF-1 at the protein level.

### 6.6.4. Example 4: Therapeutic Efficacy of Co-Administration of HGF-Encoding DNA Constructs (VM202) and IGF-1-Encoding DNA Construct Expressing Both IGF-1 Isoform #1 (Class I Ec) and Isoform #4 (Class I Ea) in a Murine CCI Neuropathy Model

As discussed above in Examples 1 and 2, concurrent or serial administration of VM202 and IGF-1-encoding DNA constructs—pCK-IGF-1 #1 and pCK-IGF-1 #4, (i.e., an IGF-1-encoding DNA construct capable of expressing IGF-1 isoform #1 or IGF-1 isoform #4) —provided significant reduction in mechanical allodynia in a mouse CCI model of neuropathy.

We tested whether the same effects can be provided using a dual expression DNA construct capable of expressing both IGF-1 isoform #1 and IGF-1 isoform #4, generated in Example 3. Specifically, pCK-IGF-1X6 and pCK-IGF-1X10 were tested in combination with VM202 in a mouse CCI model of neuropathy.

TABLE 3

| | IGF-1-encoding DNA | HGF-encoding | Other DNA |
|---|---|---|---|
| Group ID | construct | DNA construct | construct |
| pCK | None | None | 200 μg pCK |
| VM202 | None | 200 μg VM202 | None |
| VM202 + | 50 μg pCK-IGF-1#1 + | 100 μg VM202 | None |
| IGF-1#1 + 4 | 50 μg pCK-IGF-1#4 | | |
| VM202 + | 100 μg pCK-IGF-1X6 | 100 μg VM202 | None |
| IGF-1X6 | | | |
| VM202 + | 100 μg pCK-IGF-1X10 | 100 μg VM202 | None |
| IGF-1X10 | | | |

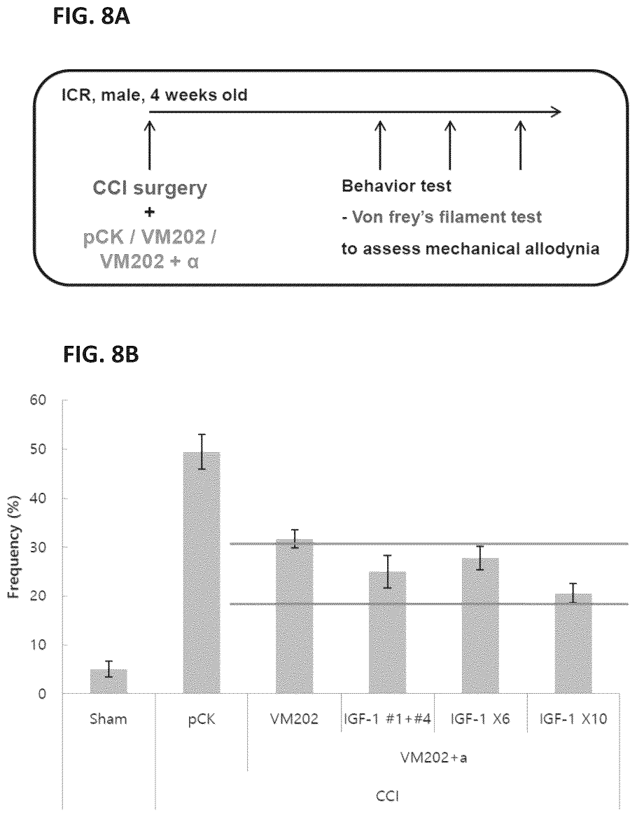

CCI mice were divided into five groups and administered with total 200 μg of DNA construct (as provided in TABLE 3) by intramuscular injections on the day of CCI. The pain sensitivity towards mechanical stimuli was measured at appropriate times by Von Frey's filament. Each group consisted of 6 mice, and more than 2 independent experiments were performed (mean±SEM; *, p<0.05;**, p<0.01; ***, p<0.001). One week after CCI surgery, the development of mechanical allodynia was assessed using Von Frey's filament, and pain symptoms were evaluated weekly. The experimental procedure is summarized in FIG. 8A.

Paw withdrawal frequencies measured one week after CCI surgery are provided in FIG. 8B. The data demonstrate statistically significant reductions in mechanical allodynia after simultaneous intramuscular injection of VM202 and constructs encoding IGF-1 isoform #1 and #4 (i.e., IGF-1 #1 and IGF-1 #4; IGF-1X6 and IGF-1X10). In particular, the effects on mechanical allodynia was better when the mice were administered VM202 simultaneously with two IGF-1-encoding DNA constructs, each encoding IGF-1 isoforms #1 or #4, or with the dual expression construct pCK-IGF-1X10. These effects were consistently observed until the last measurement at four weeks after CCI surgery.

### 6.6.5. Example 5: Therapeutic Efficacy of Co-Administration of HGF-Encoding DNA Constructs (HGF728) and IGF-1-Encoding DNA Construct Expressing Both IGF-1 Isoform #1 (Class I Ec) and Isoform #4 (Class I Ea) in a Murine CCI Neuropathy Model

As discussed above in Examples 1-4, concurrent or serial administration of VM202 and IGF-1-encoding DNA constructs provided significant reduction in mechanical allodynia in a mouse CCI model of neuropathy. Therapeutic effects of a different HGF-encoding DNA construct, HGF₇₂₈, were further tested in combination with IGF-1-encoding DNA constructs.

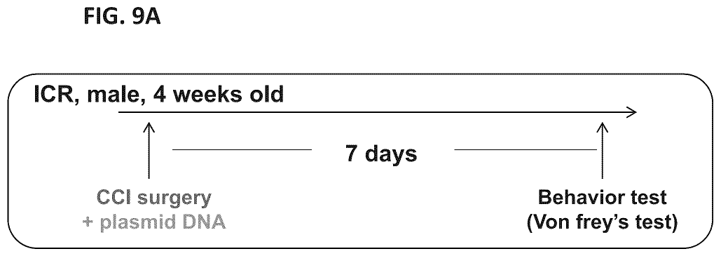

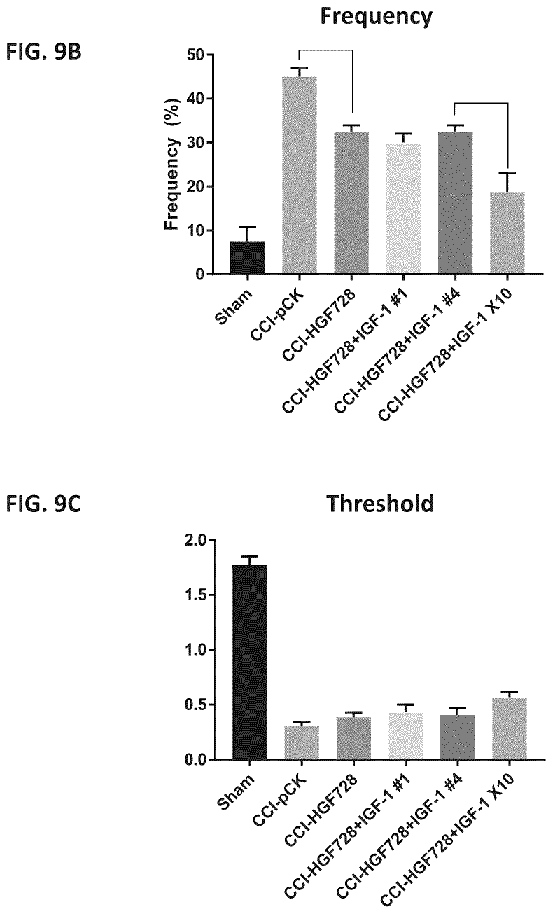

Specifically, CCI neuropathy mice were generated as provided in Example 1 and divided into five groups. As schematized in FIG. 9A, a total of 200 μg of plasmid DNA was intramuscularly injected on the day of CCI surgery. DNA constructs administered in each group are summarized in TABLE 4.

TABLE 4

| | IGF-1-encoding DNA | HGF-encoding | Other DNA |
|---|---|---|---|
| Group ID | construct | DNA construct | construct |
| CCI-pCK | None | None | 200 μg |
| | | | pCK |
| CCI-HGF728 | None | 200 μg pCK-HGF₇₂₈ | None |
| CCI-HGF728 + | 100 μg pCK-IGF-1#1 | 100 μg pCK-HGF₇₂₈ | None |
| IGF-1#1 | | | |
| CCI-HGF728 + | 100 μg pCK-IGF-1#4 | 100 μg pCK-HGF₇₂₈ | None |
| IGF-1#4 | | | |
| CCI-HGF728 + | 100 μg pCK- | 100 μg pCK-HGF₇₂₈ | None |
| IGF-1X10 | IGF-1X10 | | |

One week after CCI surgery, the development of mechanical allodynia was assessed using a Von Frey's filament test. Briefly, animals were placed individually in a cylinder on top of a metal mesh floor for adaptation. To examine the frequency of mechanical sensitivity, mice were assessed by stimulating the hind paw using constant thickness of the filament (0.16 g). For mechanical threshold, mice were stimulated with various thickness of the filament (0.04˜ 2.0 g). The test was repeated weekly thereafter.

Paw withdrawal frequencies and mechanical threshold measured one week after CCI surgery are provided in FIGS. 9B-9C, with FIG. 9B providing the frequency (%) and FIG. 9C providing the threshold of paw withdrawal. All values are presented as mean±standard error mean (SEM) from three independent experiments. Differences between values were determined by one-way ANOVA followed by Tukey's post-hoc test or Bonferroni's multiple comparison test.

The data show that injection of pCK-HGF₇₂₈ significantly reduces paw withdrawal frequency and increases paw withdrawal threshold. When the animals were treated with pCK-HGF₇₂₈ and pCK-IGF-1X10, greater reduction was observed in paw withdrawal frequency and greater increase was observed in threshold of mechanical sensitivity, which were statistically significant. These results demonstrate that therapeutic effects of pCK-HGF₇₂₈ can be also enhanced by co-administration of pCK-IGF-1X10. These effects were consistently observed until the last measurement at two weeks after CCI surgery.

### 7. Incorporation by Reference

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

Sequence Listing

| SEQ ID | | |
|---|---|---|
| NO. | Description | Sequence |
| SEQ ID | IGF-1 (exon | ATGGGAAAAATCAGCAGTCTTCCAACCCAATTATTTAAGTGCTGC |
| NO. 1 | 1, 3, and 4) | TTTTGTGATTTCTTGAAGGTGAAGATGCACACCATGTCCTCCTCGC |
| | | ATCTCTTCTACCTGGCGCTGTGCCTGCTCACCTTCACCAGCTCTGC |
| | | CACGGCTGGACCGGAGACGCTCTGCGGGGCTGAGCTGGTGGATG |
| | | CTCTTCAGTTCGTGTGTGGAGACAGGGGCTTTTATTTCAACAAGC |
| | | CCACAGGGTATGGCTCCAGCAGTCGGAGGGCGCCTCAGACAGGC |
| | | ATCGTGGATGAGTGCTGCTTCCGGAGCTGTGATCTAAGGAGGCTG |
| | | GAGATGTATTGCGCACCCCTCAAGCCTGCCAAGTCAGCTCGCTCT |
| | | GTCCGTGCCCAGCGCCACACCGACATGCCCAAGACCCAGAAG |
| SEQ ID | IGF-1 | GTAAGCCCACCTGGGTGGGATCCAGCCATCCTCAAGTGGTCTCTC |
| NO. 2 | (intron 4) | TCTTGTGCATGTGGGTGGGCCAAGCAGAAATCCTGCCCCATAGTC |
| | | TCCTGGCTTACAAGTCAGAAAAGCTCCTTTGCACCAAAGGGATGG |
| | | ATTACATCCCCATCTCTTTGGTCACTCTGCATTGCAAATTTCCCCT |
| | | CCCACCGCTATGGACGATGTGATGATTGGAAGATGTTACAAAACA |
| | | GTGGCTAAACAAACATGGGCTTTGGTGTCAGACAAAAGTGAAGT |
| | | CCTGGCTTTCTCACACACCAGCTTAGAGCCCTTGGCAAATAATGT |
| | | GATGTACCCAAGCCTCAGTTTCATCAGTAACATTGGGATAATAAT |
| | | AATATCTACCACATCAGTTTGTTGTCAAAATTAAGTAGCTCATGC |
| | | ATATACTTTGAGATGCTTTTCACATGCCTGCATAAAGTAATTGTTG |
| | | GACCATCGTTAATGTCTGCCATAATTGCACTTAATAACAAAGCTT |
| | | GTAACCTTTCAAGTTCTGAGATTCTACAATCTTCCAAAGAAAATA |
| | | AAAGGCTAATGGGAACTATTCAAAATTCATATTCAGTAGCAAGCA |
| | | TAATTAAACATGAAACATTAAAAATAGAAATTTCTGTTTGGCTAT |
| | | AAGAATGCCTAGACATTTGTAATGATCAAAATCTGCAGGCATCAT |
| | | TTTCTAAGAGCTAGACTGTAAACAAACCTCAGAGGTACCAACTAT |
| | | GCCATCAGTAGTACATAAAACATCTGATGCACATTTAGTCACTTG |
| | | ATCGATTTCTCTTGAATGAGTGAACGAATGAACAAATGAATATAA |
| | | GAGATTAAAATTTTAGCCATTAAGTAGAAAGAATAAGAACTAAA |
| | | GAGAAGGTAAAGGAGGAAAAAGAGAAGGCAAGGAAGTTGAGTA |
| | | AGGGAAGAAATAGCTCTCGTTTAAGTATTTTGGGGACTCTGTTGA |
| | | AAAAAGAAATGCCAACATGTGGTTTTAATCTTTGGAGCTAGAACT |
| | | AATAATATTGTGCAAAAGCACAAGATGAGAGATCAAGAAGTTCA |
| | | CCATGACACCTTCGCTGCTTCCTGGTCTTAAACCTCAGCTGAGGCT |
| | | GGAAGAGGACCATGGTGGCTTATTGGAGATGTGACCCCAGGGAG |
| | | CCCCTCTGAAGGATGGAAGGGGACTGGGCAAGACCCAACACACA |
| | | CAGAACACAGTAGCCACTGGCCAGGCAGGAAGCAAGGATCTCAG |
| | | AAAAGACTTTTAGGTGAATGTGGCAGGAAAGCGTGCTTGCTGGG |
| | | GCAAAGGCAGATTCATTCTTTCTCTTCCCAGGTGACCCAGCGCCT |
| | | CTTGGTTTCTAACTGGGGAGGGGGTAGGTGTCAAGAGATGAGTCC |
| | | CAAAGTTCTGGAATGGTGGGTCTTGTGACTGAGGTCTAGACCCCT |
| | | CTCCAGCATGAGTGCTGTCTCCTGCATCATATGGAGCCTGGGCAT |
| | | TCTGAGCTCATTCAAAGGGACACCATGGGAACCACTTGTTCTCAA |
| | | TGCAATTATTTTTGTGATGTTTACAG |
| SEQ ID | IGF-1 (exon | TATCAGCCCCCATCTACCAACAAGAACACGAAGTCTCAGAGAAG |
| NO.3 | 5 and 6-1) | GAAAGGAAGTACATTTGAAGAACGCAAGTAG |
| SEQ ID | IGF-1 | AGGACAGGAGGATTAAACAGACAGAGGCAAGGATGATGAGAGA |
| NO. 4 | (intron 5) | GGAGCAGACAGCAAGAATGAAAAGCAGAAAATACAATAGAGGA |
| | | AATGAAGAAAAGTAGGCCTGCTGGAGCTAGATGATGATGTGATG |
| | | GAAATAGAAGTAACCTTTTAGAGAATCTCGCTAAGAAACATGGA |
| | | GAAAACGGAAAAGAAAAATGTAATGCCCTAGAAAGCGCAAAGA |
| | | AAGACAGTGGCAAAAATGAAAAAAAAAAATAAAAATTATAAAA |
| | | GAGGCAAAAAAAGACACACTATTCTCTGCCTCTAAAACACAATTA |
| | | AATAAAAGAATTTAAATAAAAATTAAGGCTTCTATATGCATTTTT |
| | | AAATTTTGTATGAATCTGTTATGGAAGAATTGCCTATGTCAATAT |
| | | ATGTTCAGAGTTAAATATTAGCCCCAAATGCTCAGCAAGACTGAA |
| | | TTGTGTCATAGAAGTTCCCAGATTCCCTTTTCCCGCAATGTCATTG |
| | | GAGGCTGCATTTCTTAGTCAAGTCCAGGGTTTAGGCCAAAGGGCA |
| | | TCCGGTATTGCCTAAAACCCTGTGAGGTCTGTGAGGTAACTTTTG |
| | | AGAAGAGGTCACTGCACTCTTCATCTTTTTTGCACTTTGGAATCAG |
| | | ATATAAAAGATGTATAAGTTTGCTAGGGCTGCCATAACAAAGTAT |
| | | CATAGGCTAGGTAGTTTAAACCACAGAAATTGATTTTTTCATAGT |
| | | TCTGGGAGTTGAAAGTCCAAAATCAAAGTATCAGCCCTTGCAAGG |
| | | GCCTTAGAGAAGGCTCTGTCATGGGCTCCTCCCCTCGGCTTGTAG |
| | | GTGGCCTCCTTCTTCTCCCCCTGTGTCTTCACTTCATCTTCCCTCCA |
| | | TACATATCTCTGTGTCTAAACATCCTCTGTGTGAAACAACACCAG |
| | | CCAGGTTGGATTTGGGCCCACCCCACTGACCTCATTTTAACTTAAT |
| | | TATCTCTGTAAAGACTCTGTCTCCAAATACAGTCATATTTTGACGT |
| | | ACTGGGAGTTAGGGCTTCAACACATGAATTTGGACACAATTCAGC |
| | | CAGTGACAGAAGACTTCTGATCTCTGATGATAACCACTGCATTTT |
| | | GATTACAGCTCCTAGAAAACACTCCCCTCCACCACCCCACCACAG |
| | | ATCTATTTTTATATCTGAAACCCTGAGTTTCTGCTCCATGAGAACC |
| | | CCAGGAACATACTATGTTAGATCTGGAAGAAGCCTCAGAAATCCC |
| | | CTTATTTTGAAGACTAGGACACTGAGATCCAGAAGTGGGTAAAG |
| | | ATGTGCTTGGGTTCTAAGCTGCTCTTCTTTTGGCCAGGAGACAAC |
| | | AGCACATAATCAAAGTGGGTCAACTAAGAAAGAATTCCAGAAGG |
| | | AAAAGAGAGGGCAGAAATGAAGGGAGAGAATGAGAGCAAAAGT |
| | | GCTGGATTTCCCTGAGGGTGAAGAAAAGTTAAATAGAATCACAG |
| | | AATTCAGATTTTAGAGATCTTCTCCTTCAGATCCCTTGGTTTAATC |
| | | AGTAGGATTGGGGTCTTCATAGATAATAAAGCAAAAACTCTCGCC |
| | | ATCCTCCAAGTTGTGAATTAGAAGAGCTGAGAAAGGGTACAAGA |
| | | CGGAAGTTCTCTACCAAACAAATGGTGACATTTTGGGGTAAGAAT |
| | | ATGACTAACCCAGAAGTGAAGCATTTCATCCAAGTAGTCTATTTT |
| | | GAAGATGTCATGGTATAAAGGAACCTCCTTTCTGCCTGGTCCTCC |
| | | ATGCCTCTGCCATGCTTTTTACTCCAGGATCACCCTTTCTAGTGGT |
| | | TCACTGAAAACCCAGGATTACTTAAATATGATGGACATGTTCACG |
| | | GCTCAATCCAGGAGGAAAAGGTCGAACTGAAAGCATGCCAAAGC |
| | | CCCACATGGGAGCCAAGCCACTGCTGCTGTGGTTGCAAAGTGGAT |
| | | CCTGGCTTATCAGAGCAGAGAGAAGCCAGGCTCGTGCCTTAGCCC |
| | | AAGTGGCCAGTCACCTTATTCAGGAGATACTAAGTTCTCCAGCTA |
| | | AGACATCCATGCTTTGGGACCAGCTGCAGACAGAAGCCAATTCCT |
| | | ACTACAACCATCACCTTAGAGTAGCATATAGACACAGATGGCTCT |
| | | TCAAAGGACCACAGTTCCATGGAATAACTAAGAATTCATGTCCTG |
| | | TGGAAAGGTTTGAATAAACTATAATTATACCCAATCATAAATTTC |
| | | ATTCAAGAAGAACTAAAGCAAAGGCAAAGACAGAGAGAAGAAG |
| | | GAAGGAAGGAGGGAGGGAGGGAGGGAAGGAAGGAAGGAAGGA |
| | | AGGAAGGAAGGAAGGAAGGAAGGAAGGAAGGAAGGAAAGGGA |
| | | AGGAAGAACAAAAAGACTTTCTAGTTAAAGAATGCTTAACTAGC |
| | | AAACTATGTACTATAAGACAGTTCTTTTCGGAATGAGTTTTATCA |
| | | ACTCTAAAGCAATTATCTTGAATGCCTACATGTGATTACTGAATA |
| | | ATATGAACCAAGAAAACAGAAAGAATCTATATTATCTTTCCATTT |
| | | CCTTCTTTCCAGTATCAATACCCAAGCCTCTAGTGATACATGGCAT |
| | | ATAATGTTGGATGGATGGATGGATGGATGGATGGATGGATGGAT |
| | | GGATGGATGGATGAATGGATGGTTGGATGGACAAATGAGTAACA |
| | | TAGGCTGATGAATAGTGGTAGAAAGACACACCATAAAAACAAGT |
| | | GGCACTTCTGAGATGAAATGATTCCTATTCTCCTACACAAGACAG |
| | | TGAGGCAAGTACAGAGTAAAAAAGGAAAGGCATAGGAGCTATGC |
| | | TTATACAAGTATTGTATGTTTGGAATTTCCTTCGCTGGCCAAATTG |
| | | AAATTGTTCAAGGACCTATTGCTACAGGTGGCAACTGGCTAAGAA |
| | | TTTCATAGTGAATATTATACACCTATTACTCCCCTTAATGTTTCTT |
| | | TGAAGTAAGCAGAATATTAATAATCATTTAAAATTCCAGTGTTTC |
| | | AACTTCAATTGTTTCCTAGGGCAAATTGATAATTGTGTGTAAAAC |
| | | TAATTGGAATATGTATGGAATAATCATCCTGAAATAAAATTGGTG |
| | | AAAAGTATTTGTTATTGGGCATCTACAATGTGCAAACCTCTGTAC |
| | | TAGGCATGAACAAGAGTTATAAGCATTGGAGAGGCTAAAATATA |
| | | GTCCTTAAGGCTGGGCACAGTGGCTCATGCCTGTAATCCTAGCAC |
| | | TTTGGGAGGCCAAGGCGGGCAGATTGCCTGAGCTCAGGAGTTCA |
| | | AGACCAGCCTGGGCAACATAGCGAAACCCCATCTCTACTAAAAA |
| | | TACAAAAAAATTACCTGGGCATGGTGGCACGCACCTGTAATCCCA |
| | | GCTACTCAGGAGGCTGAGGCATGAGAATTCCTTGAACCTGGGAG |
| | | GCAGAGGTTGCAGCGAGCCGAGATCCTGCCGCTGCATCCCAGCTT |
| | | GGGTGACAGAGTGAGACTCTGTCTCAAAAAAAAATTAAATAATA |
| | | AATAAATAGTAAAATACAGTCATTAAGAGTACAAAATGTAGATT |
| | | CAGACTACCTGGGTTCAAATCTTGGCTCTTACTTGCATTGTGGCTT |
| | | TGGGCAGATCATGTAACTTATGTGTGCCTCAGTTTCCTCATCTGTT |
| | | AAATAGGGGCAACAACTGAATCTACCTTATTCAGTTGTTGTGAGG |
| | | GTTTATTGAGATTGTGTGTGTGTATGTGTGTGAGTGTAGTGTGTGC |
| | | ATGTGTGTGTCTGTGCAAGGAGTGGGAGGTGTATATTCAGAGACA |
| | | CATATTACAGCACTTAAAATGGTATCTAGCACTTAGTAAGCATTA |
| | | TTCAAGTTTTAGTTAACATTATTTTACTTACCTCTGAAAATTGGAG |
| | | CTATGTGAAAAAGAAGTTGGTCTCCTGAAGTAGAAGCCAGTCTTG |
| | | TGTCACCAAAAACTTCAAGCCCAAGCTTGCCAACGCTTTTCCATG |
| | | ATGTGGTAGTAGAGTTTCAAGCATGTGGTAGGATAAGAGAACTC |
| | | AATGACCTAAGAACCATTCCAACCCAGAGAACCCCTGGTTCTATG |
| | | AATAATTCCAACTTAAATAGGTAGCTTGGCTCTCCCAAGTGAGAG |
| | | CCATTGCTTCTGTTTCCGGGTCATATAATGAACTTTCAGAAAACC |
| | | ACCATTTTTCTCAACCAGTTAAAATTAAGTGTAATACGTGCTTTCA |
| | | TTTCATGGTGCCTGGGGAAAATTTAATTGTAGTATGAACTCCAGT |
| | | TATTGGTAGTCTTAAGTAAAATTGCCAAAATAAATAGAAATGCAG |
| | | GATATTTCTGGGCTCACACAGCTTCCGGGACACTTTAGTTTCTTGG |
| | | GCTGCCAATCCAGTGCCTTTCACAAGCATTTGATCTTTTTTCAAAC |
| | | ATCTCTTGAAAACAAACAAAACCTCACACAGCTTCTAATGTGTGC |
| | | ACTGTTCGAATGTAAGGGTGGAAAAGGAGGCAAAGAAATGAGCT |
| | | CCCAAAGAGCAATTCCCCTTCTCTCGCCTCCATCCCTTGACGACCT |
| | | CCCTCCCACTAAAGGGAAACATTGTTTTCTTAGGTAATAAATTCT |
| | | GCAATTTCTCAAGTCCATTAACATCCACTGGGCAAGATGAGATCT |
| | | ATTCTTTTTATTTGCCCATAGGAAAAGAATAGTGCTTTTTTGCAAT |
| | | ATTCACTAGATAACACAGAGTTGACTTTTAATCCAAGGGCAACAT |
| | | TGATAGTCTCTAGTTAAAGGGGAAGCCTTCAGGAGCAATGAAAA |
| | | GATTAATAGTTTTAGATGAAGCAGAATCCAAATCCCTTTTTATGA |
| | | GTTTTGAAATATCCAGTTTGTATGCTCACCTCAATACTTAAAGCCC |
| | | AGTTACTGATTCCTTTGGCCTAAGCAAGACAGGTCAATTTTTAAA |
| | | GAGGGAGTAGCTGAGGTTAGCAAAAATTCTCCAGGTCCACAAAA |
| | | CTTCCAGACCTGCAAGGTGAAAATCAGCTTTTCTGTCATCCCTAA |
| | | AGGCCTAACTGGAATCAGAACTTTTCCCTGATGCCCACATATTTG |
| | | GAGGTCCTTTTTTAATGGGACTCCTTAATGCCTTTAGTGCCATCCC |
| | | ATTTTCATCCAGTGTCCAAAAGAAATGATTTAAAAATATAAACGT |
| | | ATGTTTAAATTCCAGAAGAGAGAAATGGAGATTGAGAACAATAG |
| | | GGAAATGATGAGAGCTATGGGAAAAGAGGTTTATGAGTCCATGT |
| | | CTGATTCTTCCAGAGAGCCCCTAAGAAAGTTCTTATCATACCAGG |
| | | AACTCAATTATAACTTTCATTGCCTATTGTTAGATGAGTAACAGG |
| | | AGCTAGAAAACATTTTGGAAATTCCCATCTTTATTTTTTTAACTAA |
| | | TATGATTATAGTTTTAAGAACCATTGGTCAAGAAGCTAACTTTTT |
| | | AAAAAGTGGAAGTATGATGGTTAGAAATAAGAATGCTAAAGGTG |
| | | CATCAAGCTGATTTTAATTCTAAATGTCCTTGGCAGCAATTTAGA |
| | | ATCTGTAATAAACTACACCAAACAGTTTTGAGGGGAAGGGGATT |
| | | AGTTTCTCCCCTTCCTTCGTGTGTGTGTGTGCGCGTGTGTGTGTGT |
| | | GCACCTTTGTGTTCTAGCATTGTTGCACCCATTACAGAGCTGGGG |
| | | GGAACTATTTTCCAAAATTATAGGTGAGAACAGTTTCTTGGATTG |
| | | TCTTTCAGTGAAGGTAAATTCCTCTGTAAAAACTAACCATCATTC |
| | | AGTAAAAACTGCAGGATTCCTTTGTCTTCTCAAAAGCCTGTTTCTC |
| | | ATCCTAAATTAAAAATTATTCAGGAAATAGAGAGGACATTATTGG |
| | | AGGGGTGGAAATAAGTTGGTTTTCTTTTTATTGTATCTTTTGAGGA |
| | | TCCAGGGACTTCTACCATTTCCCATCTAACATACAGAGAAGGATT |
| | | CTCTAGGTCCCTGTCTATAGACTGCAGTAACTTTCCTATAGAACC |
| | | AATTTGCAATTTTAGAAATTTCTAGGTCTAATTATTGACCCATTAC |
| | | AACCAAAGGTCAATGCATCCAGCCAATCTTCCTTCTATCATCCCC |
| | | TGCCCTTACTTCTATTAGGGACTGGGATTACAGGCAAAACCCATC |
| | | AAATGCCTCTTCTACCACTTTCCCATTTCTTAACCATTAGCCTCTA |
| | | ACTTCCTCTATTCAGTTTCTCATATGCTTTCATGCCCATTGGGTCA |
| | | GATAAAGGAACATTCATTTATTTGAGTAGGCATCTGTTATGATCA |
| | | CTCCGGAAAAAAGATGACAATGGGTTACCTTGTCCTCCTGGGCTT |
| | | CTCTAACTGACATGGTCAAAATGCCCATATGAAGATAAGATGTTA |
| | | AGAGCAAGATTTATGAAAAGCTGAGTATGATGGCAGCTCTTGTCT |
| | | CATAAAATAACTCGAAAGTTCCCAGTGAAAGACCAAGAAATTTT |
| | | ACATCAAACCCAAACCGGCCAAATGGTCCAAGCTTCCAAGCTGG |
| | | GATCCATGGCTAAAGTTTCTACAAAATTCTGGGTACAATGTATAA |
| | | ACATTCACTTGGGGCTTTCTGTCTAGCCAGCACCAAGAGGTCAAG |
| | | TAATCAAGGACCAACTAGCCCTGCCATCTGTGAAAATATGTGCTA |
| | | TTTTCACGGCTTTAGTTCACAATTATGGCAAGACAAAAGTTCCAA |
| | | ATAATTAGGAGCAAGACCATGGCAGGTTGACGGTTGAGTAAGGT |
| | | TCTCAATCAGCCGACAATTGTAGAGTTGGGGATGTGCAATGTTTA |
| | | TGTCATGGTGTAAGTATGTGGCATGCTTGACTAGCTTGTGAGGCA |
| | | CTGGAAGACTAGAAGGAATGAAAAATATGAATGAATCAATAAAT |
| | | GCATAGTATAATTACTGTTATTTTGTCAGTATTGTTTTACCTAGGT |
| | | CACTATTGAATGCTCTGATTTGTCTCTTTATAAATAATAATATGTT |
| | | TTCTTCTTCAAAAGAACACTAGGATGAAGGTAGAGGTGCTTTTGG |
| | | CACAATGCCACAATTCTGATTTTTTTAAAACTGTATGCATGCATA |
| | | AAATGTTCTTGAGCCATTCTCTGCCTTGGAATAGCACTGGCTGGC |
| | | ATTCTGCATGTTTACTTTTATATGCTGAAGGCCCCCATCAACCTCA |
| | | AACAGAGGCAAATCAATTTAACTTCTCATAGTGTTATTTTGTTCAT |
| | | CCTAAAAGTTCAAGAGAGCCTTCCAAACTTCCAAAATTTCTCTCA |
| | | ATTCAGTGAGGAGGAAAATTCAGAACACAGCATTTGAATGTTCTG |
| | | CCCAGATTTGTCACACACACAAGGAATGAGTGAAAGAGGGCAAC |
| | | ACCCTTTCCTCCTAACCCTGTGAACTCATCACTATTGCATTGAAAT |
| | | GACACCAAAAGGTAAAAACCCTAGGCCTCACATCTCCCAAGAAC |
| | | ACTGCAATAGGAGTTACTGCATACACCAGTTTAAGTAACTCTAGC |
| | | ATAAATTGTATGTCAGATGAAACAATGGCATTTTGGAGGCTTAAG |
| | | AGAAAAAGAATAATCAAATCCAGTTTTTAGGTACTAATGTGCTGA |
| | | ATCTTTAGCACATAGCAGCAAAATTGCTAGAATCTGGTGTTTCAC |
| | | TTTTTAAAATACCACATTTGAACCTTTCAGCAATTCCAAAATCAA |
| | | CTCCCTCTGCGAAAGATAATAAGCTTAAACATTTTTTAAATTTAA |
| | | AAATGTAACACAAACAAACAGCTAAGCAAACAAGCTGCCCATAA |
| | | AATCAACAGTCTGGGGAGCCCTGATCCTGAAGTATTTTACAACAT |
| | | CCTTCATGACTATTAAAGGCAACATAAACACCTCTTGTCAGCAAG |
| | | GGAAACTACCCTTGGCATTTTTTTTTCTTTGTTCCCCAGGCTTTTA |
| | | AACCATTTTGATAGAGATTTTTTACATCACAGGCAGAAATATTTG |
| | | AAATAGAGTCAGGTGGTAGTCTTTAAAAGAGTAAGAAAGTTGCT |
| | | AAGTCAAGATAATCTTGGAATAAAGTCCTCTGATTCCTGGGGATT |
| | | CCTAGGGATGCCCCAGTCACTAGAAAACAGAGCTGTAAGTCCACT |
| | | CTCCCAGCACTCAACGGAGCTCCGGAAACCAAGGAGCTAGCTAC |
| | | TGTTTCCCCACATTCAGCCAGAGAAAGGGCAGCACTCTAGCATGC |
| | | AAACTGCTTTGACAATAGTAACAATTAAAAAGTAAATTAAAAAG |
| | | AATCATAATAGCTGATATTGATTAGGTACTTGCCCTGTGGCAAGA |
| | | GCTATAGGGAATCACCTCATTTAATCTTCACATGAAGCTTGCAGA |
| | | GTGAGTACCACAATTATCACTATTGTATAGACAGGAAAACTCAGG |
| | | CTGAGTATGGCTAAGTGTCTTGCCAACGTCTTGGGCTAACAAGCG |
| | | GTCAAGCAGAATCCAAACCCGAGATAGATAGACCACAGTGTGCT |
| | | AATCAAGCACTGCACTCTCTCCTGCATTTCTTAGTTGATATTTACC |
| | | ATATACAATCTGTCACTTGTATGAGATGGCAGGGGGTTCTGTGCT |
| | | ATTTGTCCTTGTAGAGAATACCACAGGAAGAAAGTAAGCAGCCA |
| | | TGCAATATTTGCTGTTGACCTGAACTCCATTCCATCATTCCTGCAG |
| | | GAAATTCGCATCCATTAAATGAGCATTTCCTGGTTTGCCACTTTGC |
| | | TCAAACACTTTGCTTGGATCTGGAGAGGATATAGAAGTGAAGGA |
| | | AATATGCTACCTGCTCTCAAGGAACTTATGTTTTAGTGGAGAGAC |
| | | AAACATGCAGAATTTACTCTACAGAACATCAATGCTTGAGCAAAT |
| | | GTAGACCCAGAGAGGGCTCTTACAGCACACAAGCCAGAACAGAC |
| | | TGATGGTGCTAACAATTAGGTTCAAGGTTTTTCTAAACAGTAGAC |
| | | TCTCCTGCATACAACTATACCGCATGCCAGGTAAATGACTGAGGG |
| | | TTATTACATCCAATTATAACACCACTGTGATGTAGGTGCTCTTACC |
| | | CCACACTTTCATTTTACAGAAGAGGAAATTGAGGACAGCACAATG |
| | | TAGTGATTATCAAAGGTCACACGACTACTGTGTGGGAGAGCTAGG |
| | | ATTTAAACCAGATGCATAAGATGAGGTCCTCCAAGAAACAGAAG |
| | | ATGAGAAGGTGTTAAATGAGCAGGGGTTTTATTAGGGGGAATTA |
| | | ATGTGTGAACAGAAATAGGGGAGGATAGGCAAAGCCATCAGATT |
| | | GCAAGGCAAGCCTAACCCCAAGGGAAGGAGAGAGAGAGAGTAG |
| | | ATTGGTTGGAAACATTTTTGGTGGGTCTATGGTCTAAGGAAAGTT |
| | | CAGCAAAGTCATCATGGAGTTTTTGAGCCAAAGTTGGGCAATACA |
| | | GTTGCCCAACAAATTTCTGTGTTTCTCAGAAATAGGTCTGCCTCA |
| | | ATGTCCCCACCATACTTGGTCACTGGCTCTTGGGAGGGGCCTGCC |
| | | CTGTTCCAATCCACTAGAGCCAAAGAAGAGCCGTTGTACTGGCAG |
| | | GGGGTGGGGGAATTCCTACAACCACATAAAAAGTGGGGTGAGGT |
| | | TTCCAGAAAAAAACGTGATGCTGGGCTAACCAAAACTGTGTCCA |
| | | GTAAGTACATATCCCTCACTCTGTTAAAGAAGCAGCCACATAAAC |
| | | AAGGAGTACACGTTTCTCAAAATGTGCACCTTGTTCTTTGGTTTTG |
| | | AAGTCACATCCCAAAGTGCTGAGTAGATCGCATGACCCTCGCTTT |
| | | GCCTGGCTGCCAGAGAGGAAAGGCTGATCCAACTCTCCTGGAATT |
| | | TGAACTTGTGATTCCCTGAAGTAAAGAGATATCAAAGTTGATACT |
| | | GAGACATCTAAATCATCCTCCACCATTTCACATGTCCCCAGGCCA |
| | | AGCCAGCAAAATTGCTATAGCACATCCCTTTCAACAGGTAAAGGG |
| | | CTGATATCTGAGCCCTCTTTCCAATCATCCACTGCTCTTTTCTTCTC |
| | | ATTTTGCCCTTTTTGGGAGCAGGTCAATGCTGAGTTAGTACTTTAT |
| | | GCTGTACAATAAGCTGCTGATATTCCATGCTGGACAGAATTTTCC |
| | | CAGTATTTTTTATAGAGTGCCAGGCTTTTCCTAGACTTCATGTCAT |
| | | ACAATACTTAACTTGTTTGGAGTGGGTGGAGATGGAAACATAGTC |
| | | TATTGAAAACATCACTGCTTCCTCCCTGAAGTTTAAAGAGCCTAT |
| | | TTTTATCCTTTTAGATTCTATCTCTCAGGCAAAATCTCATAAAGAT |
| | | AAGTGGGGAGGAAAAAAAGGGGGTTATAATACCTAGGGAGTTTG |
| | | CTTTTGCTAATTGAATACTGTGCTCCTAGACTTCTATAAATACCAT |
| | | TACAAATGGGTCCCAGCTTGTGGTAATACTCACCCTCCTCATTGA |
| | | GTCTTCTGTCCCATGGCACAGCCTTTCCCTCCAAACTAGCATCTAC |
| | | CCCCATCTGGAAGCATGGGCAGCTCATGATATTATCAACTATTGC |
| | | TATTGGAAAGTGATTTGGACTTGAAAGCACTAGATATTTTTTACC |
| | | TCTTGGGGAGGCAGTTTAGCAGAGTGGTTAACTGGTGAGCTCCAG |
| | | AATCAGAAGGAATAGGTCCAAATTCCAACCACTATTACATCTCCA |
| | | TCATAAGAAATTAGGCAAGTTGTTTATCCTAAGTTTCAGATTCCTT |
| | | AAAGATAAAACAGTCAAGACAGTAGTACTTATCCCTGAGAGAAG |
| | | TATAGGAAACAAGAAAATATATGCAATTTACATACATACTACAAT |
| | | CCCCAGCACATGACAAATGTTCAAGTAATGGGAACTGTTATTATT |
| | | TTAGCCCTTTGTCTATCAGTTTGTTCCTCTGTGACCTCAAGCACAT |
| | | TACTAAATGTTAGCGAGCTTCAGCTTGTACGTGGGACTGACAGGA |
| | | ATAACACCGCATCACCTCATGTGGTGATTGTAAGGATTCAGTGAT |
| | | ATTATTTTGTAAACTGTAAAGCCTTTGCAAATGTTAAGCAAGATT |
| | | ATTATTATTGCCGTTGTTATTAGTCCTCAGTGATCTTTTTTTTTTTT |
| | | TTTTTTTTTTTTTTTTTTTTTTTTTTTGGAGACAGAGTTTTACTCTGT |
| | | CGCCAAGGCTGGAATGCAGTGGCACAATCTCAGCTCACTGCAACC |
| | | TCCGCCTCCTGGGTTCAAGCAATTTTCCTGCCTCAGCCTCCTGAGT |
| | | AGCTGAAACTACAGGCACACGCCACCACACCAGGCTAATTTTTTG |
| | | TATTTTTAGTAGAGACGGGGTTTCACCATGTTGGCCAGGCTGGTC |
| | | TCCAGCTCCTGACCTCAAGTGATCTGCCCACCTCGGCCTCCCAAA |
| | | GTGCTGGGATTACAGGTGTGAGCCACCACACCTGGCACAGTAATC |
| | | TTAATTGAAAAGTCTGTGGATAGCTTTCCAAAGGAAAGCTTGGAG |
| | | CTTGGATAAGAACCAAGAGATAATGGGAGAAGGTGAATGGCCTC |
| | | TTCAGGGCCTTTTCTAGCACCCTAAATATGCGTGTCTGTCCATAAT |
| | | GGGTAATCATATATATCACAAATCAAACCCTCCACAAACTTATTT |
| | | CCTAATGTGTTTGTTAACCTTTCCTTCTAAAGGGTAAACTTCTTTA |
| | | ACCAACCCCAGTGAGCTGGAGGATCAATGTTTTCTTAATAGTCTT |
| | | ACCTTCGTTGGTGTCAATAGGAAACAGTATTTACTCACTACTGTTT |
| | | TCCTTTTAAAAATCTGTCTAGTTGCATACTAGAAACAGTTTCAGCT |
| | | GGTTTGTTTGTATTGGACAAGCTGCTGAAGTGAAAAGTTTTTGCTT |
| | | GACTGAATGTGAGACAGTTTCATAACTCTTCAAGAAGTGCACCAA |
| | | AGGTGGGTGCCAGCTCTGATGACGGCTGCTTCTAACATGCCTCCA |
| | | CTTGCCGCCCATTGTCAAGGGTGGCTGGCGTAATTAAGTTAAGAC |
| | | AATGAGCAAAGCAACAGATGCAACTGAGACCTAGTCCCTGAGTG |
| | | CTTTTGTTTTGTCACTGTCATTGTCTGCAACAAAGAAGTCACATGT |
| | | GACAGCCTGGGAAGAGAGCCAAATGCAAACCAGACGATATCCCA |
| | | GCTGGTTTGAATGGCCTCCACCGTGCACGTGTGTGCATGGGAATC |
| | | ATGCTACTTGGTACAGCATCTGCTTCACTCAAGTGAGTTTCAGCC |
| | | CATGGCTTTGCTGTGATGCTGAGACAGACCCAGAAGAAACAGAC |
| | | CAGGGAATCCCTCCGCTCAGACTTTACACTTTATACCTTGTGCTTT |
| | | GAGAGAAAAGAAAAAGAATCTCTCTATTGGAGACAAAAAATAGG |
| | | ATGTATGTGGTTGGTCAATCTAACCTCAATTCTTTTTGCTATAGCC |
| | | CCCCGCTAATTTAAAGAGTGAAGCATAGATGGTATCTTAATGTTT |
| | | TCTTGTAGAAATTTGGGATTAATTTGGCTTGAGAGGAAGAATGGA |
| | | GATTAAACGCTTTATGAGGCTTTCTTTTAATTTGTTCCCATTTCAT |
| | | TCCTGAATATTTTCTTAGTTTGGGCATTGCAGATGTTTAAAGAACT |
| | | TCTTATTTTGAGCTGGTATGCCTCTTAAACAGAAAAACAAAAGGT |
| | | AAAATTCAAATTAGTGTGTTTCTCCGCCTGTTAATTAATTTGGTTA |
| | | GTAGTTAGGCAGAGAGATGGCATCCTTAATAATATCTATTTTGCG |
| | | GGTTTGATCAGCTACAGACCATCAACAGTGTTGATTGAGAATTGA |
| | | ACAAAAACATTTCAAGGAGTTTGGGAACATTAGGGATGCTATTCT |
| | | GTGGCCCCATGTGTCCTTCTCTCATTTTTCTAGAGAACTCCTATAA |
| | | GAAAGCAGAACACGGCCAGGCATGATGGCTCATGCCTGTAATCC |
| | | CAGCACTTCAGGAGGCTGAGGCAGGCAGATCACCTGAGGTCAGG |
| | | AGTTCAAGACCAGCCTGGCCAACATGGTGAAACCCTATCTCTATT |
| | | AAAAATACAAAAAATTAGCTGGGCATGATGGCGCGTGCCTGTAA |
| | | TCCCAGCTACTTGGGAGGCTGAGGCAGGAGAATCACTTGAACTG |
| | | GGAGGCAAAGGTTGCAGTGAGCCTAGATCACACCACTGCACTCC |
| | | AGCCTGGGTGACAGAGTGAGACTCCAACTCAAAAAAAAGAAAGA |
| | | AAGAAAGAAAGAAAGCAGAACCCAATGGAAGATTAAGAACACA |
| | | CATTTAGCTTACGCCTGTAATACCAGCACTTTGGGAGGCCAAGGC |
| | | GGGTGGATCACAAGGTCAGAAGTTCGAGACCAACCTGGCCAATA |
| | | TGGTGAAACCCCATCTCTACTAAAAAGTACAAAAATTAGCCATGC |
| | | ATGGTGGCAGGCGTCTGTAATCCCAGCTACTACAGAGGCTGAGGC |
| | | AGGAGAATCACTTGAACCCGGGAGGCAGAGGTTGCAGTGAGCTG |
| | | AGAACGCGCCACTGCACTCCAGCCTGGGTGACAGAGCGAGACTC |
| | | CATCTCAAAAAAAAAAAACAACAAAAAAAAACAAAACACAAGTT |
| | | TACTGGGAACTTAGCAGTAGATGCTTTGCACCACAACAAATGTAT |
| | | CTTAAGTGGTCTTTTGTGATATTTGAGGGAAAGTGCCAGAATTTA |
| | | AAACAAATGGCATTTCAAGTTATTCTATACAAATGCCCAGTTTCT |
| | | TTCTACCATCTTTTTTTCCTTTTTGCAGTGGTCACTGAGCTATTTTA |
| | | GTGAATGTTTTTACACAATGATGCCATCTTCCTTCTACTCAGTCAG |
| | | TACAAGATGTTGACCATCGACTCATAAAACACTAGCTACCTTTCA |
| | | TGAAGGACTTGGTGATAACTCTCATGTTCCAAGTAGAACCGGAAA |
| | | ACATGTGTAAGAAAACCTGCCGATCCCTATGGGCCTTGGCCAATA |
| | | GGTATTATTCCCAAGGGGTGGCAGTTTATCTTTTTCCCCAGCCTTC |
| | | ATATTAAAACCTCTCACCTTCTCCAGGTCTCAGGTCTGTGTAATCT |
| | | CAAATGTGCTTTAGCTCCTCACAATATTGTAACTGTGTGGGTGTTC |
| | | ATTACCTTAGCCAGAAGACAGTTTACAGATTCCAGGTCTCATGGA |
| | | GAGAACTTTTGTTTTTGGTTATGAACCTCACTGTATACCAATAATT |
| | | ATCCATTACATCCTTCTGTAGAGGGCTCTCTGGCTAGAGATAAAA |
| | | CCAAAAAAAGAAGTACCTCAGGTTTATGCATATAAATGCCAGTTC |
| | | CTCCTTGATTTTATTTCAAAACTCCTGTCTACATACTTTGCAATTT |
| | | AAATACATTCAAGGATAAAGTAATAACTGTAGGAAAAGTATTAT |
| | | AATATAATGACTTAGTTCTGCACATCACAAGGGGGTCCCTCATAC |
| | | TCATTCATTCATTTCACTCATTTTACAGATATTTATTGAGCACCTG |
| | | CAATAACCTGCACACTGCTCTAGACACTGGGACTATAACAGTAAA |
| | | CAGACAGATACATCTCTGGTCTCACAGGGCTTCTATTCTAAGCAA |
| | | AACTCAATATCCAGGCCGGGTGCAGTGGCTCATGCCTGGAATGCC |
| | | AGCACTTTGGGAGACCAAGGCCAGGCAGATCACCTGAGCCCACT |
| | | AGTTGAAGACCAGCCTGGGCAATATAGCAAAACCCCGTCTCTACA |
| | | AAAAAAAAAAAAAAAAAAAAAAAAAAATTGTCAAGGCATGGTG |
| | | GCATGCGCCTGTGGTCCCAGCTACTTAGGAGGCTGAGGCAGGAG |
| | | GATTGTGTAAGCCTGGGAGGCAGAGGTTGCAGTGACCTGAGATG |
| | | GCACCACCACACTCCAGCCTGGGCAACAGAGTGAGACCCTGTCC |
| | | AAAAAAAAAAAACCCTCACTATCCTTAAGATAACATCATTGCTTG |
| | | TTGATGAGTGAATGTTAACACCAAATTAGGAACCCAGGACTTTTA |
| | | GTCTTGGCATGGTTACTTTCCAATAAAGATGACAATACTAAGAAG |
| | | AGAAAAATGATTTAATAATGATAATAGTGGCTAATACTTATGTAG |
| | | TGCTTACCATGTGCCAGGTCTATTGTAAGTACTTTTATATATATTA |
| | | ATTATTTAATCTTTGATCCTATAAGGTAGATATTATTGTTACCCTA |
| | | GTTTATAGATGAAGAAACGGAAACACAAGAGATTGCCACTCATA |
| | | CAAGTTTACACAGCCAGAAAATAGAAAAGCTACGAGTTGAGCTC |
| | | AGCCCAGTATGTCTATGATTTTACAGACTCAAAATTAATTATAAG |
| | | ATTTCCTAATCTTCGATTTCTGAAACTCTGCCTTGCTCTAGAGGAA |
| | | AACAAGAAAAACAATGAAAAATAAATGTCTCTTTTTTACAAAAAT |
| | | TAAAACAGAACAAACTGCAATAAAACAACAGAGGATGAATCCAG |
| | | AATGTGATTGATTTTTTTTCTTACTAGGAAAGGATCTAGAGGCCA |
| | | GAAGGCTGGATTTTTCAGGATCTCCTTTCAATCAATGAATCTGTG |
| | | ATAGAAGCAGATGAATCAAATCTCATCTTTGTGTGATTATAAAGC |
| | | TGTCTGTGGTATTCACGCCACCAGGGGTACATAGAAGATGCCTGA |
| | | GTGAGGTTTGGCAAAAGTACTAAGGGCCTGTCCACCTATACATGC |
| | | CCTTCTCAGGAAAACCAAGGTTCAAGCTCTCTATTAGCTCAACTG |
| | | GTAAGGCGTAAGACATGGAAGGTTGAGGCCCAATGTTAGAAATA |
| | | GATGGATACATAAAACTTCATCAAGTTAATGTCACTTTTTCTCCTT |
| | | TATTTATAG |
| SEQ ID | IGF-1 (exon | GAAGTACATTTGAAGAACGCAAGTAGAGGGAGTGCAGGAAACAA |
| NO. 5 | 6-2) | GAACTACAGGATGTAG |
| SEQ ID | IGF-1 | GTAAGCCCACCTGGGTGGGATCCAGCCATCCTCAAGTGGTCTCTC |
| NO. 6 | (intron 4 | TCTTGTGCATGTGGGTGGGCCAAGCAGAAATCCTGCCCCATAGTC |
| | used in IGF- | TCCTGGCTTACAAGTCAGAAAAGCTCCTTTGCACCAAAGGGATGG |
| | 1X6) | ATTACATCCCCATCTCTTTGGTCACTCTGCATTGCAAATTTCCCCT |
| | | CCCACCGCTATGGACGATGTGATGATTGGAAGATGTTACAAAACA |
| | | GTGGCTAAACAAACATGGGCTTTGGTGTCAGACAAAAGTGAAGT |
| | | CCTGGCTTTCTCACACACCAGCTTAGAGAGAAAAGACTTTTAGGT |
| | | GAATGTGGCAGGAAAGCGTGCTTGCTGGGGCAAAGGCAGATTCA |
| | | TTCTTTCTCTTCCCAG |
| SEQ ID | IGF-1 | GTAAGCCCACCTGGGTGGGATCCAGCCATCCTCAAGTGGTCTCTC |
| NO. 7 | (intron 4 | TCTTGTGCATGTGGGTGGGCCAAGCAGAAATCCTGCCCCATAGTC |
| | used in IGF- | TCCTGGCTTACAAGTCAGAAAAGCTCCTTTGCACCAAAGGGATGG |
| | 1X10) | ATTACATCCCCATCTCTTTGCTAAACAAACATGGGCTTTGGTGTCA |
| | | GACAAAAGTGAAGTCCTGGCTTTCTCACACACCAGCTTAGAGAGA |
| | | AAAGACTTTTAGGTGAATGTGGCAGGAAAGCGTGCTTGCTGGGG |
| | | CAAAGGCAGATTCATTCTTTCTCTTCCCAG |
| SEQ ID | IGF-1 | CTTTTTCTCCTTTATTTATAG |
| NO.8 | (intron 5 | |
| | used in IGF- | |
| | 1X6 and | |
| | IGF-1X10) | |
| SEQ ID | IGF-1X6 | ATGGGAAAAATCAGCAGTCTTCCAACCCAATTATTTAAGTGCTGC |
| NO. 9 | | TTTTGTGATTTCTTGAAGGTGAAGATGCACACCATGTCCTCCTCGC |
| | | ATCTCTTCTACCTGGCGCTGTGCCTGCTCACCTTCACCAGCTCTGC |
| | | CACGGCTGGACCGGAGACGCTCTGCGGGGCTGAGCTGGTGGATG |
| | | CTCTTCAGTTCGTGTGTGGAGACAGGGGCTTTTATTTCAACAAGC |
| | | CCACAGGGTATGGCTCCAGCAGTCGGAGGGCGCCTCAGACAGGC |
| | | ATCGTGGATGAGTGCTGCTTCCGGAGCTGTGATCTAAGGAGGCTG |
| | | GAGATGTATTGCGCACCCCTCAAGCCTGCCAAGTCAGCTCGCTCT |
| | | GTCCGTGCCCAGCGCCACACCGACATGCCCAAGACCCAGAAGGT |
| | | AAGCCCACCTGGGTGGGATCCAGCCATCCTCAAGTGGTCTCTCTC |
| | | TTGTGCATGTGGGTGGGCCAAGCAGAAATCCTGCCCCATAGTCTC |
| | | CTGGCTTACAAGTCAGAAAAGCTCCTTTGCACCAAAGGGATGGAT |
| | | TACATCCCCATCTCTTTGGTCACTCTGCATTGCAAATTTCCCCTCC |
| | | CACCGCTATGGACGATGTGATGATTGGAAGATGTTACAAAACAGT |
| | | GGCTAAACAAACATGGGCTTTGGTGTCAGACAAAAGTGAAGTCC |
| | | TGGCTTTCTCACACACCAGCTTAGAGAGAAAAGACTTTTAGGTGA |
| | | ATGTGGCAGGAAAGCGTGCTTGCTGGGGCAAAGGCAGATTCATT |
| | | CTTTCTCTTCCCAGTATCAGCCCCCATCTACCAACAAGAACACGA |
| | | AGTCTCAGAGAAGGAAAGGAAGTACATTTGAAGAACGCAAGTAG |
| | | CTTTTTCTCCTTTATTTATAGGAAGTACATTTGAAGAACGCAAGTA |
| | | GAGGGAGTGCAGGAAACAAGAACTACAGGATGTAG |
| SEQ ID | IGF-1X10 | ATGGGAAAAATCAGCAGTCTTCCAACCCAATTATTTAAGTGCTGC |
| NO. 10 | | TTTTGTGATTTCTTGAAGGTGAAGATGCACACCATGTCCTCCTCGC |
| | | ATCTCTTCTACCTGGCGCTGTGCCTGCTCACCTTCACCAGCTCTGC |
| | | CACGGCTGGACCGGAGACGCTCTGCGGGGCTGAGCTGGTGGATG |
| | | CTCTTCAGTTCGTGTGTGGAGACAGGGGCTTTTATTTCAACAAGC |
| | | CCACAGGGTATGGCTCCAGCAGTCGGAGGGCGCCTCAGACAGGC |
| | | ATCGTGGATGAGTGCTGCTTCCGGAGCTGTGATCTAAGGAGGCTG |
| | | GAGATGTATTGCGCACCCCTCAAGCCTGCCAAGTCAGCTCGCTCT |
| | | GTCCGTGCCCAGCGCCACACCGACATGCCCAAGACCCAGAAGGT |
| | | AAGCCCACCTGGGTGGGATCCAGCCATCCTCAAGTGGTCTCTCTC |
| | | TTGTGCATGTGGGTGGGCCAAGCAGAAATCCTGCCCCATAGTCTC |
| | | CTGGCTTACAAGTCAGAAAAGCTCCTTTGCACCAAAGGGATGGAT |
| | | TACATCCCCATCTCTTTGCTAAACAAACATGGGCTTTGGTGTCAG |
| | | ACAAAAGTGAAGTCCTGGCTTTCTCACACACCAGCTTAGAGAGAA |
| | | AAGACTTTTAGGTGAATGTGGCAGGAAAGCGTGCTTGCTGGGGC |
| | | AAAGGCAGATTCATTCTTTCTCTTCCCAGTATCAGCCCCCATCTAC |
| | | CAACAAGAACACGAAGTCTCAGAGAAGGAAAGGAAGTACATTTG |
| | | AAGAACGCAAGTAGCTTTTTCTCCTTTATTTATAGGAAGTACATTT |
| | | GAAGAACGCAAGTAGAGGGAGTGCAGGAAACAAGAACTACAGG |
| | | ATGTAG |
| SEQ ID | flHGF | MWVTKLLPALLLQHVLLHLLLLPIAIPYAEGQRKRRNTIHEFKKSAK |
| NO. 11 | | TTLIKIDPALKIKTKKVNTADQCANRCTRNKGLPFTCKAFVFDKARK |
| | | QCLWFPFNSMSSGVKKEFGHEFDLYENKDYIRNCIIGKGRSYKGTVS |
| | | ITKSGIKCQPWSSMIPHEHSFLPSSYRGKDLQENYCRNPRGEEGGPW |
| | | CFTSNPEVRYEVCDIPQCSEVECMTCNGESYRGLMDHTESGKICQR |
| | | WDHQTPHRHKFLPERYPDKGFDDNYCRNPDGQPRPWCYTLDPHTR |
| | | WEYCAIKTCADNTMNDTDVPLETTECIQGQGEGYRGTVNTIWNGIP |
| | | CQRWDSQYPHEHDMTPENFKCKDLRENYCRNPDGSESPWCFTTDPN |
| | | IRVGYCSQIPNCDMSHGQDCYRGNGKNYMGNLSQTRSGLTCSMWD |
| | | KNMEDLHRHIFWEPDASKLNENYCRNPDDDAHGPWCYTGNPLIPW |
| | | DYCPISRCEGDTTPTIVNLDHPVISCAKTKQLRVVNGIPTRTNIGWMV |
| | | SLRYRNKHICGGSLIKESWVLTARQCFPSRDLKDYEAWLGIHDVHG |
| | | RGDEKCKQVLNVSQLVYGPEGSDLVLMKLARPAVLDDFVSTIDLPN |
| | | YGCTIPEKTSCSVYGWGYTGLINYDGLLRVAHLYIMGNEKCSQHHR |
| | | GKVTLNESEICAGAEKIGSGPCEGDYGGPLVCEQHKMRMVLGVIVP |
| | | GRGCAIPNRPGIFVRVAYYAKWIHKII LTYKVPQS |
| SEQ ID | dHGF | MWVTKLLPALLLQHVLLHLLLLPIAIPYAEGQRKRRNTIHEFKKSAK |
| NO. 12 | | TTLIKIDPALKIKTKKVNTADQCANRCTRNKGLPFTCKAFVFDKARK |
| | | QCLWFPFNSMSSGVKKEFGHEFDLYENKDYIRNCIIGKGRSYKGTVS |
| | | ITKSGIKCQPWSSMIPHEHSYRGKDLQENYCRNPRGEEGGPWCFTSN |
| | | PEVRYEVCDIPQCSEVECMTCNGESYRGLMDHTESGKICQRWDHQT |
| | | PHRHKFLPERYPDKGFDDNYCRNPDGQPRPWCYTLDPHTRWEYCAI |
| | | KTCADNTMNDTDVPLETTECIQGQGEGYRGTVNTIWNGIPCQRWDS |
| | | QYPHEHDMTPENFKCKDLRENYCRNPDGSESPWCFTTDPNIRVGYC |
| | | SQIPNCDMSHGQDCYRGNGKNYMGNLSQTRSGLTCSMWDKNMED |
| | | LHRHIFWEPDASKLNENYCRNPDDDAHGPWCYTGNPLIPWDYCPIS |
| | | RCEGDTTPTIVNLDHPVISCAKTKQLRVVNGIPTRTNIGWMVSLRYR |
| | | NKHICGGSLIKESWVLTARQCFPSRDLKDYEAWLGIHDVHGRGDEK |
| | | CKQVLNVSQLVYGPEGSDLVLMKLARPAVLDDFVSTIDLPNYGCTIP |
| | | EKTSCSVYGWGYTGLINYDGLLRVAHLYIMGNEKCSQHHRGKVTL |
| | | NESEICAGAEKIGSGPCEGDYGGPLVCEQHKMRMVLGVIVPGRGCAI |
| | | PNRPGIFVRVAYYAKWIHKIILTYKVPQS |
| SEQ ID | HGF-X7 | ATGTGGGTGACCAAACTCCTGCCAGCCCTGCTGCTGCAGCATGTC |
| NO. 13 | (HGF | CTCCTGCATCTCCTCCTGCTCCCCATCGCCATCCCCTATGCAGAGG |
| | coding | GACAAAGGAAAAGAAGAAATACAATTCATGAATTCAAAAAATCA |
| | sequence in | GCAAAGACTACCCTAATCAAAATAGATCCAGCACTGAAGATAAA |
| | VM202) | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGGTAAGAACAG |
| | | TATGAAGAAAAGAGATGAAGCCTCTGTCTTTTTTACATGTTAACA |
| | | GTCTCATATTAGTCCTTCAGAATAATTCTACAATCCTAAAATAACT |
| | | TAGCCAACTTGCTGAATTGTATTACGGCAAGGTTTATATGAATTC |
| | | ATGACTGATATTTAGCAAATGATTAATTAATATGTTAATAAAATG |
| | | TAGCCAAAACAATATCTTACCTTAATGCCTCAATTTGTAGATCTC |
| | | GGTATTTGTGGATCCTGGGTAGGAAACACATTTGAATGGTATTTA |
| | | CTAAGATACTAAAATCCTTGGACTTCACTCTAATTTTAGTGCCATT |
| | | TAGAACTCAAGGTCTCAGTAAAAGTAGAAATAAAGCCTGTTAAC |
| | | AAAACACAAACTGAATATTAAAAATGTAACTGGATTTTCAAAGA |
| | | AATGTTTACTGGTATTACCTGTAGATGTATATTCTTTATTATGATC |
| | | TTTTGTGTAAAGTCTGGCAGACAAATGCAATATCTAATTGTTGAG |
| | | TCCAATATCACAAGCAGTACAAAAGTATAAAAAAGACTTGGCCTT |
| | | TTCTAATGTGTTAAAATACTTTATGCTGGTAATAACACTAAGAGT |
| | | AGGGCACTAGAAATTTTAAGTGAAGATAATGTGTTGCAGTTACTG |
| | | CACTCAATGGCTTACTATTATAAACCAAAACTGGGATCACTAAGC |
| | | TCCAGTCAGTCAAAATGATCAAAATTATTGAAGAGAATAAGCAA |
| | | TTCTGTTCTTTATTAGGACACAGTAGATACAGACTACAAAGTGGA |
| | | GTGTGCTTAATAAGAGGTAGCATTTGTTAAGTGTCAATTACTCTA |
| | | TTATCCCTTGGAGCTTCTCAAAATAACCATATAAGGTGTAAGATG |
| | | TTAAAGGTTATGGTTACACTCAGTGCACAGGTAAGCTAATAGGCT |
| | | GAGAGAAGCTAAATTACTTACTGGGGTCTCACAGTAAGAAAGTG |
| | | AGCTGAAGTTTCAGCCCAGATTTAACTGGATTCTGGGCTCTTTATT |
| | | CATGTTACTTCATGAATCTGTTTCTCAATTGTGCAGAAAAAAGGG |
| | | GGCTATTTATAAGAAAAGCAATAAACAAACAAGTAATGATCTCA |
| | | AATAAGTAATGCAAGAAATAGTGAGATTTCAAAATCAGTGGCAG |
| | | CGATTTCTCAGTTCTGTCCTAAGTGGCCTTGCTCAATCACCTGCTA |
| | | TCTTTTAGTGGAGCTTTGAAATTATGTTTCAGACAACTTCGATTCA |
| | | GTTCTAGAATGTTTGACTCAGCAAATTCACAGGCTCATCTTTCTAA |
| | | CTTGATGGTGAATATGGAAATTCAGCTAAATGGATGTTAATAAAA |
| | | TTCAAACGTTTTAAGGACAGATGGAAATGACAGAATTTTAAGGTA |
| | | AAATATATGAAGGAATATAAGATAAAGGATTTTTCTACCTTCAGC |
| | | AAAAACATACCCACTAATTAGTAAAATTAATAGGCGAAAAAAAG |
| | | TTGCATGCTCTTATACTGTAATGATTATCATTTTAAAACTAGCTTT |
| | | TTGCCTTCGAGCTATCGGGGTAAAGACCTACAGGAAAACTACTGT |
| | | CGAAATCCTCGAGGGGAAGAAGGGGGACCCTGGTGTTTCACAAG |
| | | CAATCCAGAGGTACGCTACGAAGTCTGTGACATTCCTCAGTGTTC |
| | | AGAAGTTGAATGCATGACCTGCAATGGGGAGAGTTATCGAGGTC |
| | | TCATGGATCATACAGAATCAGGCAAGATTTGTCAGCGCTGGGATC |
| | | ATCAGACACCACACCGGCACAAATTCTTGCCTGAAAGATATCCCG |
| | | ACAAGGGCTTTGATGATAATTATTGCCGCAATCCCGATGGCCAGC |
| | | CGAGGCCATGGTGCTATACTCTTGACCCTCACACCCGCTGGGAGT |
| | | ACTGTGCAATTAAAACATGCGCTGACAATACTATGAATGACACTG |
| | | ATGTTCCTTTGGAAACAACTGAATGCATCCAAGGTCAAGGAGAA |
| | | GGCTACAGGGGCACTGTCAATACCATTTGGAATGGAATTCCATGT |
| | | CAGCGTTGGGATTCTCAGTATCCTCACGAGCATGACATGACTCCT |
| | | GAAAATTTCAAGTGCAAGGACCTACGAGAAAATTACTGCCGAAA |
| | | TCCAGATGGGTCTGAATCACCCTGGTGTTTTACCACTGATCCAAA |
| | | CATCCGAGTTGGCTACTGCTCCCAAATTCCAAACTGTGATATGTC |
| | | ACATGGACAAGATTGTTATCGTGGGAATGGCAAAAATTATATGG |
| | | GCAACTTATCCCAAACAAGATCTGGACTAACATGTTCAATGTGGG |
| | | ACAAGAACATGGAAGACTTACATCGTCATATCTTCTGGGAACCAG |
| | | ATGCAAGTAAGCTGAATGAGAATTACTGCCGAAATCCAGATGAT |
| | | GATGCTCATGGACCCTGGTGCTACACGGGAAATCCACTCATTCCT |
| | | TGGGATTATTGCCCTATTTCTCGTTGTGAAGGTGATACCACACCTA |
| | | CAATAGTCAATTTAGACCATCCCGTAATATCTTGTGCCAAAACGA |
| | | AACAATTGCGAGTTGTAAATGGGATTCCAACACGAACAAACATA |
| | | GGATGGATGGTTAGTTTGAGATACAGAAATAAACATATCTGCGG |
| | | AGGATCATTGATAAAGGAGAGTTGGGTTCTTACTGCACGACAGTG |
| | | TTTCCCTTCTCGAGACTTGAAAGATTATGAAGCTTGGCTTGGAATT |
| | | CATGATGTCCACGGAAGAGGAGATGAGAAATGCAAACAGGTTCT |
| | | CAATGTTTCCCAGCTGGTATATGGCCCTGAAGGATCAGATCTGGT |
| | | TTTAATGAAGCTTGCCAGGCCTGCTGTCCTGGATGATTTTGTTAGT |
| | | ACGATTGATTTACCTAATTATGGATGCACAATTCCTGAAAAGACC |
| | | AGTTGCAGTGTTTATGGCTGGGGCTACACTGGATTGATCAACTAT |
| | | GATGGCCTATTACGAGTGGCACATCTCTATATAATGGGAAATGAG |
| | | AAATGCAGCCAGCATCATCGAGGGAAGGTGACTCTGAATGAGTC |
| | | TGAAATATGTGCTGGGGCTGAAAAGATTGGATCAGGACCATGTG |
| | | AGGGGGATTATGGTGGCCCACTTGTTTGTGAGCAACATAAAATGA |
| | | GAATGGTTCTTGGTGTCATTGTTCCTGGTCGTGGATGTGCCATTCC |
| | | AAATCGTCCTGGTATTTTTGTCCGAGTAGCATATTATGCAAAATG |
| | | GATACACAAAATTATTTTAACATATAAGGTACCACAGTCATAG |
| SEQ ID | aa sequence | MGKISSLPTQLFKCCFCDFLKVKMHTMSSSHLFYLALCLLTFTSSAT |
| NO. 14 | of Class I | AGPETLCGAELVDALQFVCGDRGFYFNKPTGYGSSSRRAPQTGIVDE |
| | IGF-1Ea | CCFRSCDLRRLEMYCAPLKPAKSARSVRAQRHTDMPKTQKEVHLK |
| | | NASRGSAGNKNYRM |
| SEQ ID | nucleotide | ATGGGAAAAATCAGCAGTCTTCCAACCCAATTATTTAAGTGCTGC |
| NO. 15 | sequence of | TTTTGTGATTTCTTGAAGGTGAAGATGCACACCATGTCCTCCTCGC |
| | Class I IGF- | ATCTCTTCTACCTGGCGCTGTGCCTGCTCACCTTCACCAGCTCTGC |
| | 1Ea | CACGGCTGGACCGGAGACGCTCTGCGGGGCTGAGCTGGTGGATG |
| | | CTCTTCAGTTCGTGTGTGGAGACAGGGGCTTTTATTTCAACAAGC |
| | | CCACAGGGTATGGCTCCAGCAGTCGGAGGGCGCCTCAGACAGGC |
| | | ATCGTGGATGAGTGCTGCTTCCGGAGCTGTGATCTAAGGAGGCTG |
| | | GAGATGTATTGCGCACCCCTCAAGCCTGCCAAGTCAGCTCGCTCT |
| | | GTCCGTGCCCAGCGCCACACCGACATGCCCAAGACCCAGAAGGA |
| | | AGTACATTTGAAGAACGCAAGTAGAGGGAGTGCAGGAAACAAGA |
| | | ACTACAGGATGTAG |
| SEQ ID | aa sequence | MGKISSLPTQLFKCCFCDFLKVKMHTMSSSHLFYLALCLLTFTSSAT |
| NO. 16 | of Class I | AGPETLCGAELVDALQFVCGDRGFYFNKPTGYGSSSRRAPQTGIVDE |
| | IGF-1 Ec | CCFRSCDLRRLEMYCAPLKPAKSARSVRAQRHTDMPKTQKYQPPST |
| | | NKNTKSQRRKGSTFEERK |
| SEQ ID | nucleotide | ATGGGAAAAATCAGCAGTCTTCCAACCCAATTATTTAAGTGCTGC |
| NO. 17 | sequence of | TTTTGTGATTTCTTGAAGGTGAAGATGCACACCATGTCCTCCTCGC |
| | Class I IGF- | ATCTCTTCTACCTGGCGCTGTGCCTGCTCACCTTCACCAGCTCTGC |
| | 1Ec | CACGGCTGGACCGGAGACGCTCTGCGGGGCTGAGCTGGTGGATG |
| | | CTCTTCAGTTCGTGTGTGGAGACAGGGGCTTTTATTTCAACAAGC |
| | | CCACAGGGTATGGCTCCAGCAGTCGGAGGGCGCCTCAGACAGGC |
| | | ATCGTGGATGAGTGCTGCTTCCGGAGCTGTGATCTAAGGAGGCTG |
| | | GAGATGTATTGCGCACCCCTCAAGCCTGCCAAGTCAGCTCGCTCT |
| | | GTCCGTGCCCAGCGCCACACCGACATGCCCAAGACCCAGAAGTA |
| | | TCAGCCCCCATCTACCAACAAGAACACGAAGTCTCAGAGAAGGA |
| | | AAGGAAGTACATTTGAAGAACGCAAGTAG |
| SEQ ID | aa sequence | MITPTVKMHTMSSSHLFYLALCLLTFTSSATAGPETLCGAELVDALQ |
| NO. 18 | of Class II | FVCGDRGFYFNKPTGYGSSSRRAPQTGIVDECCFRSCDLRRLEMYCA |
| | IGF-1 Ea | PLKPAKSARSVRAQRHTDMPKTQKEVHLKNASRGSAGNKNYRM |
| SEQ ID | nucleotide | ATGATTACACCTACAGTGAAGATGCACACCATGTCCTCCTCGCAT |
| NO. 19 | sequence of | CTCTTCTACCTGGCGCTGTGCCTGCTCACCTTCACCAGCTCTGCCA |
| | Class II | CGGCTGGACCGGAGACGCTCTGCGGGGCTGAGCTGGTGGATGCT |
| | IGF-1Ea | CTTCAGTTCGTGTGTGGAGACAGGGGCTTTTATTTCAACAAGCCC |
| | | ACAGGGTATGGCTCCAGCAGTCGGAGGGCGCCTCAGACAGGCAT |
| | | CGTGGATGAGTGCTGCTTCCGGAGCTGTGATCTAAGGAGGCTGGA |
| | | GATGTATTGCGCACCCCTCAAGCCTGCCAAGTCAGCTCGCTCTGT |
| | | CCGTGCCCAGCGCCACACCGACATGCCCAAGACCCAGAAGGAAG |
| | | TACATTTGAAGAACGCAAGTAGAGGGAGTGCAGGAAACAAGAAC |
| | | TACAGGATGTAG |
| SEQ ID | aa sequence | MGKISSLPTQLFKCCFCDFLKVKMHTMSSSHLFYLALCLLTFTSSAT |
| NO. 20 | of Class I | AGPETLCGAELVDALQFVCGDRGFYFNKPTGYGSSSRRAPQTGIVDE |
| | IGF-1 Eb | CCFRSCDLRRLEMYCAPLKPAKSARSVRAQRHTDMPKTQKYQPPST |
| | | NKNTKSQRRKGWPKTHPGGEQKEGTEASLQIRGKKKEQRREIGSRN |
| | | AECRGKKGK |
| SEQ ID | nucleotide | ATGGGAAAAATCAGCAGTCTTCCAACCCAATTATTTAAGTGCTGC |
| NO. 21 | sequence of | TTTTGTGATTTCTTGAAGGTGAAGATGCACACCATGTCCTCCTCGC |
| | Class I IGF- | ATCTCTTCTACCTGGCGCTGTGCCTGCTCACCTTCACCAGCTCTGC |
| | 1Eb | CACGGCTGGACCGGAGACGCTCTGCGGGGCTGAGCTGGTGGATG |
| | | CTCTTCAGTTCGTGTGTGGAGACAGGGGCTTTTATTTCAACAAGC |
| | | CCACAGGGTATGGCTCCAGCAGTCGGAGGGCGCCTCAGACAGGC |
| | | ATCGTGGATGAGTGCTGCTTCCGGAGCTGTGATCTAAGGAGGCTG |
| | | GAGATGTATTGCGCACCCCTCAAGCCTGCCAAGTCAGCTCGCTCT |
| | | GTCCGTGCCCAGCGCCACACCGACATGCCCAAGACCCAGAAGTA |
| | | TCAGCCCCCATCTACCAACAAGAACACGAAGTCTCAGAGAAGGA |
| | | AAGGTTGGCCAAAGACACATCCAGGAGGGGAACAGAAGGAGGG |
| | | GACAGAAGCAAGTCTGCAGATCAGAGGAAAGAAGAAAGAGCAG |
| | | AGGAGGGAGATTGGAAGTAGAAATGCTGAATGCAGAGGCAAAA |
| | | AAGGAAAATGA |
| SEQ ID | nucleotide | ATGTGGGTGA CCAAACTCCT GCCAGCCCTG CTGCTGCAGC |
| NO. 22 | sequence of | ATGTCCTCCT GCATCTCCTC CTGCTCCCCA TCGCCATCCC |
| | exons 1-4 of | CTATGCAGAG GGACAAAGGA AAAGAAGAAA TACAATTCAT |
| | human HGF | GAATTCAAAA AATCAGCAAA GACTACCCTA ATCAAAATAG |
| | | ATCCAGCACT GAAGATAAAA ACCAAAAAAG TGAATACTGC |
| | | AGACCAATGT GCTAATAGAT GTACTAGGAA TAAAGGACTT |
| | | CCATTCACTT GCAA GGCTTT TGTTTTTGAT AAAGCAAGAA |
| | | AACAATGCCT CTGGTTCCCC TTCAATAGCA TGTCAAGTGG |
| | | AGTGAAAAAA GAATTTGGCC ATGAATTTGA CCTCTATGAA |
| | | AACAAAGACT ACATTAGAAA CTGCATCATT GGTAAAGGAC |
| | | GCAGCTACAA GGGAACAGTA TCTATCACTA AGAGTGGCAT |
| | | CAAATGTCAG CCCTGGAGTT CCATGATACC ACACGAACAC AG |
| SEQ ID | Nucleotide | CC TACAGGAAAA CTACTGTCGA AATCCTCGAG GGGAAGAAGG |
| NO. 23 | sequence of | GGGACCCTGG TGTTTCACAA GCAATCCAGA GGTACGCTAC |
| | exon 5-18 of | GAAGTCTGTG ACATTCCTCA GTGTTCAGAA GTTGAATGCA |
| | human HGF | TGACCTGCAA TGGGGAGAGT TATCGAGGTC TCATGGATCA |
| | | TACAGAATCA GGCAAGATTT GTCAGCGCTG GGATCATCAG |
| | | ACACCACACC GGCACAAATT CTTGCCTGAA AGATATCCCG |
| | | ACAAGGGCTT TGATGATAAT TATTGCCGCA ATCCCGATGG |
| | | CCAGCCGAGG CCATGGTGCT ATACTCTTGA CCCTCACACC |
| | | CGCTGGGAGT ACTGTGCAAT TAAAACATGC GCTGACAATA |
| | | CTATGAATGA CACTGATGTT CCTTTGGAAA CAACTGAATG |
| | | CATCCAAGGT CAAGGAGAAG GCTACAGGGG CACTGTCAAT |
| | | ACCATTTGGA ATGGAATTCC ATGTCAGCGT TGGGATTCTC |
| | | AGTATCCTCA CGAGCATGAC ATGACTCCTG AAAATTTCAA |
| | | GTGCAAGGAC CTACGAGAAA ATTACTGCCG AAATCCAGAT |
| | | GGGTCTGAAT CACCCTGGTG TTTTACCACT GATCCAAACA |
| | | TCCGAGTTGG CTACTGCTCC CAAATTCCAA ACTGTGATAT |
| | | GTCACATGGA CAAGATTGTT ATCGTGGGAA TGGCAAAAAT |
| | | TATATGGGCA ACTTATCCCA AACAAGATCT GGACTAACAT |
| | | GTTCAATGTG GGACAAGAAC ATGGAAGACT TACATCGTCA |
| | | TATCTTCTGG GAACCAGATG CAAGTAAGCT GAATGAGAAT |
| | | TACTGCCGAA ATCCAGATGA TGATGCTCAT GGACCCTGGT |
| | | GCTACACGGG AAATCCACTC ATTCCTTGGG ATTATTGCCC |
| | | TATTTCTCGT TGTGAAGGTG ATACCACACC TACAATAGTC |
| | | AATTTAGACC ATCCCGTAAT ATCTTGTGCC AAAACGAAAC |
| | | AATTGCGAGT TGTAAATGGG ATTCCAACAC GAACAAACAT |
| | | AGGATGGATG GTTAGTTTGA GATACAGAAA TAAACATATC |
| | | TGCGGAGGAT CATTGATAAA GGAGAGTTGG GTTCTTACTG |
| | | CACGACAGTG TTTCCCTTCT CGAGACTTGA AAGATTATGA |
| | | AGCTTGGCTT GGAATTCATG ATGTCCACGG AAGAGGAGAT |
| | | GAGAAATGCA AACAGGTTCT CAATGTTTCC CAGCTGGTAT |
| | | ATGGCCCTGA AGGATCAGAT CTGGTTTTAA TGAAGCTTGC |
| | | CAGGCCTGCT GTCCTGGATG ATTTTGTTAG TACGATTGAT |
| | | TTACCTAATT ATGGATGCAC AATTCCTGAA AAGACCAGTT |
| | | GCAGTGTTTA TGGCTGGGGC TACACTGGAT TGATCAACTA |
| | | TGATGGCCTA TTACGAGTGG CACATCTCTA TATAATGGGA |
| | | AATGAGAAAT GCAGCCAGCA TCATCGAGGG AAGGTGACTC |
| | | TGAATGAGTC TGAAATATGT GCTGGGGCTG AAAAGATTGG |
| | | ATCAGGACCA TGTGAGGGGG ATTATGGTGG CCCACTTGTT |
| | | TGTGAGCAAC ATAAAATGAG AATGGTTCTT GGTGTCATTG |
| | | TTCCTGGTCG TGGATGTGCC ATTCCAAATC GTCCTGGTAT |
| | | TTTTGTCCGA GTAGCATATT ATGCAAAATG GATACACAAA |
| | | ATTATTTTAA CATATAAGGT ACCACAGTCA TAG |
| SEQ ID | Nucleotide | CGCGTTGACATTGATTATTGACTAGTTATTAATAGTAATCAATTAC |
| NO. 24 | sequence of | GGGGTCATTAGTTCATAGCCCATATATGGAGTTCCGCGTTACATA |
| | pCK vector | ACTTACGGTAAATGGCCCGCCTGGCTGACCGCCCAACGACCCCCG |
| | | CCCATTGACGTCAATAATGACGTATGTTCCCATAGTAACGCCAAT |
| | | AGGGACTTTCCATTGACGTCAATGGGTGGAGTATTTACGGTAAAC |
| | | TGCCCACTTGGCAGTACATCAAGTGTATCATATGCCAAGTCCGCC |
| | | CCCTATTGACGTCAATGACGGTAAATGGCCCGCCTGGCATTATGC |
| | | CCAGTACATGACCTTACGGGACTTTCCTACTTGGCAGTACATCTA |
| | | CGTATTAGTCATCGCTATTACCATGGTGATGCGGTTTTGGCAGTA |
| | | CACCAATGGGCGTGGATAGCGGTTTGACTCACGGGGATTTCCAAG |
| | | TCTCCACCCCATTGACGTCAATGGGAGTTTGTTTTGGCACCAAAA |
| | | TCAACGGGACTTTCCAAAATGTCGTAATAACCCCGCCCCGTTGAC |
| | | GCAAATGGGCGGTAGGCGTGTACGGTGGGAGGTCTATATAAGCA |
| | | GAGCTCGTTTAGTGAACCGTCAGATCGCCTGGAGACGCCATCCAC |
| | | GCTGTTTTGACCTCCATAGAAGACACCGGGACCGATCCAGCCTCC |
| | | GCGGCCGGGAACGGTGCATTGGAACGCGGATTCCCCGTGCCAAG |
| | | AGTGACGTAAGTACCGCCTATAGACTCTATAGGCACACCCCTTTG |
| | | GCTCTTATGCATGCTATACTGTTTTTGGCTTGGGGCCTATACACCC |
| | | CCGCTTCCTTATGCTATAGGTGATGGTATAGCTTAGCCTATAGGT |
| | | GTGGGTTATTGACCATTATTGACCACTCCCCTATTGGTGACGATA |
| | | CTTTCCATTACTAATCCATAACATGGCTCTTTGCCACAACTATCTC |
| | | TATTGGCTATATGCCAATACTCTGTCCTTCAGAGACTGACACGGA |
| | | CTCTGTATTTTTACAGGATGGGGTCCCATTTATTATTTACAAATTC |
| | | ACATATACAACAACGCCGTCCCCCGTGCCCGCAGTTTTTATTAAA |
| | | CATAGCGTGGGATCTCCACGCGAATCTCGGGTACGTGTTCCGGAC |
| | | ATGGGCTCTTCTCCGGTAGCGGCGGAGCTTCCACATCCGAGCCCT |
| | | GGTCCCATGCCTCCAGCGGCTCATGGTCGCTCGGCAGCTCCTTGC |
| | | TCCTAACAGTGGAGGCCAGACTTAGGCACAGCACAATGCCCACC |
| | | ACCACCAGTGTGCCGCACAAGGCCGTGGCGGTAGGGTATGTGTCT |
| | | GAAAATGAGCTCGGAGATTGGGCTCGCACCGCTGACGCAGATGG |
| | | AAGACTTAAGGCAGCGGCAGAAGAAGATGCAGGCAGCTGAGTTG |
| | | TTGTATTCTGATAAGAGTCAGAGGTAACTCCCGTTGCGGTGCTGT |
| | | TAACGGTGGAGGGCAGTGTAGTCTGAGCAGTACTCGTTGCTGCCG |
| | | CGCGCGCCACCAGACATAATAGCTGACAGACTAACAGACTGTTCC |
| | | TTTCCATGGGTCTTTTCTGCAGTCACCGTCCTTGACACGAAGCTTG |
| | | GTACCGAGCTCGGATCCACTAGTCCAGTGTGGTGGAATTCTGCAG |
| | | ATATCCAGCACAGTGGCGGCCGCTCGAGTCTAGAGGGCCCGTTTA |
| | | AACCCGCTGATCAGCCTCGACTGTGCCTTCTAGTTGCCAGCCATC |
| | | TGTTGTTTGCCCCTCCCCCGTGCCTTCCTTGACCCTGGAAGGTGCC |
| | | ACTCCCACTGTCCTTTCCTAATAAAATGAGGAAATTGCATCGCAT |
| | | TGTCTGAGTAGGTGTCATTCTATTCTGGGGGGTGGGGTGGGGCAG |
| | | GACAGCAAGGGGGAGGATTGGGAAGACAATAGCAGGCATGCTGG |
| | | GGAGTCGAAATTCAGAAGAACTCGTCAAGAAGGCGATAGAAGGC |
| | | GATGCGCTGCGAATCGGGAGCGGCGATACCGTAAAGCACGAGGA |
| | | AGCGGTCAGCCCATTCGCCGCCAAGCTCTTCAGCAATATCACGGG |
| | | TAGCCAACGCTATGTCCTGATAGCGGTCCGCCACACCCAGCCGGC |
| | | CACAGTCGATGAATCCAGAAAAGCGGCCATTTTCCACCATGATAT |
| | | TCGGCAAGCAGGCATCGCCATGGGTCACGACGAGATCCTCGCCGT |
| | | CGGGCATGCTCGCCTTGAGCCTGGCGAACAGTTCGGCTGGCGCGA |
| | | GCCCCTGATGCTCTTCGTCCAGATCATCCTGATCGACAAGACCGG |
| | | CTTCCATCCGAGTACGTGCTCGCTCGATGCGATGTTTCGCTTGGTG |
| | | GTCGAATGGGCAGGTAGCCGGATCAAGCGTATGCAGCCGCCGCA |
| | | TTGCATCAGCCATGATGGATACTTTCTCGGCAGGAGCAAGGTGAG |
| | | ATGACAGGAGATCCTGCCCCGGCACTTCGCCCAATAGCAGCCAGT |
| | | CCCTTCCCGCTTCAGTGACAACGTCGAGCACAGCTGCGCAAGGAA |
| | | CGCCCGTCGTGGCCAGCCACGATAGCCGCGCTGCCTCGTCTTGCA |
| | | GTTCATTCAGGGCACCGGACAGGTCGGTCTTGACAAAAAGAACC |
| | | GGGCGCCCCTGCGCTGACAGCCGGAACACGGCGGCATCAGAGCA |
| | | GCCGATTGTCTGTTGTGCCCAGTCATAGCCGAATAGCCTCTCCAC |
| | | CCAAGCGGCCGGAGAACCTGCGTGCAATCCATCTTGTTCAATCAT |
| | | GCGAAACGATCCTCATCCTGTCTCTTGATCAGATCTTGATCCCCTG |
| | | CGCCATCAGATCCTTGGCGGCAAGAAAGCCATCCAGTTTACTTTG |
| | | CAGGGCTTCCCAACCTTACCAGAGGGCGCCCCAGCTGGCAATTCC |
| | | GGTTCGCTTGCTGTCCATAAAACCGCCCAGTCTAGCTATCGCCAT |
| | | GTAAGCCCACTGCAAGCTACCTGCTTTCTCTTTGCGCTTGCGTTTT |
| | | CCCTTGTCCAGATAGCCCAGTAGCTGACATTCATCCGGGGTCAGC |
| | | ACCGTTTCTGCGGACTGGCTTTCTACGTGAAAAGGATCTAGGTGA |
| | | AGATCCTTTTTGATAATCTCATGACCAAAATCCCTTAACGTGAGTT |
| | | TTCGTTCCACTGAGCGTCAGACCCCGTAGAAAAGATCAAAGGATC |
| | | TTCTTGAGATCCTTTTTTTCTGCGCGTAATCTGCTGCTTGCAAACA |
| | | AAAAAACCACCGCTACCAGCGGTGGTTTGTTTGCCGGATCAAGAG |
| | | CTACCAACTCTTTTTCCGAAGGTAACTGGCTTCAGCAGAGCGCAG |
| | | ATACCAAATACTGTTCTTCTAGTGTAGCCGTAGTTAGGCCACCAC |
| | | TTCAAGAACTCTGTAGCACCGCCTACATACCTCGCTCTGCTAATC |
| | | CTGTTACCAGTGGCTGCTGCCAGTGGCGATAAGTCGTGTCTTACC |
| | | GGGTTGGACTCAAGACGATAGTTACCGGATAAGGCGCAGCGGTC |
| | | GGGCTGAACGGGGGGTTCGTGCACACAGCCCAGCTTGGAGCGAA |
| | | CGACCTACACCGAACTGAGATACCTACAGCGTGAGCTATGAGAA |
| | | AGCGCCACGCTTCCCGAAGGGAGAAAGGCGGACAGGTATCCGGT |
| | | AAGCGGCAGGGTCGGAACAGGAGAGCGCACGAGGGAGCTTCCAG |
| | | GGGGAAACGCCTGGTATCTTTATAGTCCTGTCGGGTTTCGCCACC |
| | | TCTGACTTGAGCGTCGATTTTTGTGATGCTCGTCAGGGGGGCGGA |
| | | GCCTATGGAAAAACGCCAGCAACGCGGCCTTTTTACGGTTCCTGG |
| | | CCTTTTGCTGGCCTTTTGCTCACATG |
| SEQ ID | Nucleotide | GTAAGAACAGTATGAAGAAAAGAGATGAAGCCTCTGTCTTTTTTA |
| NO. 25 | sequence of | CATGTTAACAGTCTCATATTAGTCCTTCAGAATAATTCTACAATCC |
| | intron 4 of | TAAAATAACTTAGCCAACTTGCTGAATTGTATTACGGCAAGGTTT |
| | human HGF | ATATGAATTCATGACTGATATTTAGCAAATGATTAATTAATATGT |
| | | TAATAAAATGTAGCCAAAACAATATCTTACCTTAATGCCTCAATT |
| | | TGTAGATCTCGGTATTTGTGAAATAATAACGTAAACTTCGTTTAA |
| | | AAGGATTCTTCTTCCTGTCTTTGAGAAAGTACGGCACTGTGCAGG |
| | | GGGAGAGGTTGATTGTGAAAAATCAGAGGTAGATGAGAATCTTA |
| | | CTGAGGGCTGAGGGTTCTTTAACCTTGGTGGATCTCAACATTGGT |
| | | TGCACATTAAAATCACCTGCTGCAAGCCCTTGACGAATCTTACTT |
| | | AGAAGATGACAACACAGAACAATTAAATCAGAATCTCTGGGGAG |
| | | AATAGGGCACCAGTATTTTTTGAGCTCCCACCATGATTCCAAAGT |
| | | GCAGCCAAATTTGAGAACCACTGCTAAAAGCTCAAGCTTCAGATT |
| | | GACCAGCTTTTCCATCTCACCTATCGCCTAAAGACCAAATTGGAT |
| | | AAATGTGTTCATTACGACAGATGGGTACTATTTAAAGATGAGTAA |
| | | ACACAATATACTTAGGCTCGTCAGACTGAGAGTTTTAATCATCAC |
| | | TGAGGAAAAACATAGATATCTAATACTGACTGGAGTATTAGTCAA |
| | | GGCTTATTTCACACACAATTTTATCAGAAACCAAAGTAGTTTAAA |
| | | ACAGCTCTCCCCTTATTAGTAATGCATTGGAGGGTTTACTTTACCA |
| | | TGTACCTTGCTGAGCACTGTACCTTGTTAATCTCATTTACTTGTAA |
| | | TGAGAACCACACAGCGGGTAGTTTTATTGGTTCTATTTTACCTAC |
| | | ATGACAAAACTGAAGCATAAAAACACTTAGTAAGTTTTCAGTGTC |
| | | ATGCACAACTAGGAAGTGACATGGCCAGAATATAAGCCCAGTCA |
| | | CCATCACTCTATAACCTGCGCTTTTAACAACTTCAGGGCATGACA |
| | | CATTTGGCCGGTCAGTAGAACCCATGCTGTGATTTGTTTTTGCAGT |
| | | GGTGGTGATGACTGCCTTGTTGAATCCACTTTTTATTCTATTCCAT |
| | | TTTGGGGACACAATTCTGCAAGATGATTCTTCATTAGGAAACAGA |
| | | GATGAGTTATTGACCAACACAGAAAGAAAAAGAGTTTGTTGCTCC |
| | | ACACTGGGATTAAACCTATGATCTTGGCCTAATTAACACTAGCTA |
| | | GTAAGTGTCCAAGCTGATCATCTCTACAACATTTCAATAACAGAA |
| | | AACAACAATTTTCAAAATTAGTTACTTACAATTATGTAGAAATGC |
| | | CTCTAAAACACAGTATTTTCCTTATATTACAAAAACAAAAATTAT |
| | | AATTGGTTTTGTCCTCTTTTGAGAGTTTGCATGGTGTTACTCCCTG |
| | | CATAGTGAAGAAAACATTTTATTTAAGTAGATGGATCTAAGTTTT |
| | | TCATGAACAAAGGAATGACATTTGAAATCAATCCTACCCTAGTCC |
| | | AGGAGAATGCATTAGATTAACCTAGTAGAGGTCTTATTTCACCCT |
| | | GAGTTTTCTATGATCGTGATTCTCTGCTGGAGGAGTAATTGTGAA |
| | | ATAGATCTCTCTGGGAACTGGCTTCCTAGTCCAATCAGCTCTTTTA |
| | | CCAATGAACACTTCCTTGTGATATAGATGTTTATGGCCGAGAGGA |
| | | TCCAGTATATTAATAAAATCCCTTTTTGTATTCAATGAGGGAAAC |
| | | ACATAATTTTCATCAATTAGCAGCTTATTGGAATATCTGCATGAT |
| | | GGTTTAACACTTTTAAGTGTTGACTAAAGATTAATTTTACAGAAA |
| | | ATAGAAAAAGAAATATGTTTCTGTCTGGAGGAATGATTTATTGTT |
| | | GACCCCTAAATTGAAATATTTTACTAGTGGCTTAATGGAAAGATG |
| | | ATGAAAGATGATGAAATTAATGTAGAAGCTTAACTAGAAAATCA |
| | | GGTGACCTGATATCTACATCTGTATCCTTCATTGGCCACCCAGCAT |
| | | TCATTAATGAATCAGATGATGGAATAGATCAAGTTTCCTAGGAAC |
| | | ACAGTGAATATTAAAAGAAAACAAAGGGAGCCTAGCACCTAGAA |
| | | GACCTAGTTTATATTTCAAAGTATATTTGGATGTAACCCAATTTTA |
| | | AACATTTCCTCACTTGTCTCTCTTAAAGCCTTGCCAACAGCAAGG |
| | | ACAGAGAACCAAAAATAGTGTATATATGAATAAATGCTTATTACA |
| | | GAATCTGCTGACTGGCACATGCTTTGTGTGTAATGGGTTCTCATA |
| | | AACACTTGTTGAATGAACACACATAAGTGAAAGAGCATGGCTAG |
| | | GCTTCATCCCTTGGTCAAATATGGGGTGCTAAAGAAAAGCAGGG |
| | | GAAATACATTGGGACACTAACAAAAAAAAACAGTTAATTTAGGT |
| | | AAAAGATAAAATACACCACAGAATGAAGAAAAGAGATGACCCA |
| | | GACTGCTCTTTAACCTTCATGTCCTAGAGAGGTTTTTGATATGAAT |
| | | TGCATTCAGAATTGTGGAAAGGAGCCCATCTTTTCTCTTCATTTTG |
| | | ATTTTATTAACTCCAATGGGGGAATTTTATTCGTGTTTTGGCCATA |
| | | TCTACTTTTGATTTCTACATTATTCTCTCTTCCTTTCTACCTGTATT |
| | | TGTCCTAATAAATTGTTGACTTATTAATTCACTACTTCCTCACAGC |
| | | TTTTTTTTGGCTTTACAAATCCACTGGAAAGGTATATGGGTGTATC |
| | | ACTTTGTGTATTTCGGTGTGCATGTGTAGAGGGGACAAAAATCCT |
| | | CTCTCAAACTATAAATATTGAGTATTTGTGTATTGAACATTTGCTA |
| | | TAACTACTAGGTTTCTTAAATAATCTTAATATATAAAATGATATA |
| | | GAAAAAGGGAAATTATAGTTCGTATTATTCATCTAAGTGAAGAGA |
| | | TTAAAACCCAGGGAGTAAATAAATTGTCTAAGGACTAAGGTTGTA |
| | | TACTATTTAGGTGATAGATATGGGGCAACCGTATGGGTTTTATGA |
| | | TTAACAAATAAACTTCTCACCACTCTACCATATCAACTTTTCCATA |
| | | AAAGAGAGCTATAGTATTCTTTGCTTAAATAAATTTGATTAGTGC |
| | | ATGACTTCTTGAAAACATATAAAGCAAAAGTCACATTTGATTCTA |
| | | TCAGAAAAGTGAGTAAGCCATGGCCCAAACAAAAGATGCATTAA |
| | | AATATTCTGGAATGATGGAGCTAAAAGTAAGAAAAATGACTTTTT |
| | | AAAAAAGTTTACTGTTAGGAATTGTGAAATTATGCTGAATTTTAG |
| | | TTGCATTATAATTTTTGTCAGTCATACGGTCTGACAACCTGTCTTA |
| | | TTTCTATTTCCCCATATGAGGAATGCTAGTTAAGTATGGATATTAA |
| | | CTATTACTACTTAGATGCATTGAAGTTGCATAATATGGATAATAC |
| | | TTCACTGGTTCCCTGAAAATGTTTAGTTAGTAATAAGTCTCTTACA |
| | | CTATTTGTTTTGTCCAATAATTTATATTTTCTGAAGACTTAACTCT |
| | | AGAATACACTCATGTCAAAATGAAAGAATTTCATTGCAAAATATT |
| | | GCTTGGTACATGACGCATACCTGTATTTGTTTTGTGTCACAACATG |
| | | AAAAATGATGGTTTATTAGAAGTTTCATTGGGTAGGAAACACATT |
| | | TGAATGGTATTTACTAAGATACTAAAATCCTTGGACTTCACTCTA |
| | | ATTTTAGTGCCATTTAGAACTCAAGGTCTCAGTAAAAGTAGAAAT |
| | | AAAGCCTGTTAACAAAACACAAACTGAATATTAAAAATGTAACT |
| | | GGATTTTCAAAGAAATGTTTACTGGTATTACCTGTAGATGTATATT |
| | | CTTTATTATGATCTTTTGTGTAAAGTCTGGCAGACAAATGCAATAT |
| | | CTAATTGTTGAGTCCAATATCACAAGCAGTACAAAAGTATAAAAA |
| | | AGACTTGGCCTTTTCTAATGTGTTAAAATACTTTATGCTGGTAATA |
| | | ACACTAAGAGTAGGGCACTAGAAATTTTAAGTGAAGATAATGTG |
| | | TTGCAGTTACTGCACTCAATGGCTTACTATTATAAACCAAAACTG |
| | | GGATCACTAAGCTCCAGTCAGTCAAAATGATCAAAATTATTGAAG |
| | | AGAATAAGCAATTCTGTTCTTTATTAGGACACAGTAGATACAGAC |
| | | TACAAAGTGGAGTGTGCTTAATAAGAGGTAGCATTTGTTAAGTGT |
| | | CAATTACTCTATTATCCCTTGGAGCTTCTCAAAATAACCATATAA |
| | | GGTGTAAGATGTTAAAGGTTATGGTTACACTCAGTGCACAGGTAA |
| | | GCTAATAGGCTGAGAGAAGCTAAATTACTTACTGGGGTCTCACAG |
| | | TAAGAAAGTGAGCTGAAGTTTCAGCCCAGATTTAACTGGATTCTG |
| | | GGCTCTTTATTCATGTTACTTCATGAATCTGTTTCTCAATTGTGCA |
| | | GAAAAAAGGGGGCTATTTATAAGAAAAGCAATAAACAAACAAGT |
| | | AATGATCTCAAATAAGTAATGCAAGAAATAGTGAGATTTCAAAA |
| | | TCAGTGGCAGCGATTTCTCAGTTCTGTCCTAAGTGGCCTTGCTCAA |
| | | TCACCTGCTATCTTTTAGTGGAGCTTTGAAATTATGTTTCAGACAA |
| | | CTTCGATTCAGTTCTAGAATGTTTGACTCAGCAAATTCACAGGCT |
| | | CATCTTTCTAACTTGATGGTGAATATGGAAATTCAGCTAAATGGA |
| | | TGTTAATAAAATTCAAACGTTTTAAGGACAGATGGAAATGACAG |
| | | AATTTTAAGGTAAAATATATGAAGGAATATAAGATAAAGGATTTT |
| | | TCTACCTTCAGCAAAAACATACCCACTAATTAGTAAAATTAATAG |
| | | GCGAAAAAAAGTTGCATGCTCTTATACTGTAATGATTATCATTTT |
| | | AAAACTAGCTTTTTGCCTTCGAGCTATCGGGGTAAAGA |
| SEQ ID | Nucleotide | ATGTGGGTGACCAAACTCCTGCCAGCCCTGCTGCTGCAGCATGTC |
| NO. 26 | sequence of | CTCCTGCATCTCCTCCTGCTCCCCATCGCCATCCCCTATGCAGAGG |
| | HGF-X1 | GACAAAGGAAAAGAAGAAATACAATTCATGAATTCAAAAAATCA |
| | | GCAAAGACTACCCTAATCAAAATAGATCCAGCACTGAAGATAAA |
| | | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGGTAAGAACAG |
| | | TATGAAGAAAAGAGATGAAGCCTCTGTCTTTTTTACATGTTAACA |
| | | GTCTCATATTAGTCCTTCAGAATAATTCTACAATCCTAAAATAACT |
| | | TAGCCAACTTGCTGAATTGTATTACGGCAAGGTTTATATGAATTC |
| | | ATGACTGATATTTAGCAAATGATTAATTAATATGTTAATAAAATG |
| | | TAGCCAAAACAATATCTTACCTTAATGCCTCAATTTGTAGATCTC |
| | | GGTATTTGTGAAATAATAACGTAAACTTCGTTTAAAAGGATTCTT |
| | | CTTCCTGTCTTTGAGAAAGTACGGCACTGTGCAGGGGGAGAGGTT |
| | | GATTGTGAAAAATCAGAGGTAGATGAGAATCTTACTGAGGGCTG |
| | | AGGGTTCTTTAACCTTGGTGGATCTCAACATTGGTTGCACATTAA |
| | | AATCACCTGCTGCAAGCCCTTGACGAATCTTACTTAGAAGATGAC |
| | | AACACAGAACAATTAAATCAGAATCTCTGGGGAGAATAGGGCAC |
| | | CAGTATTTTTTGAGCTCCCACCATGATTCCAAAGTGCAGCCAAAT |
| | | TTGAGAACCACTGCTAAAAGCTCAAGCTTCAGATTGACCAGCTTT |
| | | TCCATCTCACCTATCGCCTAAAGACCAAATTGGATAAATGTGTTC |
| | | ATTACGACAGATGGGTACTATTTAAAGATGAGTAAACACAATATA |
| | | CTTAGGCTCGTCAGACTGAGAGTTTTAATCATCACTGAGGAAAAA |
| | | CATAGATATCTAATACTGACTGGAGTATTAGTCAAGGCTTATTTC |
| | | ACACACAATTTTATCAGAAACCAAAGTAGTTTAAAACAGCTCTCC |
| | | CCTTATTAGTAATGCATTGGAGGGTTTACTTTACCATGTACCTTGC |
| | | TGAGCACTGTACCTTGTTAATCTCATTTACTTGTAATGAGAACCAC |
| | | ACAGCGGGTAGTTTTATTGGTTCTATTTTACCTACATGACAAAACT |
| | | GAAGCATAAAAACACTTAGTAAGTTTTCAGTGTCATGCACAACTA |
| | | GGAAGTGACATGGCCAGAATATAAGCCCAGTCACCATCACTCTAT |
| | | AACCTGCGCTTTTAACAACTTCAGGGCATGACACATTTGGCCGGT |
| | | CAGTAGAACCCATGCTGTGATTTGTTTTTGCAGTGGTGGTGATGA |
| | | CTGCCTTGTTGAATCCACTTTTTATTCTATTCCATTTTGGGGACAC |
| | | AATTCTGCAAGATGATTCTTCATTAGGAAACAGAGATGAGTTATT |
| | | GACCAACACAGAAAGAAAAAGAGTTTGTTGCTCCACACTGGGAT |
| | | TAAACCTATGATCTTGGCCTAATTAACACTAGCTAGTAAGTGTCC |
| | | AAGCTGATCATCTCTACAACATTTCAATAACAGAAAACAACAATT |
| | | TTCAAAATTAGTTACTTACAATTATGTAGAAATGCCTCTAAAACA |
| | | CAGTATTTTCCTTATATTACAAAAACAAAAATTATAATTGGTTTTG |
| | | TCCTCTTTTGAGAGTTTGCATGGTGTTACTCCCTGCATAGTGAAGA |
| | | AAACATTTTATTTAAGTAGATGGATCTAAGTTTTTCATGAACAAA |
| | | GGAATGACATTTGAAATCAATCCTACCCTAGTCCAGGAGAATGCA |
| | | TTAGATTAACCTAGTAGAGGTCTTATTTCACCCTGAGTTTTCTATG |
| | | ATCGTGATTCTCTGCTGGAGGAGTAATTGTGAAATAGATCTCTCT |
| | | GGGAACTGGCTTCCTAGTCCAATCAGCTCTTTTACCAATGAACAC |
| | | TTCCTTGTGATATAGATGTTTATGGCCGAGAGGATCCAGTATATT |
| | | AATAAAATCCCTTTTTGTATTCAATGAGGGAAACACATAATTTTC |
| | | ATCAATTAGCAGCTTATTGGAATATCTGCATGATGGTTTAACACT |
| | | TTTAAGTGTTGACTAAAGATTAATTTTACAGAAAATAGAAAAAGA |
| | | AATATGTTTCTGTCTGGAGGAATGATTTATTGTTGACCCCTAAATT |
| | | GAAATATTTTACTAGTGGCTTAATGGAAAGATGATGAAAGATGAT |
| | | GAAATTAATGTAGAAGCTTAACTAGAAAATCAGGTGACCTGATAT |
| | | CTACATCTGTATCCTTCATTGGCCACCCAGCATTCATTAATGAATC |
| | | AGATGATGGAATAGATCAAGTTTCCTAGGAACACAGTGAATATTA |
| | | AAAGAAAACAAAGGGAGCCTAGCACCTAGAAGACCTAGTTTATA |
| | | TTTCAAAGTATATTTGGATGTAACCCAATTTTAAACATTTCCTCAC |
| | | TTGTCTCTCTTAAAGCCTTGCCAACAGCAAGGACAGAGAACCAAA |
| | | AATAGTGTATATATGAATAAATGCTTATTACAGAATCTGCTGACT |
| | | GGCACATGCTTTGTGTGTAATGGGTTCTCATAAACACTTGTTGAA |
| | | TGAACACACATAAGTGAAAGAGCATGGCTAGGCTTCATCCCTTGG |
| | | TCAAATATGGGGTGCTAAAGAAAAGCAGGGGAAATACATTGGGA |
| | | CACTAACAAAAAAAAACAGTTAATTTAGGTAAAAGATAAAATAC |
| | | ACCACAGAATGAAGAAAAGAGATGACCCAGACTGCTCTTTAACC |
| | | TTCATGTCCTAGAGAGGTTTTTGATATGAATTGCATTCAGAATTGT |
| | | GGAAAGGAGCCCATCTTTTCTCTTCATTTTGATTTTATTAACTCCA |
| | | ATGGGGGAATTTTATTCGTGTTTTGGCCATATCTACTTTTGATTTC |
| | | TACATTATTCTCTCTTCCTTTCTACCTGTATTTGTCCTAATAAATTG |
| | | TTGACTTATTAATTCACTACTTCCTCACAGCTTTTTTTTGGCTTTAC |
| | | AAATCCACTGGAAAGGTATATGGGTGTATCACTTTGTGTATTTCG |
| | | GTGTGCATGTGTAGAGGGGACAAAAATCCTCTCTCAAACTATAAA |
| | | TATTGAGTATTTGTGTATTGAACATTTGCTATAACTACTAGGTTTC |
| | | TTAAATAATCTTAATATATAAAATGATATAGAAAAAGGGAAATTA |
| | | TAGTTCGTATTATTCATCTAAGTGAAGAGATTAAAACCCAGGGAG |
| | | TAAATAAATTGTCTAAGGACTAAGGTTGTATACTATTTAGGTGAT |
| | | AGATATGGGGCAACCGTATGGGTTTTATGATTAACAAATAAACTT |
| | | CTCACCACTCTACCATATCAACTTTTCCATAAAAGAGAGCTATAG |
| | | TATTCTTTGCTTAAATAAATTTGATTAGTGCATGACTTCTTGAAAA |
| | | CATATAAAGCAAAAGTCACATTTGATTCTATCAGAAAAGTGAGTA |
| | | AGCCATGGCCCAAACAAAAGATGCATTAAAATATTCTGGAATGA |
| | | TGGAGCTAAAAGTAAGAAAAATGACTTTTTAAAAAAGTTTACTGT |
| | | TAGGAATTGTGAAATTATGCTGAATTTTAGTTGCATTATAATTTTT |
| | | GTCAGTCATACGGTCTGACAACCTGTCTTATTTCTATTTCCCCATA |
| | | TGAGGAATGCTAGTTAAGTATGGATATTAACTATTACTACTTAGA |
| | | TGCATTGAAGTTGCATAATATGGATAATACTTCACTGGTTCCCTG |
| | | AAAATGTTTAGTTAGTAATAAGTCTCTTACACTATTTGTTTTGTCC |
| | | AATAATTTATATTTTCTGAAGACTTAACTCTAGAATACACTCATGT |
| | | CAAAATGAAAGAATTTCATTGCAAAATATTGCTTGGTACATGACG |
| | | CATACCTGTATTTGTTTTGTGTCACAACATGAAAAATGATGGTTTA |
| | | TTAGAAGTTTCATTGGGTAGGAAACACATTTGAATGGTATTTACT |
| | | AAGATACTAAAATCCTTGGACTTCACTCTAATTTTAGTGCCATTTA |
| | | GAACTCAAGGTCTCAGTAAAAGTAGAAATAAAGCCTGTTAACAA |
| | | AACACAAACTGAATATTAAAAATGTAACTGGATTTTCAAAGAAAT |
| | | GTTTACTGGTATTACCTGTAGATGTATATTCTTTATTATGATCTTTT |
| | | GTGTAAAGTCTGGCAGACAAATGCAATATCTAATTGTTGAGTCCA |
| | | ATATCACAAGCAGTACAAAAGTATAAAAAAGACTTGGCCTTTTCT |
| | | AATGTGTTAAAATACTTTATGCTGGTAATAACACTAAGAGTAGGG |
| | | CACTAGAAATTTTAAGTGAAGATAATGTGTTGCAGTTACTGCACT |
| | | CAATGGCTTACTATTATAAACCAAAACTGGGATCACTAAGCTCCA |
| | | GTCAGTCAAAATGATCAAAATTATTGAAGAGAATAAGCAATTCTG |
| | | TTCTTTATTAGGACACAGTAGATACAGACTACAAAGTGGAGTGTG |
| | | CTTAATAAGAGGTAGCATTTGTTAAGTGTCAATTACTCTATTATCC |
| | | CTTGGAGCTTCTCAAAATAACCATATAAGGTGTAAGATGTTAAAG |
| | | GTTATGGTTACACTCAGTGCACAGGTAAGCTAATAGGCTGAGAGA |
| | | AGCTAAATTACTTACTGGGGTCTCACAGTAAGAAAGTGAGCTGAA |
| | | GTTTCAGCCCAGATTTAACTGGATTCTGGGCTCTTTATTCATGTTA |
| | | CTTCATGAATCTGTTTCTCAATTGTGCAGAAAAAAGGGGGCTATT |
| | | TATAAGAAAAGCAATAAACAAACAAGTAATGATCTCAAATAAGT |
| | | AATGCAAGAAATAGTGAGATTTCAAAATCAGTGGCAGCGATTTCT |
| | | CAGTTCTGTCCTAAGTGGCCTTGCTCAATCACCTGCTATCTTTTAG |
| | | TGGAGCTTTGAAATTATGTTTCAGACAACTTCGATTCAGTTCTAG |
| | | AATGTTTGACTCAGCAAATTCACAGGCTCATCTTTCTAACTTGATG |
| | | GTGAATATGGAAATTCAGCTAAATGGATGTTAATAAAATTCAAAC |
| | | GTTTTAAGGACAGATGGAAATGACAGAATTTTAAGGTAAAATAT |
| | | ATGAAGGAATATAAGATAAAGGATTTTTCTACCTTCAGCAAAAAC |
| | | ATACCCACTAATTAGTAAAATTAATAGGCGAAAAAAAGTTGCAT |
| | | GCTCTTATACTGTAATGATTATCATTTTAAAACTAGCTTTTTGCCT |
| | | TCGAGCTATCGGGGTAAAGACCTACAGGAAAACTACTGTCGAAA |
| | | TCCTCGAGGGGAAGAAGGGGGACCCTGGTGTTTCACAAGCAATC |
| | | CAGAGGTACGCTACGAAGTCTGTGACATTCCTCAGTGTTCAGAAG |
| | | TTGAATGCATGACCTGCAATGGGGAGAGTTATCGAGGTCTCATGG |
| | | ATCATACAGAATCAGGCAAGATTTGTCAGCGCTGGGATCATCAGA |
| | | CACCACACCGGCACAAATTCTTGCCTGAAAGATATCCCGACAAGG |
| | | GCTTTGATGATAATTATTGCCGCAATCCCGATGGCCAGCCGAGGC |
| | | CATGGTGCTATACTCTTGACCCTCACACCCGCTGGGAGTACTGTG |
| | | CAATTAAAACATGCGCTGACAATACTATGAATGACACTGATGTTC |
| | | CTTTGGAAACAACTGAATGCATCCAAGGTCAAGGAGAAGGCTAC |
| | | AGGGGCACTGTCAATACCATTTGGAATGGAATTCCATGTCAGCGT |
| | | TGGGATTCTCAGTATCCTCACGAGCATGACATGACTCCTGAAAAT |
| | | TTCAAGTGCAAGGACCTACGAGAAAATTACTGCCGAAATCCAGA |
| | | TGGGTCTGAATCACCCTGGTGTTTTACCACTGATCCAAACATCCG |
| | | AGTTGGCTACTGCTCCCAAATTCCAAACTGTGATATGTCACATGG |
| | | ACAAGATTGTTATCGTGGGAATGGCAAAAATTATATGGGCAACTT |
| | | ATCCCAAACAAGATCTGGACTAACATGTTCAATGTGGGACAAGA |
| | | ACATGGAAGACTTACATCGTCATATCTTCTGGGAACCAGATGCAA |
| | | GTAAGCTGAATGAGAATTACTGCCGAAATCCAGATGATGATGCTC |
| | | ATGGACCCTGGTGCTACACGGGAAATCCACTCATTCCTTGGGATT |
| | | ATTGCCCTATTTCTCGTTGTGAAGGTGATACCACACCTACAATAG |
| | | TCAATTTAGACCATCCCGTAATATCTTGTGCCAAAACGAAACAAT |
| | | TGCGAGTTGTAAATGGGATTCCAACACGAACAAACATAGGATGG |
| | | ATGGTTAGTTTGAGATACAGAAATAAACATATCTGCGGAGGATCA |
| | | TTGATAAAGGAGAGTTGGGTTCTTACTGCACGACAGTGTTTCCCT |
| | | TCTCGAGACTTGAAAGATTATGAAGCTTGGCTTGGAATTCATGAT |
| | | GTCCACGGAAGAGGAGATGAGAAATGCAAACAGGTTCTCAATGT |
| | | TTCCCAGCTGGTATATGGCCCTGAAGGATCAGATCTGGTTTTAAT |
| | | GAAGCTTGCCAGGCCTGCTGTCCTGGATGATTTTGTTAGTACGAT |
| | | TGATTTACCTAATTATGGATGCACAATTCCTGAAAAGACCAGTTG |
| | | CAGTGTTTATGGCTGGGGCTACACTGGATTGATCAACTATGATGG |
| | | CCTATTACGAGTGGCACATCTCTATATAATGGGAAATGAGAAATG |
| | | CAGCCAGCATCATCGAGGGAAGGTGACTCTGAATGAGTCTGAAA |
| | | TATGTGCTGGGGCTGAAAAGATTGGATCAGGACCATGTGAGGGG |
| | | GATTATGGTGGCCCACTTGTTTGTGAGCAACATAAAATGAGAATG |
| | | GTTCTTGGTGTCATTGTTCCTGGTCGTGGATGTGCCATTCCAAATC |
| | | GTCCTGGTATTTTTGTCCGAGTAGCATATTATGCAAAATGGATAC |
| | | ACAAAATTATTTTAACATATAAGGTACCACAGTCATAG |
| SEQ ID | Nucleotide | ATGTGGGTGACCAAACTCCTGCCAGCCCTGCTGCTGCAGCATGTC |
| NO. 27 | sequence of | CTCCTGCATCTCCTCCTGCTCCCCATCGCCATCCCCTATGCAGAGG |
| | HGF-X2 | GACAAAGGAAAAGAAGAAATACAATTCATGAATTCAAAAAATCA |
| | | GCAAAGACTACCCTAATCAAAATAGATCCAGCACTGAAGATAAA |
| | | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGGTAAGAACAG |
| | | TATGAAGAAAAGAGATGAAGCCTCTGTCTTTTTTACATGTTAACA |
| | | GTCTCATATTAGTCCTTCAGAATAATTCTACAATCCTAAAATAACT |
| | | TAGCCAACTTGCTGAATTGTATTACGGCAAGGTTTATATGAATTC |
| | | ATGACTGATATTTAGCAAATGATTAATTAATATGTTAATAAAATG |
| | | TAGCCAAAACAATATCTTACCTTAATGCCTCAATTTGTAGATCTC |
| | | GGTATTTGTGAAATAATAACGTAAACTTCGTTTAAAAGGATTCTT |
| | | CTTCCTGTCTTTGAGAAAGTACGGCACTGTGCAGGGGGAGAGGTT |
| | | GATTGTGAAAAATCAGAGGTAGATGAGAATCTTACTGAGGGCTG |
| | | AGGGTTCTTTAACCTTGGTGGATCTCAACATTGGTTGCACATTAA |
| | | AATCACCTGCTGCAAGCCCTTGACGAATCTTACTTAGAAGATGAC |
| | | AACACAGAACAATTAAATCAGAATCTCTGGGGAGAATAGGGCAC |
| | | CAGTATTTTTTGAGCTCCCACCATGATTCCAAAGTGCAGCCAAAT |
| | | TTGAGAACCACTGCTAAAAGCTCAAGCTTCAGATTGACCAGCTTT |
| | | TCCATCTCACCTATCGCCTAAAGACCAAATTGGATAAATGTGTTC |
| | | ATTACGACAGATGGGTACTATTTAAAGATGAGTAAACACAATATA |
| | | CTTAGGCTCGTCAGACTGAGAGTTTTAATCATCACTGAGGAAAAA |
| | | CATAGATATCTAATACTGACTGGAGTATTAGTCAAGGCTTATTTC |
| | | ACACACAATTTTATCAGAAACCAAAGTAGTTTAAAACAGCTCTCC |
| | | CCTTATTAGTAATGCATTGGAGGGTTTACTTTACCATGTACCTTGC |
| | | TGAGCACTGTACCTTGTTAATCTCATTTACTTGTAATGAGAACCAC |
| | | ACAGCGGGTAGTTTTATTGGTTCTATTTTACCTACATGACAAAACT |
| | | GAAGCATAAAAACACTTAGTAAGTTTTCAGTGTCATGCACAACTA |
| | | GGAAGTGACATGGCCAGAATATAAGCCCAGTCACCATCACTCTAT |
| | | AACCTGCGCTTTTAACAACTTCAGGGCATGACACATTTGGCCGGT |
| | | CAGTAGAACCCATGCTGTGATTTGTTTTTGCAGTGGTGGTGATGA |
| | | CTGCCTTGTTGAATCCACTTTTTATTCTATTCCATTTTGGGGACAC |
| | | AATTCTGCAAGATGATTCTTCATTAGGAAACAGAGATGAGTTATT |
| | | GACCAACACAGAAAGAAAAAGAGTTTGTTGCTCCACACTGGGAT |
| | | TAAACCTATGATCTTGGCCTAATTAACACTAGCTAGTAAGTGTCC |
| | | AAGCTGATCATCTCTACAACATTTCAATAACAGAAAACAACAATT |
| | | TTCAAAATTAGTTACTTACAATTATGTAGAAATGCCTCTAAAACA |
| | | CAGTATTTTCCTTATATTACAAAAACAAAAATTATAATTGGTTTTG |
| | | TCCTCTTTTGAGAGTTTGCATGGTGTTACTCCCTGCATAGTGAAGA |
| | | AAACATTTTATTTAAGTAGATGGATCTAAGTTTTTCATGAACAAA |
| | | GGAATGACATTTGAAATCAATCCTACCCTAGTCCAGGAGAATGCA |
| | | TTAGATTAACCTAGTAGAGGTCTTATTTCACCCTGAGTTTTCTATG |
| | | ATCGTGATTCTCTGCTGGAGGAGTAATTGTGAAATAGATCTCTCT |
| | | GGGAACTGGCTTCCTAGTCCAATCAGCTCTTTTACCAATGAACAC |
| | | TTCCTTGTGATATAGATGTTTATGGCCGAGAGGATCTCTTCCTTTC |
| | | TACCTGTATTTGTCCTAATAAATTGTTGACTTATTAATTCACTACT |
| | | TCCTCACAGCTTTTTTTTGGCTTTACAAATCCACTGGAAAGGTATA |
| | | TGGGTGTATCACTTTGTGTATTTCGGTGTGCATGTGTAGAGGGGA |
| | | CAAAAATCCTCTCTCAAACTATAAATATTGAGTATTTGTGTATTG |
| | | AACATTTGCTATAACTACTAGGTTTCTTAAATAATCTTAATATATA |
| | | AAATGATATAGAAAAAGGGAAATTATAGTTCGTATTATTCATCTA |
| | | AGTGAAGAGATTAAAACCCAGGGAGTAAATAAATTGTCTAAGGA |
| | | CTAAGGTTGTATACTATTTAGGTGATAGATATGGGGCAACCGTAT |
| | | GGGTTTTATGATTAACAAATAAACTTCTCACCACTCTACCATATC |
| | | AACTTTTCCATAAAAGAGAGCTATAGTATTCTTTGCTTAAATAAA |
| | | TTTGATTAGTGCATGACTTCTTGAAAACATATAAAGCAAAAGTCA |
| | | CATTTGATTCTATCAGAAAAGTGAGTAAGCCATGGCCCAAACAAA |
| | | AGATGCATTAAAATATTCTGGAATGATGGAGCTAAAAGTAAGAA |
| | | AAATGACTTTTTAAAAAAGTTTACTGTTAGGAATTGTGAAATTAT |
| | | GCTGAATTTTAGTTGCATTATAATTTTTGTCAGTCATACGGTCTGA |
| | | CAACCTGTCTTATTTCTATTTCCCCATATGAGGAATGCTAGTTAAG |
| | | TATGGATATTAACTATTACTACTTAGATGCATTGAAGTTGCATAA |
| | | TATGGATAATACTTCACTGGTTCCCTGAAAATGTTTAGTTAGTAAT |
| | | AAGTCTCTTACACTATTTGTTTTGTCCAATAATTTATATTTTCTGA |
| | | AGACTTAACTCTAGAATACACTCATGTCAAAATGAAAGAATTTCA |
| | | TTGCAAAATATTGCTTGGTACATGACGCATACCTGTATTTGTTTTG |
| | | TGTCACAACATGAAAAATGATGGTTTATTAGAAGTTTCATTGGGT |
| | | AGGAAACACATTTGAATGGTATTTACTAAGATACTAAAATCCTTG |
| | | GACTTCACTCTAATTTTAGTGCCATTTAGAACTCAAGGTCTCAGTA |
| | | AAAGTAGAAATAAAGCCTGTTAACAAAACACAAACTGAATATTA |
| | | AAAATGTAACTGGATTTTCAAAGAAATGTTTACTGGTATTACCTG |
| | | TAGATGTATATTCTTTATTATGATCTTTTGTGTAAAGTCTGGCAGA |
| | | CAAATGCAATATCTAATTGTTGAGTCCAATATCACAAGCAGTACA |
| | | AAAGTATAAAAAAGACTTGGCCTTTTCTAATGTGTTAAAATACTT |
| | | TATGCTGGTAATAACACTAAGAGTAGGGCACTAGAAATTTTAAGT |
| | | GAAGATAATGTGTTGCAGTTACTGCACTCAATGGCTTACTATTAT |
| | | AAACCAAAACTGGGATCACTAAGCTCCAGTCAGTCAAAATGATC |
| | | AAAATTATTGAAGAGAATAAGCAATTCTGTTCTTTATTAGGACAC |
| | | AGTAGATACAGACTACAAAGTGGAGTGTGCTTAATAAGAGGTAG |
| | | CATTTGTTAAGTGTCAATTACTCTATTATCCCTTGGAGCTTCTCAA |
| | | AATAACCATATAAGGTGTAAGATGTTAAAGGTTATGGTTACACTC |
| | | AGTGCACAGGTAAGCTAATAGGCTGAGAGAAGCTAAATTACTTA |
| | | CTGGGGTCTCACAGTAAGAAAGTGAGCTGAAGTTTCAGCCCAGAT |
| | | TTAACTGGATTCTGGGCTCTTTATTCATGTTACTTCATGAATCTGT |
| | | TTCTCAATTGTGCAGAAAAAAGGGGGCTATTTATAAGAAAAGCA |
| | | ATAAACAAACAAGTAATGATCTCAAATAAGTAATGCAAGAAATA |
| | | GTGAGATTTCAAAATCAGTGGCAGCGATTTCTCAGTTCTGTCCTA |
| | | AGTGGCCTTGCTCAATCACCTGCTATCTTTTAGTGGAGCTTTGAAA |
| | | TTATGTTTCAGACAACTTCGATTCAGTTCTAGAATGTTTGACTCAG |
| | | CAAATTCACAGGCTCATCTTTCTAACTTGATGGTGAATATGGAAA |
| | | TTCAGCTAAATGGATGTTAATAAAATTCAAACGTTTTAAGGACAG |
| | | ATGGAAATGACAGAATTTTAAGGTAAAATATATGAAGGAATATA |
| | | AGATAAAGGATTTTTCTACCTTCAGCAAAAACATACCCACTAATT |
| | | AGTAAAATTAATAGGCGAAAAAAAGTTGCATGCTCTTATACTGTA |
| | | ATGATTATCATTTTAAAACTAGCTTTTTGCCTTCGAGCTATCGGGG |
| | | TAAAGACCTACAGGAAAACTACTGTCGAAATCCTCGAGGGGAAG |
| | | AAGGGGGACCCTGGTGTTTCACAAGCAATCCAGAGGTACGCTAC |
| | | GAAGTCTGTGACATTCCTCAGTGTTCAGAAGTTGAATGCATGACC |
| | | TGCAATGGGGAGAGTTATCGAGGTCTCATGGATCATACAGAATCA |
| | | GGCAAGATTTGTCAGCGCTGGGATCATCAGACACCACACCGGCA |
| | | CAAATTCTTGCCTGAAAGATATCCCGACAAGGGCTTTGATGATAA |
| | | TTATTGCCGCAATCCCGATGGCCAGCCGAGGCCATGGTGCTATAC |
| | | TCTTGACCCTCACACCCGCTGGGAGTACTGTGCAATTAAAACATG |
| | | CGCTGACAATACTATGAATGACACTGATGTTCCTTTGGAAACAAC |
| | | TGAATGCATCCAAGGTCAAGGAGAAGGCTACAGGGGCACTGTCA |
| | | ATACCATTTGGAATGGAATTCCATGTCAGCGTTGGGATTCTCAGT |
| | | ATCCTCACGAGCATGACATGACTCCTGAAAATTTCAAGTGCAAGG |
| | | ACCTACGAGAAAATTACTGCCGAAATCCAGATGGGTCTGAATCAC |
| | | CCTGGTGTTTTACCACTGATCCAAACATCCGAGTTGGCTACTGCTC |
| | | CCAAATTCCAAACTGTGATATGTCACATGGACAAGATTGTTATCG |
| | | TGGGAATGGCAAAAATTATATGGGCAACTTATCCCAAACAAGAT |
| | | CTGGACTAACATGTTCAATGTGGGACAAGAACATGGAAGACTTA |
| | | CATCGTCATATCTTCTGGGAACCAGATGCAAGTAAGCTGAATGAG |
| | | AATTACTGCCGAAATCCAGATGATGATGCTCATGGACCCTGGTGC |
| | | TACACGGGAAATCCACTCATTCCTTGGGATTATTGCCCTATTTCTC |
| | | GTTGTGAAGGTGATACCACACCTACAATAGTCAATTTAGACCATC |
| | | CCGTAATATCTTGTGCCAAAACGAAACAATTGCGAGTTGTAAATG |
| | | GGATTCCAACACGAACAAACATAGGATGGATGGTTAGTTTGAGA |
| | | TACAGAAATAAACATATCTGCGGAGGATCATTGATAAAGGAGAG |
| | | TTGGGTTCTTACTGCACGACAGTGTTTCCCTTCTCGAGACTTGAAA |
| | | GATTATGAAGCTTGGCTTGGAATTCATGATGTCCACGGAAGAGGA |
| | | GATGAGAAATGCAAACAGGTTCTCAATGTTTCCCAGCTGGTATAT |
| | | GGCCCTGAAGGATCAGATCTGGTTTTAATGAAGCTTGCCAGGCCT |
| | | GCTGTCCTGGATGATTTTGTTAGTACGATTGATTTACCTAATTATG |
| | | GATGCACAATTCCTGAAAAGACCAGTTGCAGTGTTTATGGCTGGG |
| | | GCTACACTGGATTGATCAACTATGATGGCCTATTACGAGTGGCAC |
| | | ATCTCTATATAATGGGAAATGAGAAATGCAGCCAGCATCATCGA |
| | | GGGAAGGTGACTCTGAATGAGTCTGAAATATGTGCTGGGGCTGA |
| | | AAAGATTGGATCAGGACCATGTGAGGGGGATTATGGTGGCCCAC |
| | | TTGTTTGTGAGCAACATAAAATGAGAATGGTTCTTGGTGTCATTG |
| | | TTCCTGGTCGTGGATGTGCCATTCCAAATCGTCCTGGTATTTTTGT |
| | | CCGAGTAGCATATTATGCAAAATGGATACACAAAATTATTTTAAC |
| | | ATATAAGGTACCACAGTCATAG |
| SEQ ID | Nucleotide | ATGTGGGTGACCAAACTCCTGCCAGCCCTGCTGCTGCAGCATGTC |
| NO. 28 | sequence of | CTCCTGCATCTCCTCCTGCTCCCCATCGCCATCCCCTATGCAGAGG |
| | HGF-X3 | GACAAAGGAAAAGAAGAAATACAATTCATGAATTCAAAAAATCA |
| | | GCAAAGACTACCCTAATCAAAATAGATCCAGCACTGAAGATAAA |
| | | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGGTAAGAACAG |
| | | TATGAAGAAAAGAGATGAAGCCTCTGTCTTTTTTACATGTTAACA |
| | | GTCTCATATTAGTCCTTCAGAATAATTCTACAATCCTAAAATAACT |
| | | TAGCCAACTTGCTGAATTGTATTACGGCAAGGTTTATATGAATTC |
| | | ATGACTGATATTTAGCAAATGATTAATTAATATGTTAATAAAATG |
| | | TAGCCAAAACAATATCTTACCTTAATGCCTCAATTTGTAGATCTC |
| | | GGTATTTGTGAAATAATAACGTAAACTTCGTTTAAAAGGATTCTT |
| | | CTTCCTGTCTTTGAGAAAGTACGGCACTGTGCAGGGGGAGAGGTT |
| | | GATTGTGAAAAATCAGAGGTAGATGAGAATCTTACTGAGGGCTG |
| | | AGGGTTCTTTAACCTTGGTGGATCTCAACATTGGTTGCACATTAA |
| | | AATCACCTGCTGCAAGCCCTTGACGAATCTTACTTAGAAGATGAC |
| | | AACACAGAACAATTAAATCAGAATCTCTGGGGAGAATAGGGCAC |
| | | CAGTATTTTTTGAGCTCCCACCATGATTCCAAAGTGCAGCCAAAT |
| | | TTGAGAACCACTGCTAAAAGCTCAAGCTTCAGATTGACCAGCTTT |
| | | TCCATCTCACCTATCGCCTAAAGACCAAATTGGATAAATGTGTTC |
| | | ATTACGACAGATGGGTACTATTTAAAGATGAGTAAACACAATATA |
| | | CTTAGGCTCGTCAGACTGAGAGTTTTAATCATCACTGAGGAAAAA |
| | | CATAGATATCTAATACTGACTGGAGTATTAGTCAAGGCTTATTTC |
| | | ACACACAATTTTATCAGAAACCAAAGTAGTTTAAAACAGCTCTCC |
| | | CCTTATTAGTAATGCATTGGAGGGTTTACTTTACCATGTACCTTGC |
| | | TGAGCACTGTACCTTGTTAATCTCATTTACTTGTAATGAGAACCAC |
| | | ACAGCGGGTAGTTTTATTGGTTCTATTTTACCTACATGACAAAACT |
| | | GAAGCATAAAAACACTTAGTAAGTTTTCAGTGTCATGCACAACTA |
| | | GGAAGTGACATGGCCAGAATATAAGCCCAGTCACCATCACTCTAT |
| | | AACCTGCGCTTTTAACAACTTCAGGGCATGACACATTTGGCCGGT |
| | | CAGTAGAACCCATGCTGTGATTTGTTTTTGCAGTGGTGGTGATGA |
| | | CTGCCTTGTTGAATCCACTTTTTATTCTATTCCATTTTGGGGACAC |
| | | AATTCTGCAAGATGATTCTTCATTAGGAAACAGAGATGAGTTATT |
| | | GACCAACACAGAAAGAAAAAGAGTTTGTTGCTCCACACTGGGAT |
| | | TAAACCTATGATCTTGGCCTAATTAACACTAGCTAGTAAGTGTCC |
| | | AAGCTGATCATCTCTACAACATTTCAATAACAGAAAACAACAATT |
| | | TTCAAAATTAGTTACTTACAATTATGTAGAAATGCCTCTAAAACA |
| | | CAGTATTTTCCTTATATTACAAAAACAAAAATTATAATTGGTTTTG |
| | | TCCTCTTTTGAGAGTTTGCATGGTGTTACTCCCTGCATAGTGAAGA |
| | | AAACATTTTATTTAAGTAGATGGATCTAAGTTTTTCATGAACAAA |
| | | GGAATGACATTTGAAATCAATCCTACCCTAGTCCAGGAGAATGCA |
| | | TTAGATTAACCTAGTAGAGGTCTTATTTCACCCTGAGTTTTCTATG |
| | | ATCGTGATTCTCTGCTGGAGGAGTAATTGTGAAATAGATCTCTCT |
| | | GGGAACTGGCTTCCTAGTCCAATCAGCTCTTTTACCAATGAACAC |
| | | TTCCTTGTGATATAGATGTTTATGGCCGAGAGGATCCTGGGTAGG |
| | | AAACACATTTGAATGGTATTTACTAAGATACTAAAATCCTTGGAC |
| | | TTCACTCTAATTTTAGTGCCATTTAGAACTCAAGGTCTCAGTAAA |
| | | AGTAGAAATAAAGCCTGTTAACAAAACACAAACTGAATATTAAA |
| | | AATGTAACTGGATTTTCAAAGAAATGTTTACTGGTATTACCTGTA |
| | | GATGTATATTCTTTATTATGATCTTTTGTGTAAAGTCTGGCAGACA |
| | | AATGCAATATCTAATTGTTGAGTCCAATATCACAAGCAGTACAAA |
| | | AGTATAAAAAAGACTTGGCCTTTTCTAATGTGTTAAAATACTTTA |
| | | TGCTGGTAATAACACTAAGAGTAGGGCACTAGAAATTTTAAGTGA |
| | | AGATAATGTGTTGCAGTTACTGCACTCAATGGCTTACTATTATAA |
| | | ACCAAAACTGGGATCACTAAGCTCCAGTCAGTCAAAATGATCAA |
| | | AATTATTGAAGAGAATAAGCAATTCTGTTCTTTATTAGGACACAG |
| | | TAGATACAGACTACAAAGTGGAGTGTGCTTAATAAGAGGTAGCA |
| | | TTTGTTAAGTGTCAATTACTCTATTATCCCTTGGAGCTTCTCAAAA |
| | | TAACCATATAAGGTGTAAGATGTTAAAGGTTATGGTTACACTCAG |
| | | TGCACAGGTAAGCTAATAGGCTGAGAGAAGCTAAATTACTTACTG |
| | | GGGTCTCACAGTAAGAAAGTGAGCTGAAGTTTCAGCCCAGATTTA |
| | | ACTGGATTCTGGGCTCTTTATTCATGTTACTTCATGAATCTGTTTC |
| | | TCAATTGTGCAGAAAAAAGGGGGCTATTTATAAGAAAAGCAATA |
| | | AACAAACAAGTAATGATCTCAAATAAGTAATGCAAGAAATAGTG |
| | | AGATTTCAAAATCAGTGGCAGCGATTTCTCAGTTCTGTCCTAAGT |
| | | GGCCTTGCTCAATCACCTGCTATCTTTTAGTGGAGCTTTGAAATTA |
| | | TGTTTCAGACAACTTCGATTCAGTTCTAGAATGTTTGACTCAGCA |
| | | AATTCACAGGCTCATCTTTCTAACTTGATGGTGAATATGGAAATT |
| | | CAGCTAAATGGATGTTAATAAAATTCAAACGTTTTAAGGACAGAT |
| | | GGAAATGACAGAATTTTAAGGTAAAATATATGAAGGAATATAAG |
| | | ATAAAGGATTTTTCTACCTTCAGCAAAAACATACCCACTAATTAG |
| | | TAAAATTAATAGGCGAAAAAAAGTTGCATGCTCTTATACTGTAAT |
| | | GATTATCATTTTAAAACTAGCTTTTTGCCTTCGAGCTATCGGGGTA |
| | | AAGACCTACAGGAAAACTACTGTCGAAATCCTCGAGGGGAAGAA |
| | | GGGGGACCCTGGTGTTTCACAAGCAATCCAGAGGTACGCTACGA |
| | | AGTCTGTGACATTCCTCAGTGTTCAGAAGTTGAATGCATGACCTG |
| | | CAATGGGGAGAGTTATCGAGGTCTCATGGATCATACAGAATCAG |
| | | GCAAGATTTGTCAGCGCTGGGATCATCAGACACCACACCGGCAC |
| | | AAATTCTTGCCTGAAAGATATCCCGACAAGGGCTTTGATGATAAT |
| | | TATTGCCGCAATCCCGATGGCCAGCCGAGGCCATGGTGCTATACT |
| | | CTTGACCCTCACACCCGCTGGGAGTACTGTGCAATTAAAACATGC |
| | | GCTGACAATACTATGAATGACACTGATGTTCCTTTGGAAACAACT |
| | | GAATGCATCCAAGGTCAAGGAGAAGGCTACAGGGGCACTGTCAA |
| | | TACCATTTGGAATGGAATTCCATGTCAGCGTTGGGATTCTCAGTA |
| | | TCCTCACGAGCATGACATGACTCCTGAAAATTTCAAGTGCAAGGA |
| | | CCTACGAGAAAATTACTGCCGAAATCCAGATGGGTCTGAATCACC |
| | | CTGGTGTTTTACCACTGATCCAAACATCCGAGTTGGCTACTGCTCC |
| | | CAAATTCCAAACTGTGATATGTCACATGGACAAGATTGTTATCGT |
| | | GGGAATGGCAAAAATTATATGGGCAACTTATCCCAAACAAGATC |
| | | TGGACTAACATGTTCAATGTGGGACAAGAACATGGAAGACTTAC |
| | | ATCGTCATATCTTCTGGGAACCAGATGCAAGTAAGCTGAATGAGA |
| | | ATTACTGCCGAAATCCAGATGATGATGCTCATGGACCCTGGTGCT |
| | | ACACGGGAAATCCACTCATTCCTTGGGATTATTGCCCTATTTCTCG |
| | | TTGTGAAGGTGATACCACACCTACAATAGTCAATTTAGACCATCC |
| | | CGTAATATCTTGTGCCAAAACGAAACAATTGCGAGTTGTAAATGG |
| | | GATTCCAACACGAACAAACATAGGATGGATGGTTAGTTTGAGAT |
| | | ACAGAAATAAACATATCTGCGGAGGATCATTGATAAAGGAGAGT |
| | | TGGGTTCTTACTGCACGACAGTGTTTCCCTTCTCGAGACTTGAAA |
| | | GATTATGAAGCTTGGCTTGGAATTCATGATGTCCACGGAAGAGGA |
| | | GATGAGAAATGCAAACAGGTTCTCAATGTTTCCCAGCTGGTATAT |
| | | GGCCCTGAAGGATCAGATCTGGTTTTAATGAAGCTTGCCAGGCCT |
| | | GCTGTCCTGGATGATTTTGTTAGTACGATTGATTTACCTAATTATG |
| | | GATGCACAATTCCTGAAAAGACCAGTTGCAGTGTTTATGGCTGGG |
| | | GCTACACTGGATTGATCAACTATGATGGCCTATTACGAGTGGCAC |
| | | ATCTCTATATAATGGGAAATGAGAAATGCAGCCAGCATCATCGA |
| | | GGGAAGGTGACTCTGAATGAGTCTGAAATATGTGCTGGGGCTGA |
| | | AAAGATTGGATCAGGACCATGTGAGGGGGATTATGGTGGCCCAC |
| | | TTGTTTGTGAGCAACATAAAATGAGAATGGTTCTTGGTGTCATTG |
| | | TTCCTGGTCGTGGATGTGCCATTCCAAATCGTCCTGGTATTTTTGT |
| | | CCGAGTAGCATATTATGCAAAATGGATACACAAAATTATTTTAAC |
| | | ATATAAGGTACCACAGTCATAG |
| SEQ ID | Nucleotide | ATGTGGGTGACCAAACTCCTGCCAGCCCTGCTGCTGCAGCATGTC |
| NO. 29 | sequence of | CTCCTGCATCTCCTCCTGCTCCCCATCGCCATCCCCTATGCAGAGG |
| | HGF-X4 | GACAAAGGAAAAGAAGAAATACAATTCATGAATTCAAAAAATCA |
| | | GCAAAGACTACCCTAATCAAAATAGATCCAGCACTGAAGATAAA |
| | | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGGTAAGAACAG |
| | | TATGAAGAAAAGAGATGAAGCCTCTGTCTTTTTTACATGTTAACA |
| | | GTCTCATATTAGTCCTTCAGAATAATTCTACAATCCTAAAATAACT |
| | | TAGCCAACTTGCTGAATTGTATTACGGCAAGGTTTATATGAATTC |
| | | ATGACTGATATTTAGCAAATGATTAATTAATATGTTAATAAAATG |
| | | TAGCCAAAACAATATCTTACCTTAATGCCTCAATTTGTAGATCTC |
| | | GGTATTTGTGAAATAATAACGTAAACTTCGTTTAAAAGGATTCTT |
| | | CTTCCTGTCTTTGAGAAAGTACGGCACTGTGCAGGGGGAGAGGTT |
| | | GATTGTGAAAAATCAGAGGTAGATGAGAATCTTACTGAGGGCTG |
| | | AGGGTTCTTTAACCTTGGTGGATCTCAACATTGGTTGCACATTAA |
| | | AATCACCTGCTGCAAGCCCTTGACGAATCTTACTTAGAAGATGAC |
| | | AACACAGAACAATTAAATCAGAATCTCTGGGGAGAATAGGGCAC |
| | | CAGTATTTTTTGAGCTCCCACCATGATTCCAAAGTGCAGCCAAAT |
| | | TTGAGAACCACTGCTAAAAGCTCAAGCTTCAGATTGACCAGCTTT |
| | | TCCATCTCACCTATCGCCTAAAGACCAAATTGGATAAATGTGTTC |
| | | ATTACGACAGATGGGTACTATTTAAAGATGAGTAAACACAATATA |
| | | CTTAGGCTCGTCAGACTGAGAGTTTTAATCATCACTGAGGAAAAA |
| | | CATAGATATCTAATACTGACTGGAGTATTAGTCAAGGCTTATTTC |
| | | ACACACAATTTTATCAGAAACCAAAGTAGTTTAAAACAGCTCTCC |
| | | CCTTATTAGTAATGCATTGGAGGGTTTACTTTACCATGTACCTTGC |
| | | TGAGCACTGTACCTTGTTAATCTCATTTACTTGTAATGAGAACCAC |
| | | ACAGCGGGTAGTTTTATTGGTTCTATTTTACCTACATGACAAAACT |
| | | GAAGCATAAAAACACTTAGTAAGTTTTCAGTGTCATGCACAACTA |
| | | GGAAGTGACATGGCCAGAATATAAGCCCAGTCACCATCACTCTAT |
| | | AACCTGCGCTTTTAACAACTTCAGGGCATGACACATTTGGCCGGT |
| | | CAGTAGAACCCATGCTGTGATTTGTTTTTGCAGTGGTGGTGATGA |
| | | CTGCCTTGTTGAATCCACTTTTTATTCTATTCCATTTTGGGGACAC |
| | | AATTCTGCAAGATGATTCTTCATTAGGAAACAGAGATGAGTTATT |
| | | GACCAACACAGAAAGAAAAAGAGTTTGTTGCTCCACACTGGGAT |
| | | TAAACCTATGATCTTGGCCTAATTAACACTAGCTAGTAAGTGTCC |
| | | AAGCTGATCATCTCTACAACATTTCAATAACAGAAAACAACAATT |
| | | TTCAAAATTAGTTACTTACAATTATGTAGAAATGCCTCTAAAACA |
| | | CAGTATTTTCCTTATATTACAAAAACAAAAATTATAATTGGTTTTG |
| | | TCCTCTTTTGAGAGTTTGCATGGTGTTACTCCCTGCATAGTGAAGA |
| | | AAACATTTTATTTAAGTAGATGGATCTAAGTTTTTCATGAACAAA |
| | | GGAATGACATTTGAAATCAATCCTACCCTAGTCCAGGAGAATGCA |
| | | TTAGATTAACCTAGTAGAGGTCTTATTTCACCCTGAGTTTTCTATG |
| | | ATCGTGATTCTCTGCTGGAGGAGTAATTGTGAAATAGATCTCTCT |
| | | GGGAACTGGCTTCCTAGTCCAATCAGCTCTTTTACCAATGAACAC |
| | | TTCCTTGTGATATAGATGTTTATGGCCGAGAGGATCCTTATGTTTC |
| | | AGACAACTTCGATTCAGTTCTAGAATGTTTGACTCAGCAAATTCA |
| | | CAGGCTCATCTTTCTAACTTGATGGTGAATATGGAAATTCAGCTA |
| | | AATGGATGTTAATAAAATTCAAACGTTTTAAGGACAGATGGAAAT |
| | | GACAGAATTTTAAGGTAAAATATATGAAGGAATATAAGATAAAG |
| | | GATTTTTCTACCTTCAGCAAAAACATACCCACTAATTAGTAAAAT |
| | | TAATAGGCGAAAAAAAGTTGCATGCTCTTATACTGTAATGATTAT |
| | | CATTTTAAAACTAGCTTTTTGCCTTCGAGCTATCGGGGTAAAGAC |
| | | CTACAGGAAAACTACTGTCGAAATCCTCGAGGGGAAGAAGGGGG |
| | | ACCCTGGTGTTTCACAAGCAATCCAGAGGTACGCTACGAAGTCTG |
| | | TGACATTCCTCAGTGTTCAGAAGTTGAATGCATGACCTGCAATGG |
| | | GGAGAGTTATCGAGGTCTCATGGATCATACAGAATCAGGCAAGA |
| | | TTTGTCAGCGCTGGGATCATCAGACACCACACCGGCACAAATTCT |
| | | TGCCTGAAAGATATCCCGACAAGGGCTTTGATGATAATTATTGCC |
| | | GCAATCCCGATGGCCAGCCGAGGCCATGGTGCTATACTCTTGACC |
| | | CTCACACCCGCTGGGAGTACTGTGCAATTAAAACATGCGCTGACA |
| | | ATACTATGAATGACACTGATGTTCCTTTGGAAACAACTGAATGCA |
| | | TCCAAGGTCAAGGAGAAGGCTACAGGGGCACTGTCAATACCATT |
| | | TGGAATGGAATTCCATGTCAGCGTTGGGATTCTCAGTATCCTCAC |
| | | GAGCATGACATGACTCCTGAAAATTTCAAGTGCAAGGACCTACG |
| | | AGAAAATTACTGCCGAAATCCAGATGGGTCTGAATCACCCTGGTG |
| | | TTTTACCACTGATCCAAACATCCGAGTTGGCTACTGCTCCCAAATT |
| | | CCAAACTGTGATATGTCACATGGACAAGATTGTTATCGTGGGAAT |
| | | GGCAAAAATTATATGGGCAACTTATCCCAAACAAGATCTGGACTA |
| | | ACATGTTCAATGTGGGACAAGAACATGGAAGACTTACATCGTCAT |
| | | ATCTTCTGGGAACCAGATGCAAGTAAGCTGAATGAGAATTACTGC |
| | | CGAAATCCAGATGATGATGCTCATGGACCCTGGTGCTACACGGGA |
| | | AATCCACTCATTCCTTGGGATTATTGCCCTATTTCTCGTTGTGAAG |
| | | GTGATACCACACCTACAATAGTCAATTTAGACCATCCCGTAATAT |
| | | CTTGTGCCAAAACGAAACAATTGCGAGTTGTAAATGGGATTCCAA |
| | | CACGAACAAACATAGGATGGATGGTTAGTTTGAGATACAGAAAT |
| | | AAACATATCTGCGGAGGATCATTGATAAAGGAGAGTTGGGTTCTT |
| | | ACTGCACGACAGTGTTTCCCTTCTCGAGACTTGAAAGATTATGAA |
| | | GCTTGGCTTGGAATTCATGATGTCCACGGAAGAGGAGATGAGAA |
| | | ATGCAAACAGGTTCTCAATGTTTCCCAGCTGGTATATGGCCCTGA |
| | | AGGATCAGATCTGGTTTTAATGAAGCTTGCCAGGCCTGCTGTCCT |
| | | GGATGATTTTGTTAGTACGATTGATTTACCTAATTATGGATGCAC |
| | | AATTCCTGAAAAGACCAGTTGCAGTGTTTATGGCTGGGGCTACAC |
| | | TGGATTGATCAACTATGATGGCCTATTACGAGTGGCACATCTCTA |
| | | TATAATGGGAAATGAGAAATGCAGCCAGCATCATCGAGGGAAGG |
| | | TGACTCTGAATGAGTCTGAAATATGTGCTGGGGCTGAAAAGATTG |
| | | GATCAGGACCATGTGAGGGGGATTATGGTGGCCCACTTGTTTGTG |
| | | AGCAACATAAAATGAGAATGGTTCTTGGTGTCATTGTTCCTGGTC |
| | | GTGGATGTGCCATTCCAAATCGTCCTGGTATTTTTGTCCGAGTAGC |
| | | ATATTATGCAAAATGGATACACAAAATTATTTTAACATATAAGGT |
| | | ACCACAGTCATAG |
| SEQ ID | Nucleotide | ATGTGGGTGACCAAACTCCTGCCAGCCCTGCTGCTGCAGCATGTC |
| NO. 30 | sequence of | CTCCTGCATCTCCTCCTGCTCCCCATCGCCATCCCCTATGCAGAGG |
| | HGF-X5 | GACAAAGGAAAAGAAGAAATACAATTCATGAATTCAAAAAATCA |
| | | GCAAAGACTACCCTAATCAAAATAGATCCAGCACTGAAGATAAA |
| | | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGGTAAGAACAG |
| | | TATGAAGAAAAGAGATGAAGCCTCTGTCTTTTTTACATGTTAACA |
| | | GTCTCATATTAGTCCTTCAGAATAATTCTACAATCCTAAAATAACT |
| | | TAGCCAACTTGCTGAATTGTATTACGGCAAGGTTTATATGAATTC |
| | | ATGACTGATATTTAGCAAATGATTAATTAATATGTTAATAAAATG |
| | | TAGCCAAAACAATATCTTACCTTAATGCCTCAATTTGTAGATCTC |
| | | GGTATTTGTGGATCCAGTATATTAATAAAATCCCTTTTTGTATTCA |
| | | ATGAGGGAAACACATAATTTTCATCAATTAGCAGCTTATTGGAAT |
| | | ATCTGCATGATGGTTTAACACTTTTAAGTGTTGACTAAAGATTAA |
| | | TTTTACAGAAAATAGAAAAAGAAATATGTTTCTGTCTGGAGGAAT |
| | | GATTTATTGTTGACCCCTAAATTGAAATATTTTACTAGTGGCTTAA |
| | | TGGAAAGATGATGAAAGATGATGAAATTAATGTAGAAGCTTAAC |
| | | TAGAAAATCAGGTGACCTGATATCTACATCTGTATCCTTCATTGG |
| | | CCACCCAGCATTCATTAATGAATCAGATGATGGAATAGATCAAGT |
| | | TTCCTAGGAACACAGTGAATATTAAAAGAAAACAAAGGGAGCCT |
| | | AGCACCTAGAAGACCTAGTTTATATTTCAAAGTATATTTGGATGT |
| | | AACCCAATTTTAAACATTTCCTCACTTGTCTCTCTTAAAGCCTTGC |
| | | CAACAGCAAGGACAGAGAACCAAAAATAGTGTATATATGAATAA |
| | | ATGCTTATTACAGAATCTGCTGACTGGCACATGCTTTGTGTGTAAT |
| | | GGGTTCTCATAAACACTTGTTGAATGAACACACATAAGTGAAAGA |
| | | GCATGGCTAGGCTTCATCCCTTGGTCAAATATGGGGTGCTAAAGA |
| | | AAAGCAGGGGAAATACATTGGGACACTAACAAAAAAAAACAGTT |
| | | AATTTAGGTAAAAGATAAAATACACCACAGAATGAAGAAAAGAG |
| | | ATGACCCAGACTGCTCTTTAACCTTCATGTCCTAGAGAGGTTTTTG |
| | | ATATGAATTGCATTCAGAATTGTGGAAAGGAGCCCATCTTTTCTC |
| | | TTCATTTTGATTTTATTAACTCCAATGGGGGAATTTTATTCGTGTT |
| | | TTGGCCATATCTACTTTTGATTTCTACATTATTCTCTCTTCCTTTCT |
| | | ACCTGTATTTGTCCTAATAAATTGTTGACTTATTAATTCACTACTT |
| | | CCTCACAGCTTTTTTTTGGCTTTACAAATCCACTGGAAAGGTATAT |
| | | GGGTGTATCACTTTGTGTATTTCGGTGTGCATGTGTAGAGGGGAC |
| | | AAAAATCCTCTCTCAAACTATAAATATTGAGTATTTGTGTATTGA |
| | | ACATTTGCTATAACTACTAGGTTTCTTAAATAATCTTAATATATAA |
| | | AATGATATAGAAAAAGGGAAATTATAGTTCGTATTATTCATCTAA |
| | | GTGAAGAGATTAAAACCCAGGGAGTAAATAAATTGTCTAAGGAC |
| | | TAAGGTTGTATACTATTTAGGTGATAGATATGGGGCAACCGTATG |
| | | GGTTTTATGATTAACAAATAAACTTCTCACCACTCTACCATATCA |
| | | ACTTTTCCATAAAAGAGAGCTATAGTATTCTTTGCTTAAATAAATT |
| | | TGATTAGTGCATGACTTCTTGAAAACATATAAAGCAAAAGTCACA |
| | | TTTGATTCTATCAGAAAAGTGAGTAAGCCATGGCCCAAACAAAA |
| | | GATGCATTAAAATATTCTGGAATGATGGAGCTAAAAGTAAGAAA |
| | | AATGACTTTTTAAAAAAGTTTACTGTTAGGAATTGTGAAATTATG |
| | | CTGAATTTTAGTTGCATTATAATTTTTGTCAGTCATACGGTCTGAC |
| | | AACCTGTCTTATTTCTATTTCCCCATATGAGGAATGCTAGTTAAGT |
| | | ATGGATATTAACTATTACTACTTAGATGCATTGAAGTTGCATAAT |
| | | ATGGATAATACTTCACTGGTTCCCTGAAAATGTTTAGTTAGTAAT |
| | | AAGTCTCTTACACTATTTGTTTTGTCCAATAATTTATATTTTCTGA |
| | | AGACTTAACTCTAGAATACACTCATGTCAAAATGAAAGAATTTCA |
| | | TTGCAAAATATTGCTTGGTACATGACGCATACCTGTATTTGTTTTG |
| | | TGTCACAACATGAAAAATGATGGTTTATTAGAAGTTTCATTGGGT |
| | | AGGAAACACATTTGAATGGTATTTACTAAGATACTAAAATCCTTG |
| | | GACTTCACTCTAATTTTAGTGCCATTTAGAACTCAAGGTCTCAGTA |
| | | AAAGTAGAAATAAAGCCTGTTAACAAAACACAAACTGAATATTA |
| | | AAAATGTAACTGGATTTTCAAAGAAATGTTTACTGGTATTACCTG |
| | | TAGATGTATATTCTTTATTATGATCTTTTGTGTAAAGTCTGGCAGA |
| | | CAAATGCAATATCTAATTGTTGAGTCCAATATCACAAGCAGTACA |
| | | AAAGTATAAAAAAGACTTGGCCTTTTCTAATGTGTTAAAATACTT |
| | | TATGCTGGTAATAACACTAAGAGTAGGGCACTAGAAATTTTAAGT |
| | | GAAGATAATGTGTTGCAGTTACTGCACTCAATGGCTTACTATTAT |
| | | AAACCAAAACTGGGATCACTAAGCTCCAGTCAGTCAAAATGATC |
| | | AAAATTATTGAAGAGAATAAGCAATTCTGTTCTTTATTAGGACAC |
| | | AGTAGATACAGACTACAAAGTGGAGTGTGCTTAATAAGAGGTAG |
| | | CATTTGTTAAGTGTCAATTACTCTATTATCCCTTGGAGCTTCTCAA |
| | | AATAACCATATAAGGTGTAAGATGTTAAAGGTTATGGTTACACTC |
| | | AGTGCACAGGTAAGCTAATAGGCTGAGAGAAGCTAAATTACTTA |
| | | CTGGGGTCTCACAGTAAGAAAGTGAGCTGAAGTTTCAGCCCAGAT |
| | | TTAACTGGATTCTGGGCTCTTTATTCATGTTACTTCATGAATCTGT |
| | | TTCTCAATTGTGCAGAAAAAAGGGGGCTATTTATAAGAAAAGCA |
| | | ATAAACAAACAAGTAATGATCTCAAATAAGTAATGCAAGAAATA |
| | | GTGAGATTTCAAAATCAGTGGCAGCGATTTCTCAGTTCTGTCCTA |
| | | AGTGGCCTTGCTCAATCACCTGCTATCTTTTAGTGGAGCTTTGAAA |
| | | TTATGTTTCAGACAACTTCGATTCAGTTCTAGAATGTTTGACTCAG |
| | | CAAATTCACAGGCTCATCTTTCTAACTTGATGGTGAATATGGAAA |
| | | TTCAGCTAAATGGATGTTAATAAAATTCAAACGTTTTAAGGACAG |
| | | ATGGAAATGACAGAATTTTAAGGTAAAATATATGAAGGAATATA |
| | | AGATAAAGGATTTTTCTACCTTCAGCAAAAACATACCCACTAATT |
| | | AGTAAAATTAATAGGCGAAAAAAAGTTGCATGCTCTTATACTGTA |
| | | ATGATTATCATTTTAAAACTAGCTTTTTGCCTTCGAGCTATCGGGG |
| | | TAAAGACCTACAGGAAAACTACTGTCGAAATCCTCGAGGGGAAG |
| | | AAGGGGGACCCTGGTGTTTCACAAGCAATCCAGAGGTACGCTAC |
| | | GAAGTCTGTGACATTCCTCAGTGTTCAGAAGTTGAATGCATGACC |
| | | TGCAATGGGGAGAGTTATCGAGGTCTCATGGATCATACAGAATCA |
| | | GGCAAGATTTGTCAGCGCTGGGATCATCAGACACCACACCGGCA |
| | | CAAATTCTTGCCTGAAAGATATCCCGACAAGGGCTTTGATGATAA |
| | | TTATTGCCGCAATCCCGATGGCCAGCCGAGGCCATGGTGCTATAC |
| | | TCTTGACCCTCACACCCGCTGGGAGTACTGTGCAATTAAAACATG |
| | | CGCTGACAATACTATGAATGACACTGATGTTCCTTTGGAAACAAC |
| | | TGAATGCATCCAAGGTCAAGGAGAAGGCTACAGGGGCACTGTCA |
| | | ATACCATTTGGAATGGAATTCCATGTCAGCGTTGGGATTCTCAGT |
| | | ATCCTCACGAGCATGACATGACTCCTGAAAATTTCAAGTGCAAGG |
| | | ACCTACGAGAAAATTACTGCCGAAATCCAGATGGGTCTGAATCAC |
| | | CCTGGTGTTTTACCACTGATCCAAACATCCGAGTTGGCTACTGCTC |
| | | CCAAATTCCAAACTGTGATATGTCACATGGACAAGATTGTTATCG |
| | | TGGGAATGGCAAAAATTATATGGGCAACTTATCCCAAACAAGAT |
| | | CTGGACTAACATGTTCAATGTGGGACAAGAACATGGAAGACTTA |
| | | CATCGTCATATCTTCTGGGAACCAGATGCAAGTAAGCTGAATGAG |
| | | AATTACTGCCGAAATCCAGATGATGATGCTCATGGACCCTGGTGC |
| | | TACACGGGAAATCCACTCATTCCTTGGGATTATTGCCCTATTTCTC |
| | | GTTGTGAAGGTGATACCACACCTACAATAGTCAATTTAGACCATC |
| | | CCGTAATATCTTGTGCCAAAACGAAACAATTGCGAGTTGTAAATG |
| | | GGATTCCAACACGAACAAACATAGGATGGATGGTTAGTTTGAGA |
| | | TACAGAAATAAACATATCTGCGGAGGATCATTGATAAAGGAGAG |
| | | TTGGGTTCTTACTGCACGACAGTGTTTCCCTTCTCGAGACTTGAAA |
| | | GATTATGAAGCTTGGCTTGGAATTCATGATGTCCACGGAAGAGGA |
| | | GATGAGAAATGCAAACAGGTTCTCAATGTTTCCCAGCTGGTATAT |
| | | GGCCCTGAAGGATCAGATCTGGTTTTAATGAAGCTTGCCAGGCCT |
| | | GCTGTCCTGGATGATTTTGTTAGTACGATTGATTTACCTAATTATG |
| | | GATGCACAATTCCTGAAAAGACCAGTTGCAGTGTTTATGGCTGGG |
| | | GCTACACTGGATTGATCAACTATGATGGCCTATTACGAGTGGCAC |
| | | ATCTCTATATAATGGGAAATGAGAAATGCAGCCAGCATCATCGA |
| | | GGGAAGGTGACTCTGAATGAGTCTGAAATATGTGCTGGGGCTGA |
| | | AAAGATTGGATCAGGACCATGTGAGGGGGATTATGGTGGCCCAC |
| | | TTGTTTGTGAGCAACATAAAATGAGAATGGTTCTTGGTGTCATTG |
| | | TTCCTGGTCGTGGATGTGCCATTCCAAATCGTCCTGGTATTTTTGT |
| | | CCGAGTAGCATATTATGCAAAATGGATACACAAAATTATTTTAAC |
| | | ATATAAGGTACCACAGTCATAG |
| SEQ ID | Nucleotide | ATGTGGGTGACCAAACTCCTGCCAGCCCTGCTGCTGCAGCATGTC |
| NO. 31 | sequence of | CTCCTGCATCTCCTCCTGCTCCCCATCGCCATCCCCTATGCAGAGG |
| | HGF-X6 | GACAAAGGAAAAGAAGAAATACAATTCATGAATTCAAAAAATCA |
| | | GCAAAGACTACCCTAATCAAAATAGATCCAGCACTGAAGATAAA |
| | | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGGTAAGAACAG |
| | | TATGAAGAAAAGAGATGAAGCCTCTGTCTTTTTTACATGTTAACA |
| | | GTCTCATATTAGTCCTTCAGAATAATTCTACAATCCTAAAATAACT |
| | | TAGCCAACTTGCTGAATTGTATTACGGCAAGGTTTATATGAATTC |
| | | ATGACTGATATTTAGCAAATGATTAATTAATATGTTAATAAAATG |
| | | TAGCCAAAACAATATCTTACCTTAATGCCTCAATTTGTAGATCTC |
| | | GGTATTTGTGGATCTCTTCCTTTCTACCTGTATTTGTCCTAATAAA |
| | | TTGTTGACTTATTAAT(C ≥ T)TCACTACTTCCTCACAGCTTTTTTTT |
| | | GGCTTTACAAATCCACTGGAAAGGTATATGGGTGTATCACTTTGT |
| | | GTATTTCGGTGTGCATGTGTAGAGGGGACAAAAATCCTCTCTCAA |
| | | ACTATAAATATTGAGTATTTGTGTATTGAACATTTGCTATAACTAC |
| | | TAGGTTTCTTAAATAATCTTAATATATAAAATGATATAGAAAAAG |
| | | GGAAATTATAGTTCGTATTATTCATCTAAGTGAAGAGATTAAAAC |
| | | CCAGGGAGTAAATAAATTGTCTAAGGACTAAGGTTGTATACTATT |
| | | TAGGTGATAGATATGGGGCAACCGTATGGGTTTTATGATTAACAA |
| | | ATAAACTTCTCACCACTCTACCATATCAACTTTTCCATAAAAGAG |
| | | AGCTATAGTATTCTTTGCTTAAATAAATTTGATTAGTGCATGACTT |
| | | CTTGAAAACATATAAAGCAAAAGTCACATTTGATTCTATCAGAAA |
| | | AGTGAGTAAGCCATGGCCCAAACAAAAGATGCATTAAAATATTC |
| | | TGGAATGATGGAGCTAAAAGTAAGAAAAATGACTTTTTAAAAAA |
| | | GTTTACTGTTAGGAATTGTGAAATTATGCTGAATTTTAGTTGCATT |
| | | ATAATTTTTGTCAGTCATACGGTCTGACAACCTGTCTTATTTCTAT |
| | | TTCCCCATATGAGGAATGCTAGTTAAGTATGGATATTAACTATTA |
| | | CTACTTAGATGCATTGAAGTTGCATAATATGGATAATACTTCACT |
| | | GGTTCCCTGAAAATGTTTAGTTAGTAATAAGTCTCTTACACTATTT |
| | | GTTTTGTCCAATAATTTATATTTTCTGAAGACTTAACTCTAGAATA |
| | | CACTCATGTCAAAATGAAAGAATTTCATTGCAAAATATTGCTTGG |
| | | TACATGACGCATACCTGTATTTGTTTTGTGTCACAACATGAAAAA |
| | | TGATGGTTTATTAGAAGTTTCATTGGGTAGGAAACACATTTGAAT |
| | | GGTATTTACTAAGATACTAAAATCCTTGGACTTCACTCTAATTTTA |
| | | GTGCCATTTAGAACTCAAGGTCTCAGTAAAAGTAGAAATAAAGC |
| | | CTGTTAACAAAACACAAACTGAATATTAAAAATGTAACTGGATTT |
| | | TCAAAGAAATGTTTACTGGTATTACCTGTAGATGTATATTCTTTAT |
| | | TATGATCTTTTGTGTAAAGTCTGGCAGACAAATGCAATATCTAAT |
| | | TGTTGAGTCCAATATCACAAGCAGTACAAAAGTATAAAAAAGAC |
| | | TTGGCCTTTTCTAATGTGTTAAAATACTTTATGCTGGTAATAACAC |
| | | TAAGAGTAGGGCACTAGAAATTTTAAGTGAAGATAATGTGTTGCA |
| | | GTTACTGCACTCAATGGCTTACTATTATAAACCAAAACTGGGATC |
| | | ACTAAGCTCCAGTCAGTCAAAATGATCAAAATTATTGAAGAGAAT |
| | | AAGCAATTCTGTTCTTTATTAGGACACAGTAGATACAGACTACAA |
| | | AGTGGAGTGTGCTTAATAAGAGGTAGCATTTGTTAAGTGTCAATT |
| | | ACTCTATTATCCCTTGGAGCTTCTCAAAATAACCATATAAGGTGT |
| | | AAGATGTTAAAGGTTATGGTTACACTCAGTGCACAGGTAAGCTAA |
| | | TAGGCTGAGAGAAGCTAAATTACTTACTGGGGTCTCACAGTAAGA |
| | | AAGTGAGCTGAAGTTTCAGCCCAGATTTAACTGGATTCTGGGCTC |
| | | TTTATTCATGTTACTTCATGAATCTGTTTCTCAATTGTGCAGAAAA |
| | | AAGGGGGCTATTTATAAGAAAAGCAATAAACAAACAAGTAATGA |
| | | TCTCAAATAAGTAATGCAAGAAATAGTGAGATTTCAAAATCAGTG |
| | | GCAGCGATTTCTCAGTTCTGTCCTAAGTGGCCTTGCTCAATCACCT |
| | | GCTATCTTTTAGTGGAGCTTTGAAATTATGTTTCAGACAACTTCGA |
| | | TTCAGTTCTAGAATGTTTGACTCAGCAAATTCACAGGCTCATCTTT |
| | | CTAACTTGATGGTGAATATGGAAATTCAGCTAAATGGATGTTAAT |
| | | AAAATTCAAACGTTTTAAGGACAGATGGAAATGACAGAATTTTA |
| | | AGGTAAAATATATGAAGGAATATAAGATAAAGGATTTTTCTACCT |
| | | TCAGCAAAAACATACCCACTAATTAGTAAAATTAATAGGCGAAA |
| | | AAAAGTTGCATGCTCTTATACTGTAATGATTATCATTTTAAAACTA |
| | | GCTTTTTGCCTTCGAGCTATCGGGGTAAAGACCTACAGGAAAACT |
| | | ACTGTCGAAATCCTCGAGGGGAAGAAGGGGGACCCTGGTGTTTC |
| | | ACAAGCAATCCAGAGGTACGCTACGAAGTCTGTGACATTCCTCAG |
| | | TGTTCAGAAGTTGAATGCATGACCTGCAATGGGGAGAGTTATCGA |
| | | GGTCTCATGGATCATACAGAATCAGGCAAGATTTGTCAGCGCTGG |
| | | GATCATCAGACACCACACCGGCACAAATTCTTGCCTGAAAGATAT |
| | | CCCGACAAGGGCTTTGATGATAATTATTGCCGCAATCCCGATGGC |
| | | CAGCCGAGGCCATGGTGCTATACTCTTGACCCTCACACCCGCTGG |
| | | GAGTACTGTGCAATTAAAACATGCGCTGACAATACTATGAATGAC |
| | | ACTGATGTTCCTTTGGAAACAACTGAATGCATCCAAGGTCAAGGA |
| | | GAAGGCTACAGGGGCACTGTCAATACCATTTGGAATGGAATTCCA |
| | | TGTCAGCGTTGGGATTCTCAGTATCCTCACGAGCATGACATGACT |
| | | CCTGAAAATTTCAAGTGCAAGGACCTACGAGAAAATTACTGCCG |
| | | AAATCCAGATGGGTCTGAATCACCCTGGTGTTTTACCACTGATCC |
| | | AAACATCCGAGTTGGCTACTGCTCCCAAATTCCAAACTGTGATAT |
| | | GTCACATGGACAAGATTGTTATCGTGGGAATGGCAAAAATTATAT |
| | | GGGCAACTTATCCCAAACAAGATCTGGACTAACATGTTCAATGTG |
| | | GGACAAGAACATGGAAGACTTACATCGTCATATCTTCTGGGAACC |
| | | AGATGCAAGTAAGCTGAATGAGAATTACTGCCGAAATCCAGATG |
| | | ATGATGCTCATGGACCCTGGTGCTACACGGGAAATCCACTCATTC |
| | | CTTGGGATTATTGCCCTATTTCTCGTTGTGAAGGTGATACCACACC |
| | | TACAATAGTCAATTTAGACCATCCCGTAATATCTTGTGCCAAAAC |
| | | GAAACAATTGCGAGTTGTAAATGGGATTCCAACACGAACAAACA |
| | | TAGGATGGATGGTTAGTTTGAGATACAGAAATAAACATATCTGCG |
| | | GAGGATCATTGATAAAGGAGAGTTGGGTTCTTACTGCACGACAGT |
| | | GTTTCCCTTCTCGAGACTTGAAAGATTATGAAGCTTGGCTTGGAA |
| | | TTCATGATGTCCACGGAAGAGGAGATGAGAAATGCAAACAGGTT |
| | | CTCAATGTTTCCCAGCTGGTATATGGCCCTGAAGGATCAGATCTG |
| | | GTTTTAATGAAGCTTGCCAGGCCTGCTGTCCTGGATGATTTTGTTA |
| | | GTACGATTGATTTACCTAATTATGGATGCACAATTCCTGAAAAGA |
| | | CCAGTTGCAGTGTTTATGGCTGGGGCTACACTGGATTGATCAACT |
| | | ATGATGGCCTATTACGAGTGGCACATCTCTATATAATGGGAAATG |
| | | AGAAATGCAGCCAGCATCATCGAGGGAAGGTGACTCTGAATGAG |
| | | TCTGAAATATGTGCTGGGGCTGAAAAGATTGGATCAGGACCATGT |
| | | GAGGGGGATTATGGTGGCCCACTTGTTTGTGAGCAACATAAAATG |
| | | AGAATGGTTCTTGGTGTCATTGTTCCTGGTCGTGGATGTGCCATTC |
| | | CAAATCGTCCTGGTATTTTTGTCCGAGTAGCATATTATGCAAAAT |
| | | GGATACACAAAATTATTTTAACATATAAGGTACCACAGTCATAG |
| SEQ ID | Nucleotide | ATGTGGGTGACCAAACTCCTGCCAGCCCTGCTGCTGCAGCATGTC |
| NO. 32 | sequence of | CTCCTGCATCTCCTCCTGCTCCCCATCGCCATCCCCTATGCAGAGG |
| | HGF-X8 | GACAAAGGAAAAGAAGAAATACAATTCATGAATTCAAAAAATCA |
| | | GCAAAGACTACCCTAATCAAAATAGATCCAGCACTGAAGATAAA |
| | | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGGTAAGAACAG |
| | | TATGAAGAAAAGAGATGAAGCCTCTGTCTTTTTTACATGTTAACA |
| | | GTCTCATATTAGTCCTTCAGAATAATTCTACAATCCTAAAATAACT |
| | | TAGCCAACTTGCTGAATTGTATTACGGCAAGGTTTATATGAATTC |
| | | ATGACTGATATTTAGCAAATGATTAATTAATATGTTAATAAAATG |
| | | TAGCCAAAACAATATCTTACCTTAATGCCTCAATTTGTAGATCTC |
| | | GGTATTTGTGGATCCTTATGTTTCAGACAACTTCGATTCAGTTCTA |
| | | GAATGTTTGACTCAGCAAATTCACAGGCTCATCTTTCTAACTTGAT |
| | | GGTGAATATGGAAATTCAGCTAAATGGATGTTAATAAAATTCAAA |
| | | CGTTTTAAGGACAGATGGAAATGACAGAATTTTAAGGTAAAATAT |
| | | ATGAAGGAATATAAGATAAAGGATTTTTCTACCTTCAGCAAAAAC |
| | | ATACCCACTAATTAGTAAAATTAATAGGCGAAAAAAAGTTGCAT |
| | | GCTCTTATACTGTAATGATTATCATTTTAAAACTAGCTTTTTGCCT |
| | | TCGAGCTATCGGGGTAAAGACCTACAGGAAAACTACTGTCGAAA |
| | | TCCTCGAGGGGAAGAAGGGGGACCCTGGTGTTTCACAAGCAATC |
| | | CAGAGGTACGCTACGAAGTCTGTGACATTCCTCAGTGTTCAGAAG |
| | | TTGAATGCATGACCTGCAATGGGGAGAGTTATCGAGGTCTCATGG |
| | | ATCATACAGAATCAGGCAAGATTTGTCAGCGCTGGGATCATCAGA |
| | | CACCACACCGGCACAAATTCTTGCCTGAAAGATATCCCGACAAGG |
| | | GCTTTGATGATAATTATTGCCGCAATCCCGATGGCCAGCCGAGGC |
| | | CATGGTGCTATACTCTTGACCCTCACACCCGCTGGGAGTACTGTG |
| | | CAATTAAAACATGCGCTGACAATACTATGAATGACACTGATGTTC |
| | | CTTTGGAAACAACTGAATGCATCCAAGGTCAAGGAGAAGGCTAC |
| | | AGGGGCACTGTCAATACCATTTGGAATGGAATTCCATGTCAGCGT |
| | | TGGGATTCTCAGTATCCTCACGAGCATGACATGACTCCTGAAAAT |
| | | TTCAAGTGCAAGGACCTACGAGAAAATTACTGCCGAAATCCAGA |
| | | TGGGTCTGAATCACCCTGGTGTTTTACCACTGATCCAAACATCCG |
| | | AGTTGGCTACTGCTCCCAAATTCCAAACTGTGATATGTCACATGG |
| | | ACAAGATTGTTATCGTGGGAATGGCAAAAATTATATGGGCAACTT |
| | | ATCCCAAACAAGATCTGGACTAACATGTTCAATGTGGGACAAGA |
| | | ACATGGAAGACTTACATCGTCATATCTTCTGGGAACCAGATGCAA |
| | | GTAAGCTGAATGAGAATTACTGCCGAAATCCAGATGATGATGCTC |
| | | ATGGACCCTGGTGCTACACGGGAAATCCACTCATTCCTTGGGATT |
| | | ATTGCCCTATTTCTCGTTGTGAAGGTGATACCACACCTACAATAG |
| | | TCAATTTAGACCATCCCGTAATATCTTGTGCCAAAACGAAACAAT |
| | | TGCGAGTTGTAAATGGGATTCCAACACGAACAAACATAGGATGG |
| | | ATGGTTAGTTTGAGATACAGAAATAAACATATCTGCGGAGGATCA |
| | | TTGATAAAGGAGAGTTGGGTTCTTACTGCACGACAGTGTTTCCCT |
| | | TCTCGAGACTTGAAAGATTATGAAGCTTGGCTTGGAATTCATGAT |
| | | GTCCACGGAAGAGGAGATGAGAAATGCAAACAGGTTCTCAATGT |
| | | TTCCCAGCTGGTATATGGCCCTGAAGGATCAGATCTGGTTTTAAT |
| | | GAAGCTTGCCAGGCCTGCTGTCCTGGATGATTTTGTTAGTACGAT |
| | | TGATTTACCTAATTATGGATGCACAATTCCTGAAAAGACCAGTTG |
| | | CAGTGTTTATGGCTGGGGCTACACTGGATTGATCAACTATGATGG |
| | | CCTATTACGAGTGGCACATCTCTATATAATGGGAAATGAGAAATG |
| | | CAGCCAGCATCATCGAGGGAAGGTGACTCTGAATGAGTCTGAAA |
| | | TATGTGCTGGGGCTGAAAAGATTGGATCAGGACCATGTGAGGGG |
| | | GATTATGGTGGCCCACTTGTTTGTGAGCAACATAAAATGAGAATG |
| | | GTTCTTGGTGTCATTGTTCCTGGTCGTGGATGTGCCATTCCAAATC |
| | | GTCCTGGTATTTTTGTCCGAGTAGCATATTATGCAAAATGGATAC |
| | | ACAAAATTATTTTAACATATAAGGTACCACAGTCATAG |
| SEQ ID | sequence of | ATGTGGGTGACCAAACTCCTGCCAGCCCTGCTGCTGCAGCATGTC |
| NO. 33 | polynucleotide | CTCCTGCATCTCCTCCTGCTCCCCATCGCCATCCCCTATGCAGAGG |
| | sequence | GACAAAGGAAAAGAAGAAATACAATTCATGAATTCAAAAAATCA |
| | encoding | GCAAAGACTACCCTAATCAAAATAGATCCAGCACTGAAGATAAA |
| | HGF728. | AACCAAAAAAGTGAATACTGCAGACCAATGTGCTAATAGATGTA |
| | | CTAGGAATAAAGGACTTCCATTCACTTGCAAGGCTTTTGTTTTTGA |
| | | TAAAGCAAGAAAACAATGCCTCTGGTTCCCCTTCAATAGCATGTC |
| | | AAGTGGAGTGAAAAAAGAATTTGGCCATGAATTTGACCTCTATGA |
| | | AAACAAAGACTACATTAGAAACTGCATCATTGGTAAAGGACGCA |
| | | GCTACAAGGGAACAGTATCTATCACTAAGAGTGGCATCAAATGTC |
| | | AGCCCTGGAGTTCCATGATACCACACGAACACAGCTTTTTGCCTT |
| | | CGAGCTATCGGGGTAAAGACCTACAGGAAAACTACTGTCGAAAT |
| | | CCTCGAGGGGAAGAAGGGGGACCCTGGTGTTTCACAAGCAATCC |
| | | AGAGGTACGCTACGAAGTCTGTGACATTCCTCAGTGTTCAGAAGT |
| | | TGAATGCATGACCTGCAATGGGGAGAGTTATCGAGGTCTCATGGA |
| | | TCATACAGAATCAGGCAAGATTTGTCAGCGCTGGGATCATCAGAC |
| | | ACCACACCGGCACAAATTCTTGCCTGAAAGATATCCCGACAAGG |
| | | GCTTTGATGATAATTATTGCCGCAATCCCGATGGCCAGCCGAGGC |
| | | CATGGTGCTATACTCTTGACCCTCACACCCGCTGGGAGTACTGTG |
| | | CAATTAAAACATGCGCTGACAATACTATGAATGACACTGATGTTC |
| | | CTTTGGAAACAACTGAATGCATCCAAGGTCAAGGAGAAGGCTAC |
| | | AGGGGCACTGTCAATACCATTTGGAATGGAATTCCATGTCAGCGT |
| | | TGGGATTCTCAGTATCCTCACGAGCATGACATGACTCCTGAAAAT |
| | | TTCAAGTGCAAGGACCTACGAGAAAATTACTGCCGAAATCCAGA |
| | | TGGGTCTGAATCACCCTGGTGTTTTACCACTGATCCAAACATCCG |
| | | AGTTGGCTACTGCTCCCAAATTCCAAACTGTGATATGTCACATGG |
| | | ACAAGATTGTTATCGTGGGAATGGCAAAAATTATATGGGCAACTT |
| | | ATCCCAAACAAGATCTGGACTAACATGTTCAATGTGGGACAAGA |
| | | ACATGGAAGACTTACATCGTCATATCTTCTGGGAACCAGATGCAA |
| | | GTAAGCTGAATGAGAATTACTGCCGAAATCCAGATGATGATGCTC |
| | | ATGGACCCTGGTGCTACACGGGAAATCCACTCATTCCTTGGGATT |
| | | ATTGCCCTATTTCTCGTTGTGAAGGTGATACCACACCTACAATAG |
| | | TCAATTTAGACCATCCCGTAATATCTTGTGCCAAAACGAAACAAT |
| | | TGCGAGTTGTAAATGGGATTCCAACACGAACAAACATAGGATGG |
| | | ATGGTTAGTTTGAGATACAGAAATAAACATATCTGCGGAGGATCA |
| | | TTGATAAAGGAGAGTTGGGTTCTTACTGCACGACAGTGTTTCCCT |
| | | TCTCGAGACTTGAAAGATTATGAAGCTTGGCTTGGAATTCATGAT |
| | | GTCCACGGAAGAGGAGATGAGAAATGCAAACAGGTTCTCAATGT |
| | | TTCCCAGCTGGTATATGGCCCTGAAGGATCAGATCTGGTTTTAAT |
| | | GAAGCTTGCCAGGCCTGCTGTCCTGGATGATTTTGTTAGTACGAT |
| | | TGATTTACCTAATTATGGATGCACAATTCCTGAAAAGACCAGTTG |
| | | CAGTGTTTATGGCTGGGGCTACACTGGATTGATCAACTATGATGG |
| | | CCTATTACGAGTGGCACATCTCTATATAATGGGAAATGAGAAATG |
| | | CAGCCAGCATCATCGAGGGAAGGTGACTCTGAATGAGTCTGAAA |
| | | TATGTGCTGGGGCTGAAAAGATTGGATCAGGACCATGTGAGGGG |
| | | GATTATGGTGGCCCACTTGTTTGTGAGCAACATAAAATGAGAATG |
| | | GTTCTTGGTGTCATTGTTCCTGGTCGTGGATGTGCCATTCCAAATC |
| | | GTCCTGGTATTTTTGTCCGAGTAGCATATTATGCAAAATGGATAC |
| | | ACAAAATTATTTTAACATATAAGGTACCACAGTCATAG |
| SEQ ID | F primer of | AGCTGGCAATTCCGGTTCGCTTGCTGCGTCAGACCCCGTA |
| NO. 34 | the first | |
| | primer pair | |
| | (pTx) | |
| SEQ ID | R primer of | TACGGGGTCTGACGCAGCAAGCGAACCGGAATTGCCAGCT |
| NO. 35 | the first | |
| | primer pair | |
| | (pTx) | |
| SEQ ID | F primer of | CTAATCCATAACATGGCTCTAGACTTAAGGCAGCGGCAGA |
| NO. 36 | the second | |
| | primer pair | |
| | (pTx) | |
| SEQ ID | R primer of | TCTGCCGCTGCCTTAAGTCTAGAGCCATGTTATGGATTAG |
| NO. 37 | the second | |
| | primer pair | |
| | (pTx) | |
| SEQ ID | pTx | CAGGGTCGGAACAGGAGAGCGCACGAGGGAGCTTCCAGGGGGA |
| NO. 38 | | AACGCCTGGTATCTTTATAGTCCTGTCGGGTTTCGCCACCTCTGAC |
| | | TTGAGCGTCGATTTTTGTGATGCTCGTCAGGGGGGCGGAGCCTAT |
| | | GGAAAAACGCCAGCAACGCGGCCTTTTTACGGTTCCTGGCCTTTT |
| | | GCTGGCCTTTTGCTCACATGCGCGTTGACATTGATTATTGACTAGT |
| | | TATTAATAGTAATCAATTACGGGGTCATTAGTTCATAGCCCATAT |
| | | ATGGAGTTCCGCGTTACATAACTTACGGTAAATGGCCCGCCTGGC |
| | | TGACCGCCCAACGACCCCCGCCCATTGACGTCAATAATGACGTAT |
| | | GTTCCCATAGTAACGCCAATAGGGACTTTCCATTGACGTCAATGG |
| | | GTGGAGTATTTACGGTAAACTGCCCACTTGGCAGTACATCAAGTG |
| | | TATCATATGCCAAGTCCGCCCCCTATTGACGTCAATGACGGTAAA |
| | | TGGCCCGCCTGGCATTATGCCCAGTACATGACCTTACGGGACTTT |
| | | CCTACTTGGCAGTACATCTACGTATTAGTCATCGCTATTACCATGG |
| | | TGATGCGGTTTTGGCAGTACACCAATGGGCGTGGATAGCGGTTTG |
| | | ACTCACGGGGATTTCCAAGTCTCCACCCCATTGACGTCAATGGGA |
| | | GTTTGTTTTGGCACCAAAATCAACGGGACTTTCCAAAATGTCGTA |
| | | ATAACCCCGCCCCGTTGACGCAAATGGGCGGTAGGCGTGTACGGT |
| | | GGGAGGTCTATATAAGCAGAGCTCGTTTAGTGAACCGTCAGATCG |
| | | CCTGGAGACGCCATCCACGCTGTTTTGACCTCCATAGAAGACACC |
| | | GGGACCGATCCAGCCTCCGCGGCCGGGAACGGTGCATTGGAACG |
| | | CGGATTCCCCGTGCCAAGAGTGACGTAAGTACCGCCTATAGACTC |
| | | TATAGGCACACCCCTTTGGCTCTTATGCATGCTATACTGTTTTTGG |
| | | CTTGGGGCCTATACACCCCCGCTTCCTTATGCTATAGGTGATGGT |
| | | ATAGCTTAGCCTATAGGTGTGGGTTATTGACCATTATTGACCACT |
| | | CCCCTATTGGTGACGATACTTTCCATTACTAATCCATAACATGGCT |
| | | CTAGACTTAAGGCAGCGGCAGAAGAAGATGTAGGCAGCTGAGTT |
| | | GTTGTATTCTGATAAGAGTCAGAGGTAACTCCCGTTGCGGTGCTG |
| | | TTAACGGTGGAGGGCAGTGTAGTCTGAGCAGTACTCGTTGCTGCC |
| | | GCGCGCGCCACCAGACATAATAGCTGACAGACTAACAGACTGTT |
| | | CCTTTCCATGGGTCTTTTCTGCAGTCACCGTCCTTGACACGAAGCT |
| | | TATCGATGTCGACCTCGAGTCTAGAGGGCCCGTTTAAACCCGCTG |
| | | ATCAGCCTCGACTGTGCCTTCTAGTTGCCAGCCATCTGTTGTTTGC |
| | | CCCTCCCCCGTGCCTTCCTTGACCCTGGAAGGTGCCACTCCCACTG |
| | | TCCTTTCCTAATAAAATGAGGAAATTGCATCGCATTGTCTGAGTA |
| | | GGTGTCATTCTATTCTGGGGGGTGGGGTGGGGCAGGACAGCAAG |
| | | GGGGAGGATTGGGAAGACAATAGCAGGCATGCTGGGGAGTCGAA |
| | | ATTCAGAAGAACTCGTCAAGAAGGCGATAGAAGGCGATGCGCTG |
| | | CGAATCGGGAGCGGCGATACCGTAAAGCACGAGGAAGCGGTCAG |
| | | CCCATTCGCCGCCAAGCTCTTCAGCAATATCACGGGTAGCCAACG |
| | | CTATGTCCTGATAGCGGTCCGCCACACCCAGCCGGCCACAGTCGA |
| | | TGAATCCAGAAAAGCGGCCATTTTCCACCATGATATTCGGCAAGC |
| | | AGGCATCGCCATGGGTCACGACGAGATCCTCGCCGTCGGGCATGC |
| | | TCGCCTTGAGCCTGGCGAACAGTTCGGCTGGCGCGAGCCCCTGAT |
| | | GCTCTTCGTCCAGATCATCCTGATCGACAAGACCGGCTTCCATCC |
| | | GAGTACGTGCTCGCTCGATGCGATGTTTCGCTTGGTGGTCGAATG |
| | | GGCAGGTAGCCGGATCAAGCGTATGCAGCCGCCGCATTGCATCA |
| | | GCCATGATGGATACTTTCTCGGCAGGAGCAAGGTGAGATGACAG |
| | | GAGATCCTGCCCCGGCACTTCGCCCAATAGCAGCCAGTCCCTTCC |
| | | CGCTTCAGTGACAACGTCGAGCACAGCTGCGCAAGGAACGCCCG |
| | | TCGTGGCCAGCCACGATAGCCGCGCTGCCTCGTCTTGCAGTTCAT |
| | | TCAGGGCACCGGACAGGTCGGTCTTGACAAAAAGAACCGGGCGC |
| | | CCCTGCGCTGACAGCCGGAACACGGCGGCATCAGAGCAGCCGAT |
| | | TGTCTGTTGTGCCCAGTCATAGCCGAATAGCCTCTCCACCCAAGC |
| | | GGCCGGAGAACCTGCGTGCAATCCATCTTGTTCAATCATGCGAAA |
| | | CGATCCTCATCCTGTCTCTTGATCAGATCTTGATCCCCTGCGCCAT |
| | | CAGATCCTTGGCGGCAAGAAAGCCATCCAGTTTACTTTGCAGGGC |
| | | TTCCCAACCTTACCAGAGGGCGCCCCAGCTGGCAATTCCGGTTCG |
| | | CTTGCTGCGTCAGACCCCGTAGAAAAGATCAAAGGATCTTCTTGA |
| | | GATCCTTTTTTTCTGCGCGTAATCTGCTGCTTGCAAACAAAAAAA |
| | | CCACCGCTACCAGCGGTGGTTTGTTTGCCGGATCAAGAGCTACCA |
| | | ACTCTTTTTCCGAAGGTAACTGGCTTCAGCAGAGCGCAGATACCA |
| | | AATACTGTTCTTCTAGTGTAGCCGTAGTTAGGCCACCACTTCAAG |
| | | AACTCTGTAGCACCGCCTACATACCTCGCTCTGCTAATCCTGTTAC |
| | | CAGTGGCTGCTGCCAGTGGCGATAAGTCGTGTCTTACCGGGTTGG |
| | | ACTCAAGACGATAGTTACCGGATAAGGCGCAGCGGTCGGGCTGA |
| | | ACGGGGGGTTCGTGCACACAGCCCAGCTTGGAGCGAACGACCTA |
| | | CACCGAACTGAGATACCTACAGCGTGAGCTATGAGAAAGCGCCA |
| | | CGCTTCCCGAAGGGAGAAAGGCGGACAGGTATCCGGTAAGCGGC |
| | | AGGGTCGGAACAGGAGAGCGCACGAGGGAGCTTCCAGGGGGAA |
| | | ACGCCTGGTATCTTTATAGTCCTGTCGGGTTTCGCCACCTCTGACT |
| | | TGAGCGTCGATTTTTGTGATGCTCGTCAGGGGGGCGGAGCCTATG |
| | | GAAAAACGCCAGCAACGCGGCCTTTTTACGGTTCCTGGCCTTTTG |
| | | CTGGCCTTTTGCTCACATG |
| SEQ ID | pTx-IGF- | CAGGGTCGGAACAGGAGAGCGCACGAGGGAGCTTCCAGGGGGA |
| NO. 39 | 1X10 | AACGCCTGGTATCTTTATAGTCCTGTCGGGTTTCGCCACCTCTGAC |
| | | TTGAGCGTCGATTTTTGTGATGCTCGTCAGGGGGGCGGAGCCTAT |
| | | GGAAAAACGCCAGCAACGCGGCCTTTTTACGGTTCCTGGCCTTTT |
| | | GCTGGCCTTTTGCTCACATGCGCGTTGACATTGATTATTGACTAGT |
| | | TATTAATAGTAATCAATTACGGGGTCATTAGTTCATAGCCCATAT |
| | | ATGGAGTTCCGCGTTACATAACTTACGGTAAATGGCCCGCCTGGC |
| | | TGACCGCCCAACGACCCCCGCCCATTGACGTCAATAATGACGTAT |
| | | GTTCCCATAGTAACGCCAATAGGGACTTTCCATTGACGTCAATGG |
| | | GTGGAGTATTTACGGTAAACTGCCCACTTGGCAGTACATCAAGTG |
| | | TATCATATGCCAAGTCCGCCCCCTATTGACGTCAATGACGGTAAA |
| | | TGGCCCGCCTGGCATTATGCCCAGTACATGACCTTACGGGACTTT |
| | | CCTACTTGGCAGTACATCTACGTATTAGTCATCGCTATTACCATGG |
| | | TGATGCGGTTTTGGCAGTACACCAATGGGCGTGGATAGCGGTTTG |
| | | ACTCACGGGGATTTCCAAGTCTCCACCCCATTGACGTCAATGGGA |
| | | GTTTGTTTTGGCACCAAAATCAACGGGACTTTCCAAAATGTCGTA |
| | | ATAACCCCGCCCCGTTGACGCAAATGGGCGGTAGGCGTGTACGGT |
| | | GGGAGGTCTATATAAGCAGAGCTCGTTTAGTGAACCGTCAGATCG |
| | | CCTGGAGACGCCATCCACGCTGTTTTGACCTCCATAGAAGACACC |
| | | GGGACCGATCCAGCCTCCGCGGCCGGGAACGGTGCATTGGAACG |
| | | CGGATTCCCCGTGCCAAGAGTGACGTAAGTACCGCCTATAGACTC |
| | | TATAGGCACACCCCTTTGGCTCTTATGCATGCTATACTGTTTTTGG |
| | | CTTGGGGCCTATACACCCCCGCTTCCTTATGCTATAGGTGATGGT |
| | | ATAGCTTAGCCTATAGGTGTGGGTTATTGACCATTATTGACCACT |
| | | CCCCTATTGGTGACGATACTTTCCATTACTAATCCATAACATGGCT |
| | | CTAGACTTAAGGCAGCGGCAGAAGAAGATGTAGGCAGCTGAGTT |
| | | GTTGTATTCTGATAAGAGTCAGAGGTAACTCCCGTTGCGGTGCTG |
| | | TTAACGGTGGAGGGCAGTGTAGTCTGAGCAGTACTCGTTGCTGCC |
| | | GCGCGCGCCACCAGACATAATAGCTGACAGACTAACAGACTGTT |
| | | CCTTTCCATGGGTCTTTTCTGCAGTCACCGTCCTTGACACGAAGCT |
| | | TATCGATATGGGAAAAATCAGCAGTCTTCCAACCCAATTATTTAA |
| | | GTGCTGCTTTTGTGATTTCTTGAAGGTGAAGATGCACACCATGTC |
| | | CTCCTCGCATCTCTTCTACCTGGCGCTGTGCCTGCTCACCTTCACC |
| | | AGCTCTGCCACGGCTGGACCGGAGACGCTCTGCGGGGCTGAGCT |
| | | GGTGGATGCTCTTCAGTTCGTGTGTGGAGACAGGGGCTTTTATTT |
| | | CAACAAGCCCACAGGGTATGGCTCCAGCAGTCGGAGGGCGCCTC |
| | | AGACAGGCATCGTGGATGAGTGCTGCTTCCGGAGCTGTGATCTAA |
| | | GGAGGCTGGAGATGTATTGCGCACCCCTCAAGCCTGCCAAGTCAG |
| | | CTCGCTCTGTCCGTGCCCAGCGCCACACCGACATGCCCAAGACCC |
| | | AGAAGGTAAGCCCACCTGGGTGGGATCCAGCCATCCTCAAGTGG |
| | | TCTCTCTCTTGTGCATGTGGGTGGGCCAAGCAGAAATCCTGCCCC |
| | | ATAGTCTCCTGGCTTACAAGTCAGAAAAGCTCCTTTGCACCAAAG |
| | | GGATGGATTACATCCCCATCTCTTTGCTAAACAAACATGGGCTTT |
| | | GGTGTCAGACAAAAGTGAAGTCCTGGCTTTCTCACACACCAGCTT |
| | | AGAGAGAAAAGACTTTTAGGTGAATGTGGCAGGAAAGCGTGCTT |
| | | GCTGGGGCAAAGGCAGATTCATTCTTTCTCTTCCCAGTATCAGCC |
| | | CCCATCTACCAACAAGAACACGAAGTCTCAGAGAAGGAAAGGAA |
| | | GTACATTTGAAGAACGCAAGTAGCTTTTTCTCCTTTATTTATAGGA |
| | | AGTACATTTGAAGAACGCAAGTAGAGGGAGTGCAGGAAACAAGA |
| | | ACTACAGGATGTAGGTCGACCTCGAGTCTAGAGGGCCCGTTTAAA |
| | | CCCGCTGATCAGCCTCGACTGTGCCTTCTAGTTGCCAGCCATCTGT |
| | | TGTTTGCCCCTCCCCCGTGCCTTCCTTGACCCTGGAAGGTGCCACT |
| | | CCCACTGTCCTTTCCTAATAAAATGAGGAAATTGCATCGCATTGT |
| | | CTGAGTAGGTGTCATTCTATTCTGGGGGGTGGGGTGGGGCAGGAC |
| | | AGCAAGGGGGAGGATTGGGAAGACAATAGCAGGCATGCTGGGG |
| | | AGTCGAAATTCAGAAGAACTCGTCAAGAAGGCGATAGAAGGCGA |
| | | TGCGCTGCGAATCGGGAGCGGCGATACCGTAAAGCACGAGGAAG |
| | | CGGTCAGCCCATTCGCCGCCAAGCTCTTCAGCAATATCACGGGTA |
| | | GCCAACGCTATGTCCTGATAGCGGTCCGCCACACCCAGCCGGCCA |
| | | CAGTCGATGAATCCAGAAAAGCGGCCATTTTCCACCATGATATTC |
| | | GGCAAGCAGGCATCGCCATGGGTCACGACGAGATCCTCGCCGTC |
| | | GGGCATGCTCGCCTTGAGCCTGGCGAACAGTTCGGCTGGCGCGAG |
| | | CCCCTGATGCTCTTCGTCCAGATCATCCTGATCGACAAGACCGGC |
| | | TTCCATCCGAGTACGTGCTCGCTCGATGCGATGTTTCGCTTGGTGG |
| | | TCGAATGGGCAGGTAGCCGGATCAAGCGTATGCAGCCGCCGCAT |
| | | TGCATCAGCCATGATGGATACTTTCTCGGCAGGAGCAAGGTGAGA |
| | | TGACAGGAGATCCTGCCCCGGCACTTCGCCCAATAGCAGCCAGTC |
| | | CCTTCCCGCTTCAGTGACAACGTCGAGCACAGCTGCGCAAGGAAC |
| | | GCCCGTCGTGGCCAGCCACGATAGCCGCGCTGCCTCGTCTTGCAG |
| | | TTCATTCAGGGCACCGGACAGGTCGGTCTTGACAAAAAGAACCG |
| | | GGCGCCCCTGCGCTGACAGCCGGAACACGGCGGCATCAGAGCAG |
| | | CCGATTGTCTGTTGTGCCCAGTCATAGCCGAATAGCCTCTCCACC |
| | | CAAGCGGCCGGAGAACCTGCGTGCAATCCATCTTGTTCAATCATG |
| | | CGAAACGATCCTCATCCTGTCTCTTGATCAGATCTTGATCCCCTGC |
| | | GCCATCAGATCCTTGGCGGCAAGAAAGCCATCCAGTTTACTTTGC |
| | | AGGGCTTCCCAACCTTACCAGAGGGCGCCCCAGCTGGCAATTCCG |
| | | GTTCGCTTGCTGCGTCAGACCCCGTAGAAAAGATCAAAGGATCTT |
| | | CTTGAGATCCTTTTTTTCTGCGCGTAATCTGCTGCTTGCAAACAAA |
| | | AAAACCACCGCTACCAGCGGTGGTTTGTTTGCCGGATCAAGAGCT |
| | | ACCAACTCTTTTTCCGAAGGTAACTGGCTTCAGCAGAGCGCAGAT |
| | | ACCAAATACTGTTCTTCTAGTGTAGCCGTAGTTAGGCCACCACTT |
| | | CAAGAACTCTGTAGCACCGCCTACATACCTCGCTCTGCTAATCCT |
| | | GTTACCAGTGGCTGCTGCCAGTGGCGATAAGTCGTGTCTTACCGG |
| | | GTTGGACTCAAGACGATAGTTACCGGATAAGGCGCAGCGGTCGG |
| | | GCTGAACGGGGGGTTCGTGCACACAGCCCAGCTTGGAGCGAACG |
| | | ACCTACACCGAACTGAGATACCTACAGCGTGAGCTATGAGAAAG |
| | | CGCCACGCTTCCCGAAGGGAGAAAGGCGGACAGGTATCCGGTAA |
| | | GCGGCAGGGTCGGAACAGGAGAGCGCACGAGGGAGCTTCCAGGG |
| | | GGAAACGCCTGGTATCTTTATAGTCCTGTCGGGTTTCGCCACCTCT |
| | | GACTTGAGCGTCGATTTTTGTGATGCTCGTCAGGGGGGCGGAGCC |
| | | TATGGAAAAACGCCAGCAACGCGGCCTTTTTACGGTTCCTGGCCT |
| | | TTTGCTGGCCTTTTGCTCACATG |

1. A method of treating neuropathy, comprising the steps of:
administering to a subject having neuropathy a therapeutically effective amount of an IGF-1-encoding DNA construct comprising a polynucleotide of SEQ ID NO: 10; and
administering to the subject a therapeutically effective amount of an HGF-encoding DNA construct comprising a polynucleotide of SEQ ID NO: 13.

2-20. (canceled)

21. The method of claim 1, wherein the IGF-1-encoding DNA construct and the HGF-encoding DNA construct are administered in an amount sufficient to reduce pain in the subject.

22. The method of claim 1, wherein the subject has diabetic neuropathy.

23. The method of claim 1, wherein the IGF-1-encoding DNA construct and the HGF-encoding DNA construct are administered by a plurality of intramuscular injections.

24-29. (canceled)

30. The method of claim 1, wherein the IGF-1-encoding DNA construct and the HGF-encoding DNA construct are co-administered.

31. The method of claim 30, wherein the IGF-1-encoding DNA construct and the HGF-encoding DNA construct are co-administered by an intramuscular injection.

32. The method of claim 1, wherein the step of administering the IGF-1-encoding DNA construct and the step of administering the HGF-encoding DNA construct are performed separately.

33. The method of claim 32, wherein the step of administering the IGF-1-encoding DNA construct and the step of administering the HGF-encoding DNA construct are performed at least three weeks apart.

34-37. (canceled)

38. A pharmaceutical composition comprising:
an IGF-1-encoding DNA construct comprising a polynucleotide of SEQ ID NO: 10;
an HGF-encoding DNA construct comprising a polynucleotide of SEQ ID NO: 13, and
a pharmaceutically acceptable excipient.

39-45. (canceled)

46. The pharmaceutical composition of claim 38, wherein the IGF-1-encoding DNA construct further comprises a plasmid vector.

47. The pharmaceutical composition of claim 46, wherein the plasmid vector is pCK.

48. The pharmaceutical composition of claim 47, wherein the IGF-1-encoding DNA construct is pCK-IGF-1X10.

49. The pharmaceutical composition of claim 46, wherein the plasmid vector is pTx.

50. The pharmaceutical composition of claim 49, wherein the IGF-1-encoding DNA construct is IGF-1X10.

51-62. (canceled)

63. A kit for treating neuropathy, comprising:
a first pharmaceutical composition comprising an IGF-1-encoding DNA construct comprising a polynucleotide of SEQ ID NO: 10, and a first pharmaceutically acceptable excipient; and
a second pharmaceutical composition comprising an HGF-encoding DNA construct comprising a polynucleotide of SEQ ID NO: 13, and a second pharmaceutically acceptable excipient.

64-121. (canceled)


SEQUENCE LISTING (41 sequences)

SEQ ID NO: 1 - DNA, 402 bp, Homo sapiens
  source 1..402: mol_type = unassigned DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaag

SEQ ID NO: 2 - DNA, 1505 bp, Homo sapiens
  source 1..1505: mol_type = unassigned DNA
gtaagcccacctgggtgggatccagccatcctcaagtggtctctctcttgtgcatgtgggtgggccaagcagaaatcctgccccatagtctcctggcttacaagtcagaaaagctcctttgcaccaaagggatggattacatccccatctctttggtcactctgcattgcaaatttcccctcccaccgctatggacgatgtgatgattggaagatgttacaaaacagtggctaaacaaacatgggctttggtgtcagacaaaagtgaagtcctggctttctcacacaccagcttagagcccttggcaaataatgtgatgtacccaagcctcagtttcatcagtaacattgggataataataatatctaccacatcagtttgttgtcaaaattaagtagctcatgcatatactttgagatgcttttcacatgcctgcataaagtaattgttggaccatcgttaatgtctgccataattgcacttaataacaaagcttgtaacctttcaagttctgagattctacaatcttccaaagaaaataaaaggctaatgggaactattcaaaattcatattcagtagcaagcataattaaacatgaaacattaaaaatagaaatttctgtttggctataagaatgcctagacatttgtaatgatcaaaatctgcaggcatcattttctaagagctagactgtaaacaaacctcagaggtaccaactatgccatcagtagtacataaaacatctgatgcacatttagtcacttgatcgatttctcttgaatgagtgaacgaatgaacaaatgaatataagagattaaaattttagccattaagtagaaagaataagaactaaagagaaggtaaaggaggaaaaagagaaggcaaggaagttgagtaagggaagaaatagctctcgtttaagtattttggggactctgttgaaaaaagaaatgccaacatgtggttttaatctttggagctagaactaataatattgtgcaaaagcacaagatgagagatcaagaagttcaccatgacaccttcgctgcttcctggtcttaaacctcagctgaggctggaagaggaccatggtggcttattggagatgtgaccccagggagcccctctgaaggatggaaggggactgggcaagacccaacacacacagaacacagtagccactggccaggcaggaagcaaggatctcagaaaagacttttaggtgaatgtggcaggaaagcgtgcttgctggggcaaaggcagattcattctttctcttcccaggtgacccagcgcctcttggtttctaactggggagggggtaggtgtcaagagatgagtcccaaagttctggaatggtgggtcttgtgactgaggtctagacccctctccagcatgagtgctgtctcctgcatcatatggagcctgggcattctgagctcattcaaagggacaccatgggaaccacttgttctcaatgcaattatttttgtgatgtttacag

SEQ ID NO: 3 - DNA, 75 bp, Homo sapiens
  source 1..75: mol_type = unassigned DNA
tatcagcccccatctaccaacaagaacacgaagtctcagagaaggaaaggaagtacatttgaagaacgcaagtag

SEQ ID NO: 4 - DNA, 15250 bp, Homo sapiens
  source 1..15250: mol_type = unassigned DNA
aggacaggaggattaaacagacagaggcaaggatgatgagagaggagcagacagcaagaatgaaaagcagaaaatacaatagaggaaatgaagaaaagtaggcctgctggagctagatgatgatgtgatggaaatagaagtaaccttttagagaatctcgctaagaaacatggagaaaacggaaaagaaaaatgtaatgccctagaaagcgcaaagaaagacagtggcaaaaatgaaaaaaaaaaataaaaattataaaagaggcaaaaaaagacacactattctctgcctctaaaacacaattaaataaaagaatttaaataaaaattaaggcttctatatgcatttttaaattttgtatgaatctgttatggaagaattgcctatgtcaatatatgttcagagttaaatattagccccaaatgctcagcaagactgaattgtgtcatagaagttcccagattcccttttcccgcaatgtcattggaggctgcatttcttagtcaagtccagggtttaggccaaagggcatccggtattgcctaaaaccctgtgaggtctgtgaggtaacttttgagaagaggtcactgcactcttcatcttttttgcactttggaatcagatataaaagatgtataagtttgctagggctgccataacaaagtatcataggctaggtagtttaaaccacagaaattgattttttcatagttctgggagttgaaagtccaaaatcaaagtatcagcccttgcaagggccttagagaaggctctgtcatgggctcctcccctcggcttgtaggtggcctccttcttctccccctgtgtcttcacttcatcttccctccatacatatctctgtgtctaaacatcctctgtgtgaaacaacaccagccaggttggatttgggcccaccccactgacctcattttaacttaattatctctgtaaagactctgtctccaaatacagtcatattttgacgtactgggagttagggcttcaacacatgaatttggacacaattcagccagtgacagaagacttctgatctctgatgataaccactgcattttgattacagctcctagaaaacactcccctccaccaccccaccacagatctatttttatatctgaaaccctgagtttctgctccatgagaaccccaggaacatactatgttagatctggaagaagcctcagaaatccccttattttgaagactaggacactgagatccagaagtgggtaaagatgtgcttgggttctaagctgctcttcttttggccaggagacaacagcacataatcaaagtgggtcaactaagaaagaattccagaaggaaaagagagggcagaaatgaagggagagaatgagagcaaaagtgctggatttccctgagggtgaagaaaagttaaatagaatcacagaattcagattttagagatcttctccttcagatcccttggtttaatcagtaggattggggtcttcatagataataaagcaaaaactctcgccatcctccaagttgtgaattagaagagctgagaaagggtacaagacggaagttctctaccaaacaaatggtgacattttggggtaagaatatgactaacccagaagtgaagcatttcatccaagtagtctattttgaagatgtcatggtataaaggaacctcctttctgcctggtcctccatgcctctgccatgctttttactccaggatcaccctttctagtggttcactgaaaacccaggattacttaaatatgatggacatgttcacggctcaatccaggaggaaaaggtcgaactgaaagcatgccaaagccccacatgggagccaagccactgctgctgtggttgcaaagtggatcctggcttatcagagcagagagaagccaggctcgtgccttagcccaagtggccagtcaccttattcaggagatactaagttctccagctaagacatccatgctttgggaccagctgcagacagaagccaattcctactacaaccatcaccttagagtagcatatagacacagatggctcttcaaaggaccacagttccatggaataactaagaattcatgtcctgtggaaaggtttgaataaactataattatacccaatcataaatttcattcaagaagaactaaagcaaaggcaaagacagagagaagaaggaaggaaggagggagggagggagggaaggaaggaaggaaggaaggaaggaaggaaggaaggaaggaaggaaggaaggaaagggaaggaagaacaaaaagactttctagttaaagaatgcttaactagcaaactatgtactataagacagttcttttcggaatgagttttatcaactctaaagcaattatcttgaatgcctacatgtgattactgaataatatgaaccaagaaaacagaaagaatctatattatctttccatttccttctttccagtatcaatacccaagcctctagtgatacatggcatataatgttggatggatggatggatggatggatggatggatggatggatggatggatgaatggatggttggatggacaaatgagtaacataggctgatgaatagtggtagaaagacacaccataaaaacaagtggcacttctgagatgaaatgattcctattctcctacacaagacagtgaggcaagtacagagtaaaaaaggaaaggcataggagctatgcttatacaagtattgtatgtttggaatttccttcgctggccaaattgaaattgttcaaggacctattgctacaggtggcaactggctaagaatttcatagtgaatattatacacctattactccccttaatgtttctttgaagtaagcagaatattaataatcatttaaaattccagtgtttcaacttcaattgtttcctagggcaaattgataattgtgtgtaaaactaattggaatatgtatggaataatcatcctgaaataaaattggtgaaaagtatttgttattgggcatctacaatgtgcaaacctctgtactaggcatgaacaagagttataagcattggagaggctaaaatatagtccttaaggctgggcacagtggctcatgcctgtaatcctagcactttgggaggccaaggcgggcagattgcctgagctcaggagttcaagaccagcctgggcaacatagcgaaaccccatctctactaaaaatacaaaaaaattacctgggcatggtggcacgcacctgtaatcccagctactcaggaggctgaggcatgagaattccttgaacctgggaggcagaggttgcagcgagccgagatcctgccgctgcatcccagcttgggtgacagagtgagactctgtctcaaaaaaaaattaaataataaataaatagtaaaatacagtcattaagagtacaaaatgtagattcagactacctgggttcaaatcttggctcttacttgcattgtggctttgggcagatcatgtaacttatgtgtgcctcagtttcctcatctgttaaataggggcaacaactgaatctaccttattcagttgttgtgagggtttattgagattgtgtgtgtgtatgtgtgtgagtgtagtgtgtgcatgtgtgtgtctgtgcaaggagtgggaggtgtatattcagagacacatattacagcacttaaaatggtatctagcacttagtaagcattattcaagttttagttaacattattttacttacctctgaaaattggagctatgtgaaaaagaagttggtctcctgaagtagaagccagtcttgtgtcaccaaaaacttcaagcccaagcttgccaacgcttttccatgatgtggtagtagagtttcaagcatgtggtaggataagagaactcaatgacctaagaaccattccaacccagagaacccctggttctatgaataattccaacttaaataggtagcttggctctcccaagtgagagccattgcttctgtttccgggtcatataatgaactttcagaaaaccaccatttttctcaaccagttaaaattaagtgtaatacgtgctttcatttcatggtgcctggggaaaatttaattgtagtatgaactccagttattggtagtcttaagtaaaattgccaaaataaatagaaatgcaggatatttctgggctcacacagcttccgggacactttagtttcttgggctgccaatccagtgcctttcacaagcatttgatcttttttcaaacatctcttgaaaacaaacaaaacctcacacagcttctaatgtgtgcactgttcgaatgtaagggtggaaaaggaggcaaagaaatgagctcccaaagagcaattccccttctctcgcctccatcccttgacgacctccctcccactaaagggaaacattgttttcttaggtaataaattctgcaatttctcaagtccattaacatccactgggcaagatgagatctattctttttatttgcccataggaaaagaatagtgcttttttgcaatattcactagataacacagagttgacttttaatccaagggcaacattgatagtctctagttaaaggggaagccttcaggagcaatgaaaagattaatagttttagatgaagcagaatccaaatccctttttatgagttttgaaatatccagtttgtatgctcacctcaatacttaaagcccagttactgattcctttggcctaagcaagacaggtcaatttttaaagagggagtagctgaggttagcaaaaattctccaggtccacaaaacttccagacctgcaaggtgaaaatcagcttttctgtcatccctaaaggcctaactggaatcagaacttttccctgatgcccacatatttggaggtccttttttaatgggactccttaatgcctttagtgccatcccattttcatccagtgtccaaaagaaatgatttaaaaatataaacgtatgtttaaattccagaagagagaaatggagattgagaacaatagggaaatgatgagagctatgggaaaagaggtttatgagtccatgtctgattcttccagagagcccctaagaaagttcttatcataccaggaactcaattataactttcattgcctattgttagatgagtaacaggagctagaaaacattttggaaattcccatctttatttttttaactaatatgattatagttttaagaaccattggtcaagaagctaactttttaaaaagtggaagtatgatggttagaaataagaatgctaaaggtgcatcaagctgattttaattctaaatgtccttggcagcaatttagaatctgtaataaactacaccaaacagttttgaggggaaggggattagtttctccccttccttcgtgtgtgtgtgtgcgcgtgtgtgtgtgtgcacctttgtgttctagcattgttgcacccattacagagctggggggaactattttccaaaattataggtgagaacagtttcttggattgtctttcagtgaaggtaaattcctctgtaaaaactaaccatcattcagtaaaaactgcaggattcctttgtcttctcaaaagcctgtttctcatcctaaattaaaaattattcaggaaatagagaggacattattggaggggtggaaataagttggttttctttttattgtatcttttgaggatccagggacttctaccatttcccatctaacatacagagaaggattctctaggtccctgtctatagactgcagtaactttcctatagaaccaatttgcaattttagaaatttctaggtctaattattgacccattacaaccaaaggtcaatgcatccagccaatcttccttctatcatcccctgcccttacttctattagggactgggattacaggcaaaacccatcaaatgcctcttctaccactttcccatttcttaaccattagcctctaacttcctctattcagtttctcatatgctttcatgcccattgggtcagataaaggaacattcatttatttgagtaggcatctgttatgatcactccggaaaaaagatgacaatgggttaccttgtcctcctgggcttctctaactgacatggtcaaaatgcccatatgaagataagatgttaagagcaagatttatgaaaagctgagtatgatggcagctcttgtctcataaaataactcgaaagttcccagtgaaagaccaagaaattttacatcaaacccaaaccggccaaatggtccaagcttccaagctgggatccatggctaaagtttctacaaaattctgggtacaatgtataaacattcacttggggctttctgtctagccagcaccaagaggtcaagtaatcaaggaccaactagccctgccatctgtgaaaatatgtgctattttcacggctttagttcacaattatggcaagacaaaagttccaaataattaggagcaagaccatggcaggttgacggttgagtaaggttctcaatcagccgacaattgtagagttggggatgtgcaatgtttatgtcatggtgtaagtatgtggcatgcttgactagcttgtgaggcactggaagactagaaggaatgaaaaatatgaatgaatcaataaatgcatagtataattactgttattttgtcagtattgttttacctaggtcactattgaatgctctgatttgtctctttataaataataatatgttttcttcttcaaaagaacactaggatgaaggtagaggtgcttttggcacaatgccacaattctgatttttttaaaactgtatgcatgcataaaatgttcttgagccattctctgccttggaatagcactggctggcattctgcatgtttacttttatatgctgaaggcccccatcaacctcaaacagaggcaaatcaatttaacttctcatagtgttattttgttcatcctaaaagttcaagagagccttccaaacttccaaaatttctctcaattcagtgaggaggaaaattcagaacacagcatttgaatgttctgcccagatttgtcacacacacaaggaatgagtgaaagagggcaacaccctttcctcctaaccctgtgaactcatcactattgcattgaaatgacaccaaaaggtaaaaaccctaggcctcacatctcccaagaacactgcaataggagttactgcatacaccagtttaagtaactctagcataaattgtatgtcagatgaaacaatggcattttggaggcttaagagaaaaagaataatcaaatccagtttttaggtactaatgtgctgaatctttagcacatagcagcaaaattgctagaatctggtgtttcactttttaaaataccacatttgaacctttcagcaattccaaaatcaactccctctgcgaaagataataagcttaaacattttttaaatttaaaaatgtaacacaaacaaacagctaagcaaacaagctgcccataaaatcaacagtctggggagccctgatcctgaagtattttacaacatccttcatgactattaaaggcaacataaacacctcttgtcagcaagggaaactacccttggcatttttttttctttgttccccaggcttttaaaccattttgatagagattttttacatcacaggcagaaatatttgaaatagagtcaggtggtagtctttaaaagagtaagaaagttgctaagtcaagataatcttggaataaagtcctctgattcctggggattcctagggatgccccagtcactagaaaacagagctgtaagtccactctcccagcactcaacggagctccggaaaccaaggagctagctactgtttccccacattcagccagagaaagggcagcactctagcatgcaaactgctttgacaatagtaacaattaaaaagtaaattaaaaagaatcataatagctgatattgattaggtacttgccctgtggcaagagctatagggaatcacctcatttaatcttcacatgaagcttgcagagtgagtaccacaattatcactattgtatagacaggaaaactcaggctgagtatggctaagtgtcttgccaacgtcttgggctaacaagcggtcaagcagaatccaaacccgagatagatagaccacagtgtgctaatcaagcactgcactctctcctgcatttcttagttgatatttaccatatacaatctgtcacttgtatgagatggcagggggttctgtgctatttgtccttgtagagaataccacaggaagaaagtaagcagccatgcaatatttgctgttgacctgaactccattccatcattcctgcaggaaattcgcatccattaaatgagcatttcctggtttgccactttgctcaaacactttgcttggatctggagaggatatagaagtgaaggaaatatgctacctgctctcaaggaacttatgttttagtggagagacaaacatgcagaatttactctacagaacatcaatgcttgagcaaatgtagacccagagagggctcttacagcacacaagccagaacagactgatggtgctaacaattaggttcaaggtttttctaaacagtagactctcctgcatacaactataccgcatgccaggtaaatgactgagggttattacatccaattataacaccactgtgatgtaggtgctcttaccccacactttcattttacagaagaggaaattgaggacagcacaatgtagtgattatcaaaggtcacacgactactgtgtgggagagctaggatttaaaccagatgcataagatgaggtcctccaagaaacagaagatgagaaggtgttaaatgagcaggggttttattagggggaattaatgtgtgaacagaaataggggaggataggcaaagccatcagattgcaaggcaagcctaaccccaagggaaggagagagagagagtagattggttggaaacatttttggtgggtctatggtctaaggaaagttcagcaaagtcatcatggagtttttgagccaaagttgggcaatacagttgcccaacaaatttctgtgtttctcagaaataggtctgcctcaatgtccccaccatacttggtcactggctcttgggaggggcctgccctgttccaatccactagagccaaagaagagccgttgtactggcagggggtgggggaattcctacaaccacataaaaagtggggtgaggtttccagaaaaaaacgtgatgctgggctaaccaaaactgtgtccagtaagtacatatccctcactctgttaaagaagcagccacataaacaaggagtacacgtttctcaaaatgtgcaccttgttctttggttttgaagtcacatcccaaagtgctgagtagatcgcatgaccctcgctttgcctggctgccagagaggaaaggctgatccaactctcctggaatttgaacttgtgattccctgaagtaaagagatatcaaagttgatactgagacatctaaatcatcctccaccatttcacatgtccccaggccaagccagcaaaattgctatagcacatccctttcaacaggtaaagggctgatatctgagccctctttccaatcatccactgctcttttcttctcattttgccctttttgggagcaggtcaatgctgagttagtactttatgctgtacaataagctgctgatattccatgctggacagaattttcccagtattttttatagagtgccaggcttttcctagacttcatgtcatacaatacttaacttgtttggagtgggtggagatggaaacatagtctattgaaaacatcactgcttcctccctgaagtttaaagagcctatttttatccttttagattctatctctcaggcaaaatctcataaagataagtggggaggaaaaaaagggggttataatacctagggagtttgcttttgctaattgaatactgtgctcctagacttctataaataccattacaaatgggtcccagcttgtggtaatactcaccctcctcattgagtcttctgtcccatggcacagcctttccctccaaactagcatctacccccatctggaagcatgggcagctcatgatattatcaactattgctattggaaagtgatttggacttgaaagcactagatattttttacctcttggggaggcagtttagcagagtggttaactggtgagctccagaatcagaaggaataggtccaaattccaaccactattacatctccatcataagaaattaggcaagttgtttatcctaagtttcagattccttaaagataaaacagtcaagacagtagtacttatccctgagagaagtataggaaacaagaaaatatatgcaatttacatacatactacaatccccagcacatgacaaatgttcaagtaatgggaactgttattattttagccctttgtctatcagtttgttcctctgtgacctcaagcacattactaaatgttagcgagcttcagcttgtacgtgggactgacaggaataacaccgcatcacctcatgtggtgattgtaaggattcagtgatattattttgtaaactgtaaagcctttgcaaatgttaagcaagattattattattgccgttgttattagtcctcagtgatctttttttttttttttttttttttttttttttttttttttggagacagagttttactctgtcgccaaggctggaatgcagtggcacaatctcagctcactgcaacctccgcctcctgggttcaagcaattttcctgcctcagcctcctgagtagctgaaactacaggcacacgccaccacaccaggctaattttttgtatttttagtagagacggggtttcaccatgttggccaggctggtctccagctcctgacctcaagtgatctgcccacctcggcctcccaaagtgctgggattacaggtgtgagccaccacacctggcacagtaatcttaattgaaaagtctgtggatagctttccaaaggaaagcttggagcttggataagaaccaagagataatgggagaaggtgaatggcctcttcagggccttttctagcaccctaaatatgcgtgtctgtccataatgggtaatcatatatatcacaaatcaaaccctccacaaacttatttcctaatgtgtttgttaacctttccttctaaagggtaaacttctttaaccaaccccagtgagctggaggatcaatgttttcttaatagtcttaccttcgttggtgtcaataggaaacagtatttactcactactgttttccttttaaaaatctgtctagttgcatactagaaacagtttcagctggtttgtttgtattggacaagctgctgaagtgaaaagtttttgcttgactgaatgtgagacagtttcataactcttcaagaagtgcaccaaaggtgggtgccagctctgatgacggctgcttctaacatgcctccacttgccgcccattgtcaagggtggctggcgtaattaagttaagacaatgagcaaagcaacagatgcaactgagacctagtccctgagtgcttttgttttgtcactgtcattgtctgcaacaaagaagtcacatgtgacagcctgggaagagagccaaatgcaaaccagacgatatcccagctggtttgaatggcctccaccgtgcacgtgtgtgcatgggaatcatgctacttggtacagcatctgcttcactcaagtgagtttcagcccatggctttgctgtgatgctgagacagacccagaagaaacagaccagggaatccctccgctcagactttacactttataccttgtgctttgagagaaaagaaaaagaatctctctattggagacaaaaaataggatgtatgtggttggtcaatctaacctcaattctttttgctatagccccccgctaatttaaagagtgaagcatagatggtatcttaatgttttcttgtagaaatttgggattaatttggcttgagaggaagaatggagattaaacgctttatgaggctttcttttaatttgttcccatttcattcctgaatattttcttagtttgggcattgcagatgtttaaagaacttcttattttgagctggtatgcctcttaaacagaaaaacaaaaggtaaaattcaaattagtgtgtttctccgcctgttaattaatttggttagtagttaggcagagagatggcatccttaataatatctattttgcgggtttgatcagctacagaccatcaacagtgttgattgagaattgaacaaaaacatttcaaggagtttgggaacattagggatgctattctgtggccccatgtgtccttctctcatttttctagagaactcctataagaaagcagaacacggccaggcatgatggctcatgcctgtaatcccagcacttcaggaggctgaggcaggcagatcacctgaggtcaggagttcaagaccagcctggccaacatggtgaaaccctatctctattaaaaatacaaaaaattagctgggcatgatggcgcgtgcctgtaatcccagctacttgggaggctgaggcaggagaatcacttgaactgggaggcaaaggttgcagtgagcctagatcacaccactgcactccagcctgggtgacagagtgagactccaactcaaaaaaaagaaagaaagaaagaaagaaagcagaacccaatggaagattaagaacacacatttagcttacgcctgtaataccagcactttgggaggccaaggcgggtggatcacaaggtcagaagttcgagaccaacctggccaatatggtgaaaccccatctctactaaaaagtacaaaaattagccatgcatggtggcaggcgtctgtaatcccagctactacagaggctgaggcaggagaatcacttgaacccgggaggcagaggttgcagtgagctgagaacgcgccactgcactccagcctgggtgacagagcgagactccatctcaaaaaaaaaaaacaacaaaaaaaaacaaaacacaagtttactgggaacttagcagtagatgctttgcaccacaacaaatgtatcttaagtggtcttttgtgatatttgagggaaagtgccagaatttaaaacaaatggcatttcaagttattctatacaaatgcccagtttctttctaccatctttttttcctttttgcagtggtcactgagctattttagtgaatgtttttacacaatgatgccatcttccttctactcagtcagtacaagatgttgaccatcgactcataaaacactagctacctttcatgaaggacttggtgataactctcatgttccaagtagaaccggaaaacatgtgtaagaaaacctgccgatccctatgggccttggccaataggtattattcccaaggggtggcagtttatctttttccccagccttcatattaaaacctctcaccttctccaggtctcaggtctgtgtaatctcaaatgtgctttagctcctcacaatattgtaactgtgtgggtgttcattaccttagccagaagacagtttacagattccaggtctcatggagagaacttttgtttttggttatgaacctcactgtataccaataattatccattacatccttctgtagagggctctctggctagagataaaaccaaaaaaagaagtacctcaggtttatgcatataaatgccagttcctccttgattttatttcaaaactcctgtctacatactttgcaatttaaatacattcaaggataaagtaataactgtaggaaaagtattataatataatgacttagttctgcacatcacaagggggtccctcatactcattcattcatttcactcattttacagatatttattgagcacctgcaataacctgcacactgctctagacactgggactataacagtaaacagacagatacatctctggtctcacagggcttctattctaagcaaaactcaatatccaggccgggtgcagtggctcatgcctggaatgccagcactttgggagaccaaggccaggcagatcacctgagcccactagttgaagaccagcctgggcaatatagcaaaaccccgtctctacaaaaaaaaaaaaaaaaaaaaaaaaaaaattgtcaaggcatggtggcatgcgcctgtggtcccagctacttaggaggctgaggcaggaggattgtgtaagcctgggaggcagaggttgcagtgacctgagatggcaccaccacactccagcctgggcaacagagtgagaccctgtccaaaaaaaaaaaaccctcactatccttaagataacatcattgcttgttgatgagtgaatgttaacaccaaattaggaacccaggacttttagtcttggcatggttactttccaataaagatgacaatactaagaagagaaaaatgatttaataatgataatagtggctaatacttatgtagtgcttaccatgtgccaggtctattgtaagtacttttatatatattaattatttaatctttgatcctataaggtagatattattgttaccctagtttatagatgaagaaacggaaacacaagagattgccactcatacaagtttacacagccagaaaatagaaaagctacgagttgagctcagcccagtatgtctatgattttacagactcaaaattaattataagatttcctaatcttcgatttctgaaactctgccttgctctagaggaaaacaagaaaaacaatgaaaaataaatgtctcttttttacaaaaattaaaacagaacaaactgcaataaaacaacagaggatgaatccagaatgtgattgattttttttcttactaggaaaggatctagaggccagaaggctggatttttcaggatctcctttcaatcaatgaatctgtgatagaagcagatgaatcaaatctcatctttgtgtgattataaagctgtctgtggtattcacgccaccaggggtacatagaagatgcctgagtgaggtttggcaaaagtactaagggcctgtccacctatacatgcccttctcaggaaaaccaaggttcaagctctctattagctcaactggtaaggcgtaagacatggaaggttgaggcccaatgttagaaatagatggatacataaaacttcatcaagttaatgtcactttttctcctttatttatag

SEQ ID NO: 5 - DNA, 60 bp, Homo sapiens
  source 1..60: mol_type = unassigned DNA
gaagtacatttgaagaacgcaagtagagggagtgcaggaaacaagaactacaggatgtag

SEQ ID NO: 6 - DNA, 375 bp, Homo sapiens
  source 1..375: mol_type = unassigned DNA
gtaagcccacctgggtgggatccagccatcctcaagtggtctctctcttgtgcatgtgggtgggccaagcagaaatcctgccccatagtctcctggcttacaagtcagaaaagctcctttgcaccaaagggatggattacatccccatctctttggtcactctgcattgcaaatttcccctcccaccgctatggacgatgtgatgattggaagatgttacaaaacagtggctaaacaaacatgggctttggtgtcagacaaaagtgaagtcctggctttctcacacaccagcttagagagaaaagacttttaggtgaatgtggcaggaaagcgtgcttgctggggcaaaggcagattcattctttctcttcccag

SEQ ID NO: 7 - DNA, 300 bp, Homo sapiens
  source 1..300: mol_type = unassigned DNA
gtaagcccacctgggtgggatccagccatcctcaagtggtctctctcttgtgcatgtgggtgggccaagcagaaatcctgccccatagtctcctggcttacaagtcagaaaagctcctttgcaccaaagggatggattacatccccatctctttgctaaacaaacatgggctttggtgtcagacaaaagtgaagtcctggctttctcacacaccagcttagagagaaaagacttttaggtgaatgtggcaggaaagcgtgcttgctggggcaaaggcagattcattctttctcttcccag

SEQ ID NO: 8 - DNA, 21 bp, Homo sapiens
  source 1..21: mol_type = unassigned DNA
ctttttctcctttatttatag

SEQ ID NO: 9 - DNA, 933 bp, synthetic construct
  misc_feature 1..933: note = Description of Artificial Sequence: Synthetic polynucleotide
  source 1..933: mol_type = other DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggtaagcccacctgggtgggatccagccatcctcaagtggtctctctcttgtgcatgtgggtgggccaagcagaaatcctgccccatagtctcctggcttacaagtcagaaaagctcctttgcaccaaagggatggattacatccccatctctttggtcactctgcattgcaaatttcccctcccaccgctatggacgatgtgatgattggaagatgttacaaaacagtggctaaacaaacatgggctttggtgtcagacaaaagtgaagtcctggctttctcacacaccagcttagagagaaaagacttttaggtgaatgtggcaggaaagcgtgcttgctggggcaaaggcagattcattctttctcttcccagtatcagcccccatctaccaacaagaacacgaagtctcagagaaggaaaggaagtacatttgaagaacgcaagtagctttttctcctttatttataggaagtacatttgaagaacgcaagtagagggagtgcaggaaacaagaactacaggatgtag

SEQ ID NO: 10 - DNA, 858 bp, synthetic construct
  misc_feature 1..858: note = Description of Artificial Sequence: Synthetic polynucleotide
  source 1..858: mol_type = other DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggtaagcccacctgggtgggatccagccatcctcaagtggtctctctcttgtgcatgtgggtgggccaagcagaaatcctgccccatagtctcctggcttacaagtcagaaaagctcctttgcaccaaagggatggattacatccccatctctttgctaaacaaacatgggctttggtgtcagacaaaagtgaagtcctggctttctcacacaccagcttagagagaaaagacttttaggtgaatgtggcaggaaagcgtgcttgctggggcaaaggcagattcattctttctcttcccagtatcagcccccatctaccaacaagaacacgaagtctcagagaaggaaaggaagtacatttgaagaacgcaagtagctttttctcctttatttataggaagtacatttgaagaacgcaagtagagggagtgcaggaaacaagaactacaggatgtag

SEQ ID NO: 11 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 12 - protein, 723 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 13 - DNA, 3679 bp, synthetic construct
  misc_feature 1..3679: note = Description of Artificial Sequence: Synthetic polynucleotide
  source 1..3679: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatcctgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 14 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 15 - DNA, 462 bp, Homo sapiens
  source 1..462: mol_type = unassigned DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 16 - protein, 158 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 17 - DNA, 477 bp, Homo sapiens
  source 1..477: mol_type = unassigned DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacgcaagtag

SEQ ID NO: 18 - protein, 137 aa, Homo sapiens
MITPTVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 19 - DNA, 414 bp, Homo sapiens
  source 1..414: mol_type = unassigned DNA
atgattacacctacagtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 20 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 21 - DNA, 588 bp, Homo sapiens
  source 1..588: mol_type = unassigned DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggttggccaaagacacatccaggaggggaacagaaggaggggacagaagcaagtctgcagatcagaggaaagaagaaagagcagaggagggagattggaagtagaaatgctgaatgcagaggcaaaaaaggaaaatga

SEQ ID NO: 22 - DNA, 482 bp, Homo sapiens
  source 1..482: mol_type = unassigned DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacag

SEQ ID NO: 23 - DNA, 1675 bp, Homo sapiens
  source 1..1675: mol_type = unassigned DNA
cctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 24 - DNA, 3756 bp, synthetic construct
  misc_feature 1..3756: note = Description of Artificial Sequence: Synthetic polynucleotide
  source 1..3756: mol_type = other DNA
cgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 25 - DNA, 4956 bp, Homo sapiens
  source 1..4956: mol_type = unassigned DNA
gtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatccagtatattaataaaatccctttttgtattcaatgagggaaacacataattttcatcaattagcagcttattggaatatctgcatgatggtttaacacttttaagtgttgactaaagattaattttacagaaaatagaaaaagaaatatgtttctgtctggaggaatgatttattgttgacccctaaattgaaatattttactagtggcttaatggaaagatgatgaaagatgatgaaattaatgtagaagcttaactagaaaatcaggtgacctgatatctacatctgtatccttcattggccacccagcattcattaatgaatcagatgatggaatagatcaagtttcctaggaacacagtgaatattaaaagaaaacaaagggagcctagcacctagaagacctagtttatatttcaaagtatatttggatgtaacccaattttaaacatttcctcacttgtctctcttaaagccttgccaacagcaaggacagagaaccaaaaatagtgtatatatgaataaatgcttattacagaatctgctgactggcacatgctttgtgtgtaatgggttctcataaacacttgttgaatgaacacacataagtgaaagagcatggctaggcttcatcccttggtcaaatatggggtgctaaagaaaagcaggggaaatacattgggacactaacaaaaaaaaacagttaatttaggtaaaagataaaatacaccacagaatgaagaaaagagatgacccagactgctctttaaccttcatgtcctagagaggtttttgatatgaattgcattcagaattgtggaaaggagcccatcttttctcttcattttgattttattaactccaatgggggaattttattcgtgttttggccatatctacttttgatttctacattattctctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaaga

SEQ ID NO: 26 - DNA, 7113 bp, synthetic construct
  misc_feature 1..7113: note = Description of Artificial Sequence: Synthetic polynucleotide
  source 1..7113: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatccagtatattaataaaatccctttttgtattcaatgagggaaacacataattttcatcaattagcagcttattggaatatctgcatgatggtttaacacttttaagtgttgactaaagattaattttacagaaaatagaaaaagaaatatgtttctgtctggaggaatgatttattgttgacccctaaattgaaatattttactagtggcttaatggaaagatgatgaaagatgatgaaattaatgtagaagcttaactagaaaatcaggtgacctgatatctacatctgtatccttcattggccacccagcattcattaatgaatcagatgatggaatagatcaagtttcctaggaacacagtgaatattaaaagaaaacaaagggagcctagcacctagaagacctagtttatatttcaaagtatatttggatgtaacccaattttaaacatttcctcacttgtctctcttaaagccttgccaacagcaaggacagagaaccaaaaatagtgtatatatgaataaatgcttattacagaatctgctgactggcacatgctttgtgtgtaatgggttctcataaacacttgttgaatgaacacacataagtgaaagagcatggctaggcttcatcccttggtcaaatatggggtgctaaagaaaagcaggggaaatacattgggacactaacaaaaaaaaacagttaatttaggtaaaagataaaatacaccacagaatgaagaaaagagatgacccagactgctctttaaccttcatgtcctagagaggtttttgatatgaattgcattcagaattgtggaaaggagcccatcttttctcttcattttgattttattaactccaatgggggaattttattcgtgttttggccatatctacttttgatttctacattattctctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 27 - DNA, 6190 bp, synthetic construct
  misc_feature 1..6190: note = Description of Artificial Sequence: Synthetic polynucleotide
  source 1..6190: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 28 - DNA, 5190 bp, synthetic construct
  misc_feature 1..5190: note = Description of Artificial Sequence: Synthetic polynucleotide
  source 1..5190: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatcctgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 29 - DNA, 4241 bp, synthetic construct
  misc_feature 1..4241: note = Description of Artificial Sequence: Synthetic polynucleotide
  source 1..4241: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatccttatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 30 - DNA, 5602 bp, synthetic construct
  misc_feature 1..5602: note = Description of Artificial Sequence: Synthetic polynucleotide
  source 1..5602: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatccagtatattaataaaatccctttttgtattcaatgagggaaacacataattttcatcaattagcagcttattggaatatctgcatgatggtttaacacttttaagtgttgactaaagattaattttacagaaaatagaaaaagaaatatgtttctgtctggaggaatgatttattgttgacccctaaattgaaatattttactagtggcttaatggaaagatgatgaaagatgatgaaattaatgtagaagcttaactagaaaatcaggtgacctgatatctacatctgtatccttcattggccacccagcattcattaatgaatcagatgatggaatagatcaagtttcctaggaacacagtgaatattaaaagaaaacaaagggagcctagcacctagaagacctagtttatatttcaaagtatatttggatgtaacccaattttaaacatttcctcacttgtctctcttaaagccttgccaacagcaaggacagagaaccaaaaatagtgtatatatgaataaatgcttattacagaatctgctgactggcacatgctttgtgtgtaatgggttctcataaacacttgttgaatgaacacacataagtgaaagagcatggctaggcttcatcccttggtcaaatatggggtgctaaagaaaagcaggggaaatacattgggacactaacaaaaaaaaacagttaatttaggtaaaagataaaatacaccacagaatgaagaaaagagatgacccagactgctctttaaccttcatgtcctagagaggtttttgatatgaattgcattcagaattgtggaaaggagcccatcttttctcttcattttgattttattaactccaatgggggaattttattcgtgttttggccatatctacttttgatttctacattattctctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 31 - DNA, 4680 bp, synthetic construct
  misc_feature 1..4680: note = Description of Artificial Sequence: Synthetic polynucleotide
  source 1..4680: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatctcttcctttctacctgtatttgtcctaataaattgttgacttattaatytcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 32 - DNA, 2730 bp, synthetic construct
  misc_feature 1..2730: note = Description of Artificial Sequence: Synthetic polynucleotide
  source 1..2730: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatccttatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 33 - DNA, 2187 bp, synthetic construct
  misc_feature 1..2187: note = Description of Artificial Sequence: Synthetic polynucleotide
  source 1..2187: mol_type = other DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 34 - DNA, 40 bp, synthetic construct
  misc_feature 1..40: note = Description of Artificial Sequence: Synthetic primer
  source 1..40: mol_type = other DNA
agctggcaattccggttcgcttgctgcgtcagaccccgta

SEQ ID NO: 35 - DNA, 40 bp, synthetic construct
  misc_feature 1..40: note = Description of Artificial Sequence: Synthetic primer
  source 1..40: mol_type = other DNA
tacggggtctgacgcagcaagcgaaccggaattgccagct

SEQ ID NO: 36 - DNA, 40 bp, synthetic construct
  misc_feature 1..40: note = Description of Artificial Sequence: Synthetic primer
  source 1..40: mol_type = other DNA
ctaatccataacatggctctagacttaaggcagcggcaga

SEQ ID NO: 37 - DNA, 40 bp, synthetic construct
  misc_feature 1..40: note = Description of Artificial Sequence: Synthetic primer
  source 1..40: mol_type = other DNA
tctgccgctgccttaagtctagagccatgttatggattag

SEQ ID NO: 38 - DNA, 3250 bp, synthetic construct
  misc_feature 1..3250: note = Description of Artificial Sequence: Synthetic polynucleotide
  source 1..3250: mol_type = other DNA
cagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctagacttaaggcagcggcagaagaagatgtaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttatcgatgtcgacctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 39 - DNA, 4108 bp, synthetic construct
  misc_feature 1..4108: note = Description of Artificial Sequence: Synthetic polynucleotide
  source 1..4108: mol_type = other DNA
cagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctagacttaaggcagcggcagaagaagatgtaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttatcgatatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggtaagcccacctgggtgggatccagccatcctcaagtggtctctctcttgtgcatgtgggtgggccaagcagaaatcctgccccatagtctcctggcttacaagtcagaaaagctcctttgcaccaaagggatggattacatccccatctctttgctaaacaaacatgggctttggtgtcagacaaaagtgaagtcctggctttctcacacaccagcttagagagaaaagacttttaggtgaatgtggcaggaaagcgtgcttgctggggcaaaggcagattcattctttctcttcccagtatcagcccccatctaccaacaagaacacgaagtctcagagaaggaaaggaagtacatttgaagaacgcaagtagctttttctcctttatttataggaagtacatttgaagaacgcaagtagagggagtgcaggaaacaagaactacaggatgtaggtcgacctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 40 - DNA, 18 bp, synthetic construct
  misc_feature 1..18: note = Description of Artificial Sequence: Synthetic primer
  source 1..18: mol_type = other DNA
tgatctaaggaggctgga

SEQ ID NO: 41 - DNA, 20 bp, synthetic construct
  misc_feature 1..20: note = Description of Artificial Sequence: Synthetic primer
  source 1..20: mol_type = other DNA
ctacttgcgttcttcaaatg